(19) United States
(12) Patent Application Publication
(10) Pub. No.: US 2008/0255161 A1
(43) Pub. Date: Oct. 16, 2008

(54) 3-HYDROQUINAZOLIN-4-ONE DERIVATIVES FOR USE AS STEAROYL CoA DESATURASE INHIBITORS

(76) Inventors: Dmitry Koltun, Foster City, CA (US); Eric Parkhill, San Francisco, CA (US); Melanie Boze, Round Rock, TX (US); Jeff Zablocki, San Mateo, CA (US); Natalya Vasilevich, Moscow (RU); Elena Mayboroda, Moscow (RU); Andrei Glushkov, Moscow (RU); Andrew Cole, Robbinsville, NJ (US); Jeffrey Chisholm, Mountain View, CA (US)

Correspondence Address: CV THERAPEUTICS, INC., 3172 PORTER DRIVE, PALO ALTO, CA, 94304, US

(21) Appl. No.: 12/100,399

(22) Filed: Apr. 9, 2008

Related U.S. Application Data
(60) Provisional application No. 60/911,225, filed on Apr. 11, 2007.

(51) Int. Cl. A61K 31/519; C07D 239/80; C07D 471/04; A61P 9/10; A61K 31/517

(52) U.S. Cl. 514/264.1; 544/287; 514/266.3; 544/279

(57) ABSTRACT

The present invention discloses 3-hydroquinazolin-4-one derivatives for use as inhibitors of stearoyl-CoA desaturase. The compounds are useful in treating and/or preventing various human diseases, mediated by stearoyl-CoA desaturase (SCD) enzymes, especially diseases related to abnormal lipid levels, cancer, cardiovascular disease, diabetes, obesity, metabolic syndrome and the like.

This application claims priority to U.S. Provisional Patent Application Ser. No. 60/911,225, filed Apr. 11, 2007, the entirety of which is herein incorporated by reference.

### FIELD OF THE INVENTION

The present invention relates generally to the field of inhibitors of stearoyl-CoA desaturase, such as 3-hydroquinazolin-4-one derivatives, and uses for such compounds in treating and/or preventing various human diseases, mediated by stearoyl-CoA desaturase (SCD) enzymes, especially diseases related to elevated lipid levels, cardiovascular disease, cancer, diabetes, obesity, metabolic syndrome and the like.

### BACKGROUND

Stearoyl CoA desaturases (SCD's) are Δ9 fatty acid desaturases. The mammalian enzymes are localized to the endoplasmic reticulum and require molecular O₂ and NADH to desaturate saturated fatty acids at the Δ9 position and generate monounsaturated fatty acids and water in the process. The primary substrates for these enzymes are the acyl-CoA derivatives of stearic (C18) and palmitic acids (C16) with the major reaction being the conversion of stearic acid to oleic acid (C18:1). Depending on the species, 2-4 highly homologous isoforms of SCD exist differing primarily in tissue distribution.

The best characterized SCD isozyme is SCD1 which is primarily found in liver, adipose and skeletal muscle. Deletion, mutation or inhibition of SCD1 in mice and rats results in decreased hepatic triglyceride secretion, decreased hepatic steatosis, resistance to weight gain and improvements in insulin sensitivity and glucose uptake (reviewed in Ntambi et al. (2004) Prog Lipid Res 43, 91-104; (2005), Prostaglandins Leukot. Essent. Fatty Acids 73, 35-41; and (2005) Obes. Rev. 6, 169-174. These studies combined with studies in humans showing correlations between surrogates for SCD activity and metabolic syndrome, diabetes and obesity strongly implicate SCD inhibition as a means to treat obesity, diabetes, hypertryglyceridemia and associated diseases and co-morbidities. Studies done using antisense oligonucleotide inhibitors have also demonstrated a correlation between SCD activity and obesity and the onset of diet-induced hepatic insulin resistance; see Jiang et al. (2005) J. Clin. Invest. 115:1030-1038G. and Gutiérrez-Juárez et al. (2006) J. Clin. Invest. 116:1686-1695.

The present invention presents compounds that are useful in inhibiting SCD activity and thus regulating lipid levels and lipid fatty acid composition. These compounds are useful in the treatment of SCD-mediated diseases such as diseases related to dyslipidemia and disorders of lipid metabolism, including, but not limited to diseases related to elevated lipid levels, cardiovascular disease, diabetes, obesity, metabolic syndrome and the like.

### SUMMARY OF THE INVENTION

It is an object of this invention to provide compounds that act as stearoyl-CoA desaturase inhibitors. Accordingly, in a first aspect, the invention relates to compounds of Formula I:

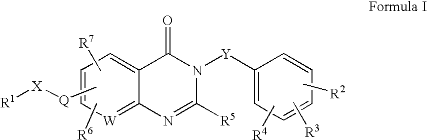

wherein
R¹ is hydrogen, optionally substituted C₁₋₁₅ alkyl, optionally substituted C₂₋₁₅ alkenyl, optionally substituted C₂₋₁₅ alkynyl, optionally substituted mono or bicyclic heterocyclyl, optionally substituted mono or bicyclic aryl, or mono or bicyclic heteroaryl,
R², R³, and R⁴ are independently hydrogen, optionally substituted C₁₋₆ alkyl, optionally substituted C₂₋₆ alkenyl, optionally substituted C₂₋₆ alkynyl, optionally substituted mono or bicyclic heterocyclyl, optionally substituted mono or bicyclic aryl, optionally substituted mono or bicyclic heteroaryl, halo, NO₂, CF₃, CN, OR²⁰, SR²⁰, N(R²⁰)₂, S(O)R²², SO₂R²², SO₂N(R²⁰)₂, S(O)₃R²⁰, P(O)(OR²⁰)₂, SO₂NR²⁰COR²², SO₂NR²⁰CO₂R²², SO₂NR²⁰CON(R²⁰)₂, NR²⁰COR²², NR²⁰CO₂R²², NR²⁰CON(R²⁰)₂, NR²⁰C(NR²⁰)NHR²³COR²⁰, CO₂R²⁰, CON(R²⁰)₂, CONR²⁰SO₂R²², NR²⁰SO₂R²², SO₂NR²⁰CO₂R²², OCONR²SO₂R²², OC(O)R²⁰, C(O)OCH₂OC(O)R²⁰, OCON(R²⁰)₂, or
R² and R³ may join along with the phenyl group to which they are attached to form a heteroaryl bicyclic group or a bicyclic aryl group;
R⁵ is hydrogen or optionally substituted C₁₋₆ alkyl, N(R²⁰)₂, NR²⁰COR²², NR²⁰CO₂R²², or NR²⁰CON(R²⁰)₂;
R⁶ and R⁷ are independently hydrogen or C₁₋₄ alkyl, halo, amino, or CF₃;
Q is —C(O)—NH—, —NH—C(O)—, or —NH—C(O)—C(O)—;
W is —CH— or —N—;
X is a covalent bond or -Lk-Lh-, wherein Lk is a covalent bond or optionally substituted linear or branched C₁₋₄ alkylene and Lh is selected from a covalent bond, —O—, —S—, or —NR″— wherein R″ is hydrogen or C₁₋₆ lower alkyl, provided that Lk and Lh are not both covalent bond;
Y is a covalent bond or -Lk′-Lh′-, wherein Lk′ is a covalent bond or optionally substituted linear or branched C₁₋₆ alkylene and Lh′ is selected from a covalent bond, —O—, —S—, —NR″—, —NR″—C(O)—, or —NR″—S(O)₂— wherein R″ is hydrogen or C₁₋₆ lower alkyl, provided that Lk′ and Lh′ are not both covalent bond; and
R²⁰ and R²² are independently selected from the group consisting of hydrogen, C₁₋₁₅ alkyl, C₂₋₁₅ alkenyl, C₂₋₁₅ alkynyl, heterocyclyl, aryl, and heteroaryl, wherein the alkyl, alkenyl, alkynyl, heterocyclyl, aryl, and heteroaryl moieties are optionally substituted with from 1 to 3 substituents independently selected from halo, alkyl, mono- or dialkylamino, alkyl or aryl or heteroaryl amide, CN, O—C₁₋₆ alkyl, CF₃, aryl, and heteroaryl.

In some embodiments of the invention the R¹ alkyl, alkenyl, alkynyl, aryl, heterocyclyl or heteroaryl moiety is optionally substituted with from 1 to 3 substituents independently selected from the group consisting of alkyl, heterocyclyl, aryl, heteroaryl, halo, NO₂, CF₃, CN, OR⁸, SR⁸, N(R⁸)₂, S(O)R²², SO₂R²², SO₂N(R²⁰)₂, S(O)₃R²⁰, P(O)(OR²⁰)₂, SO₂NR²⁰COR²², SO₂NR²⁰CO₂R²², SO₂NR²⁰CON(R²⁰)₂, NR²⁰COR²², NR²⁰CO₂R²², NR²⁰CON(R²⁰)₂, NR²⁰C(NR²⁰)NHR²³, COR²⁰, CO₂R²⁰, CON(R²⁰)₂, CONR²⁰SO₂R²², NR²⁰SO₂R²², SO₂NR²⁰CO₂R²², OCONR²⁰SO₂R²², OC(O)R²⁰, C(O)OCH₂OC(O)R²⁰, and OCON(R²⁰)₂, and in some cases each optional alkyl, heteroaryl, aryl, and heterocyclyl substituent is further optionally substituted with halo, NO₂, alkyl, CF₃, amino, mono- or di-alkylamino, alkyl or aryl or heteroaryl amide, NR²⁰COR²², NR²⁰SO₂R²², COR²⁰, CO₂R²⁰, CON(R²⁰)₂, NR²⁰CON(R²⁰)₂, OC(O)R²⁰, OC(O)N(R²⁰)₂, S(O)₃R²⁰, P(O)(OR²⁰)₂, SR²⁰, S(O)R²², SO₂R²², SO₂N(R²⁰)₂, CN, or OR²⁰.

In certain embodiments of the invention the R¹ alkyl, alkenyl, alkynyl, aryl, heterocyclyl or heteroaryl moiety is optionally substituted with from 1 to 3 substituents independently selected from the group consisting of alkyl, heterocyclyl, aryl, heteroaryl, halo, NO₂, CF₃, CN, OR⁸, SR⁸, N(R⁸)₂, S(O)R²², SO₂R²², SO₂N(R²⁰)₂, NR²⁰COR²², NR²⁰CO₂R²², NR²⁰CON(R²⁰)₂, COR²⁰, CO₂R²⁰, CON(R²⁰)₂, NR²⁰SO₂R²², and OC(O)R²⁰, and in some cases each optional alkyl, heteroaryl, aryl, and heterocyclyl substituent is further optionally substituted with halo, NO₂, alkyl, CF₃, amino, mono- or di-alkylamino, alkyl or aryl or heteroaryl amide, NR²⁰COR²², NR²⁰SO₂R²², COR²⁰, CO₂R²⁰, CON(R²⁰)₂, SR²⁰, S(O)R²², SO₂R²², SO₂N(R²⁰)₂, CN, or OR²⁰.

In these embodiments R⁸ is hydrogen, C₁₋₄ alkyl, alkenyl, alkynyl, aryl, heterocyclyl, heteroaryl, COR²⁰, or CON(R²⁰)₂ wherein the alkyl, alkenyl, alkynyl, aryl, heterocyclyl and heteroaryl moiety is optionally substituted with from 1 to 3 substituents independently selected from the group consisting of halo, alkyl, NO₂, heterocyclyl, aryl, heteroaryl, CF₃, CN, OR²⁰, SR²⁰, N(R²⁰)₂, OR²⁰, SR²⁰, N(R²⁰)₂, S(O)R²², SO₂R²², SO₂N(R²⁰)₂, COR²⁰, CO₂R²⁰, CON(R²⁰)₂, CONR²⁰SO₂R²², and NR²⁰SO₂R²².

In some embodiments of the invention the R², R³, and R⁴ alkyl, alkenyl, alkynyl, aryl, heterocyclyl, or heteroaryl moieties are optionally substituted with from 1 to 3 substituents independently selected from the group consisting of halo, alkyl, NO₂, heterocyclyl, aryl, heteroaryl, CF₃, CN, OR²⁰, SR²⁰, N(R²⁰)₂, S(O)R²², SO₂R²², SO₂N(R²⁰)₂, S(O)₃R²⁰, P(O)(OR²⁰)₂, SO₂NR²⁰CO²², SO₂NR²⁰CO₂R²², SO₂NR²⁰CON(R²⁰)₂, NR²⁰COR²², NR²⁰CO₂R²², NR²⁰CON(R²⁰)₂, NR²⁰C(NR²⁰)NHR²³, CO²⁰, CO₂R²⁰, CON(R²⁰)₂, CONR²⁰SO₂R²², NR²⁰SO₂R²², SO₂NR²⁰CO₂R²², OCONR²SO₂R²², OC(O)R²⁰, C(O)OCH₂OC(O)R²⁰, and OCON(R²⁰).

In some embodiments of the invention the R², R³, and R⁴ alkyl, alkenyl, alkynyl, aryl, heterocyclyl, or heteroaryl moieties are optionally substituted with from 1 to 3 substituents independently selected from the group consisting of alkyl, heterocyclyl, aryl, heteroaryl, halo, NO₂, CF₃, CN, OR²⁰, SR²⁰, N(R²⁰)₂, S(O)R²², SO₂R²², SO₂N(R²⁰)₂, NR²⁰COR²², NR²⁰CO₂R²², NR²⁰CON(R²⁰)₂, COR²⁰, CO₂R²⁰CON(R²⁰)₂, NR²⁰SO₂R²², and OC(O)R²⁰.

In typical embodiments R²⁰ and R²² are independently selected from the group consisting of hydrogen, C₁₋₁₅ alkyl, C₂₋₁₅ alkenyl, C₂₋₁₅ alkynyl, heterocyclyl, aryl, and heteroaryl, wherein the alkyl, alkenyl, alkynyl, heterocyclyl, aryl, and heteroaryl moieties are optionally substituted with from 1 to 3 substituents independently selected from halo, alkyl, mono- or dialkylamino, alkyl or aryl or heteroaryl amide, CN, O—C₁₋₆ alkyl, CF₃, aryl, and heteroaryl.

In certain embodiments R²⁰ and R²² are independently selected from the group consisting of hydrogen, C₁₋₆ alkyl, C₂₋₆ alkenyl, heterocyclyl, aryl, and heteroaryl, wherein the alkyl, alkenyl, heterocyclyl, aryl, and heteroaryl moieties are optionally substituted with from 1 to 3 substituents independently selected from halo, alkyl, mono- or dialkylamino, CN, O—C₁₋₆ alkyl, or CF₃.

Typical R¹ groups are phenyl optionally substituted at the 3, 4, or 5 position of the phenyl ring with 1 to 3 substituents independently selected from the group consisting of lower alkyl, halogen, CF₃, —OCF₃, and —OCH₃. Further typical R¹ groups are C₁₋₆ alkyl optionally substituted with from 1 to 3 substituents independently selected from lower alkyl, halogen, CF₃, —OCF₃, —O—C₁₋₆ alkyl, or phenyl, wherein the phenyl is optionally substituted with 1 to 3 substituents independently selected from the group consisting of lower alkyl, halogen, CF₃, —OCF₃, and —OCH₃.

In typical embodiments, the R², R³, and R⁴ groups are independently selected from hydrogen; optionally substituted aryl [such as a phenyl optionally substituted (e.g. at the 3, 4, or 5 position of the phenyl ring) with 1 to 3 substituents independently selected from the group consisting of halogen, CF₃, —OCF₃, and —OCH₃]; or C₁₋₆ alkyl (e.g. methyl, ethyl, propyl, butyl, pentyl, hexyl) optionally substituted with 1, 2, or 3 substituents independently selected from the group consisting of hydroxy, halogen, NO₂, C₁₋₆ alkyl, —O—C₁₋₆ alkyl, amino, mono- or di-alkylamino, and CF₃.

In some embodiments, Q is —C(O)—NH—, —NH—C(O)—, or —NH—C(O)—C(O)—. In certain embodiments, Q is —C(O)—NH— or —NH—C(O)—. In other embodiments, Q is —NH—C(O)—C(O)—, in which the —NH—C(O)—C(O)— is oriented such that the terminal C(O) moiety is bound directly to X.

In typical embodiments X is a covalent bond or -Lk-Lh-, wherein Lk is a covalent bond or optionally substituted linear or branched C₁₋₄ alkylene and Lh is selected from a covalent bond, —O—, —S—, or —NR″— wherein R″ is hydrogen or C₁₋₆ lower alkyl, provided that Lk and Lh are not both covalent bond. In some embodiments the X group may be a C₁₋₄ alkylene optionally substituted with one or two substituents selected from hydroxyl, lower alkyl, lower alkoxy, halogen, CF₃, and —OCF₃. Typical X groups are covalent bond, optionally substituted C₁₋₄ alkylene-Lh-, optionally substituted C₂₋₃ alkylene-Lh-, methylene-Lh-, —CH₂CH₂-Lh-, —CH₂CH₂CH₂-Lh-; —CH(CH₃)CH₂-Lh-, —CH₂CH₂CH₂CH₂-Lh-, —C(CH₃)₂CH₂-Lh- or —CH(CH₃)CH₂CH₂-Lh-, wherein Lh is selected from a covalent bond, —O—, —S—, or —NR″—, wherein R″ is hydrogen or C₁₋₆ lower alkyl. Typically, Lh is selected from covalent bond or —O—. In typical embodiments, X is oriented so that Lh is directly connected to the R¹ group; in other embodiments, it is the Lk that is directly connected to the R¹ group. In certain embodiments X is a covalent bond or —O—, —S—, or —NH—.

In typical embodiments Y is a covalent bond or -Lk′-Lh′-, wherein Lk′ is optionally substituted linear or branched C₁₋₆ alkylene and Lh′ is selected from a covalent bond, —O—, —S—, —NR″—, —NR″—C(O)—, or —NR″—S(O)₂—, wherein R″ is hydrogen or C₁₋₆ lower alkyl. In some embodiments the Y group may be a C₁₋₆ alkylene optionally substituted with one or two substituents selected from hydroxyl, lower alkyl, lower alkoxy, halogen, CF₃, and —OCF₃. Typical Y groups are covalent bond, optionally substituted C₁₋₄ alkylene-Lh′-, optionally substituted C₂₋₃ alkylene-Lh′-, methylene-Lh′-, —CH₂CH₂-Lh′-, —CH₂CH₂CH₂-Lh′-; —CH(CH₃)CH₂-Lh′-, —CH₂CH₂CH₂CH₂-Lh′-, —C(CH₃)₂CH₂-Lh′-, —CH(CH₃)CH₂CH₂-Lh′-, or —CH₂CH₂CH₂CH₂CH₂-Lh′-, wherein Lh′ is selected from a covalent bond, —O—, —S—, —NR″—, or —NR″—C(O)—, or —NR″—S(O)₂—, wherein R″ is hydrogen or C₁₋₆ lower alkyl. Typically, Lh′ is selected from covalent bond or —O—. In typical embodiments, Y is oriented so that Lk′ is directly connected to the annular nitrogen to which Y is attached in Formula I. In embodiments in which Lh′ is selected from —NR″—C(O)— or —NR″—S(O)₂—, the Lh′ group is oriented so that the nitrogen of the Lh′ group is bound directly to the Lk′ group. In certain embodiments, Y is linear or branched alkylene optionally substituted with hydroxy, lower alkoxy, amino, cyano, or ═O.

In one aspect of the invention, the R¹XQ moiety is attached to the 6 position of the 3-hydroquinazolin-4-one and the compound has the structure of Formula Ia:

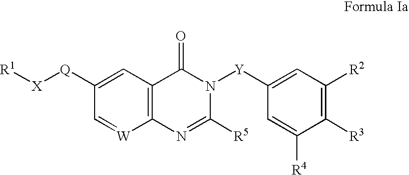

In another aspect of the invention, the R¹XQ moiety is attached to the 7 position of the 3-hydroquinazolin-4-one and the compound has the structure of Formula Ib:

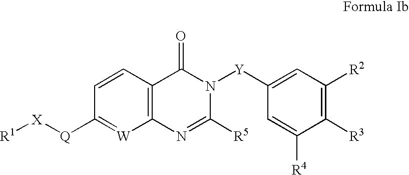

It is another object of the invention to provide pharmaceutical formulations comprising a therapeutically effective amount of an SCD inhibitory compound of Formula I, and at least one pharmaceutically acceptable carrier. The formulation is typically for oral administration, but in some embodiments may be provided for administration via other routes.

In a third object of the invention, methods of using the compounds of Formula I in the treatment of a disease or condition in a mammal that can be treated with an SCD inhibitory compound are provided. The method comprises administering to a mammal in need thereof a therapeutically effective dose of a compound of Formula I. Such diseases include, but are not limited to, cardiovascular diseases (including, but not limited to, coronary artery disease, atherosclerosis, heart disease, hypertension, and peripheral vascular disease), cancer, cerebrovascular diseases (including, but not limited to, stroke, ischemic stroke and transient ischemic attack (TIA), and ischemic retinopathy), dyslipidemia, obesity, diabetes, insulin resistance, decreased glucose tolerance, non-insulin-dependent diabetes mellitus, Type II diabetes, Type I diabetes, and other diabetic complications.

At present, selected compounds for use in the invention include, but are not limited to:
N-{3-[(3,4-dichlorophenyl)methyl]-4-oxo(3-hydroquinazolin-6-yl)}(phenylmethoxy)carboxamide;
N-{3-[(3,4-dichlorophenyl)methyl]-4-oxo(3-hydroquinazolin-6-yl)}-3-phenylpropanamide;
N-{3-[(3,4-dichlorophenyl)methyl]-4-oxo-3-hydroquinazolin-6-yl}hexanamide;
N-{3-[(3,4-dichlorophenyl)methyl]-4-oxo(3-hydroquinazolin-6-yl)}(pentylamino)carboxamide;
N-{3-[(3,4-dichlorophenyl)methyl]-4-oxo(3-hydroquinazolin-6-yl)}[benzylamino]carboxamide;
N-{3-[(3,4-dichlorophenyl)methyl]-2-methyl-4-oxo(3-hydroquinazolin-6-yl)}(phenylmethoxy)carboxamide;
N-{3-[(3,4-dichlorophenyl)methyl]-4-oxo(3-hydroquinazolin-6-yl)}(2-methylpropoxy)carboxamide;
N-{3-[(4-chlorophenyl)methyl]-4-oxo(3-hydroquinazolin-6-yl)}(phenylmethoxy)carboxamide;
N-{3-[(3,4-dichlorophenyl)methyl]-4-oxo(3-hydroquinazolin-6-yl)}(methylethoxy)carboxamide;
N-{3-[(3,4-dichlorophenyl)methyl]-4-oxo(3-hydroquinazolin-6-yl)}prop-2-enyloxycarboxamide;
N-{3-[(3,4-dichlorophenyl)methyl]-4-oxo(3-hydroquinazolin-6-yl)}(4-methylphenoxy)carboxamide;
N-{3-[(3,4-dichlorophenyl)methyl]-4-oxo(3-hydroquinazolin-6-yl)}(4-fluorophenoxy)carboxamide;
N-{3-[(3,4-dichlorophenyl)methyl]-4-oxo(3-hydroquinazolin-6-yl)}methoxycarboxamide;
N-{3-[(4-chlorophenyl)methyl]-4-oxo(3-hydroquinazolin-6-yl)}prop-2-enyloxycarboxamide;
N-{3-[(3,4-dichlorophenyl)methyl]-4-oxo(3-hydroquinazolin-6-yl)}-2-phenoxyacetamide;
N-{3-[2-(4-chlorophenyl)ethyl]-4-oxo(3-hydroquinazolin-6-yl)}(phenylmethoxy)carboxamide;
N-{3-[(3,4-dichlorophenyl)methyl]-4-oxo(3-hydroquinazolin-6-yl)}-2-(4-methoxyphenoxy)acetamide;
N-{3-[(3,4-dichlorophenyl)methyl]-4-oxo(3-hydroquinazolin-6-yl)}-2-(4-fluorophenoxy)acetamide;
2-(acetylamino)-N-{3-[(3,4-dichlorophenyl)methyl]-4-oxo(3-hydroquinazolin-6-yl)}acetamide;
N-{3-[(3,4-dichlorophenyl)methyl]-4-oxo(3-hydroquinazolin-6-yl)}-2-(3,5-dimethylphenoxy)acetamide;
N-{3-[(3,4-dichlorophenyl)methyl]-4-oxo(3-hydroquinazolin-6-yl)}-2-(2,6-dimethylphenoxy)acetamide;
N-{3-[(3,4-dichlorophenyl)methyl]-4-oxo(3-hydroquinazolin-6-yl)}-2-(4-methylphenoxy)acetamide;
N-{3-[(3,4-dichlorophenyl)methyl]-4-oxo(3-hydroquinazolin-6-yl)}(3-pyridylmethoxy)carboxamide;
N-{3-[(4-fluorophenyl)methyl]-4-oxo(3-hydroquinazolin-6-yl)}(phenylmethoxy)carboxamide;
N-{3-[(3,4-dichlorophenyl)methyl]-4-oxo(3-hydroquinazolin-6-yl)}-2-(2-methoxyphenoxy)acetamide;
N-{3-[(3,4-dichlorophenyl)methyl]-4-oxo(3-hydroquinazolin-6-yl)}-2-(2-fluorophenoxy)acetamide;
N-{3-[(3,4-dichlorophenyl)methyl]-4-oxo(3-hydroquinazolin-6-yl)}-2-(3-fluorophenoxy)acetamide;
N-{3-[(3,4-dichlorophenyl)methyl]-4-oxo(3-hydroquinazolin-6-yl)}-2-(2,4-difluorophenoxy)acetamide;
N-{3-[(3,4-dichlorophenyl)methyl]-4-oxo(3-hydroquinazolin-6-yl)}-2-(2-chlorophenoxy)acetamide;
N-{3-[(3,4-dichlorophenyl)methyl]-4-oxo(3-hydroquinazolin-6-yl)}-2-(2,3-dichlorophenoxy)acetamide;
N-{3-[(3,4-dichlorophenyl)methyl]-4-oxo(3-hydroquinazolin-6-yl)}-2-(2-nitrophenoxy)acetamide;
N-{3-[(3,4-dichlorophenyl)methyl]-4-oxo(3-hydroquinazolin-6-yl)}-2-(2-naphthyloxy)acetamide;
N-{3-[(3,4-dichlorophenyl)methyl]-4-oxo(3-hydroquinazolin-6-yl)}-3-phenoxypropanamide;
N-{3-[(3,4-dichlorophenyl)methyl]-4-oxo(3-hydroquinazolin-6-yl)}-2-(2-chlorophenoxy)propanamide;
2-benzo[c]1,2,5-thiadiazol-4-yloxy-N-{3-[(3,4-dichlorophenyl)methyl]-4-oxo(3-hydroquinazolin-6-yl)}acetamide;
N-{3-[(3,4-dichlorophenyl)methyl]-4-oxo(3-hydroquinazolin-6-yl)}-2-(3-pyridyloxy)acetamide;
N-{3-[(4-chlorophenyl)methyl]-4-oxo(3-hydroquinazolin-6-yl)}-2-(4-pyridylthio)acetamide;
N-{3-[(4-bromophenyl)methyl]-4-oxo(3-hydroquinazolin-6-yl)}-2-phenoxyacetamide;
N-{3-[(4-chloro-3-fluorophenyl)methyl]-4-oxo(3-hydroquinazolin-6-yl)}-2-phenoxyacetamide;
N-{3-[(3-chloro-4-fluorophenyl)methyl]-4-oxo(3-hydroquinazolin-6-yl)}-2-phenoxyacetamide;
N-[3-(benzo[b]thiophen-6-ylmethyl)-4-oxo(3-hydroquinazolin-6-yl)]-2-phenoxyacetamide;
N-{3-[(3,4-dichlorophenyl)methyl]-4-oxo(3-hydroquinazolin-6-yl)}-4-oxo-4-phenylbutanamide;
N-{3-[(4-chlorophenyl)methyl]-4-oxo(3-hydroquinazolin-6-yl)}-2-(3-fluorophenoxy)acetamide;
N-{3-[(4-chlorophenyl)methyl]-4-oxo(3-hydroquinazolin-7-yl)}-2-phenoxyacetamide;
N-[3-(benzo[b]thiophen-5-ylmethyl)-4-oxo(3-hydroquinazolin-6-yl)](phenylmethoxy)carboxamide;
N-{3-[(3-chloro-4-fluorophenyl)methyl]-4-oxo(3-hydroquinazolin-6-yl)}(phenylmethoxy)carboxamide;
(N-{3-[(3,4-dichlorophenyl)methyl]-4-oxo-3-hydroquinazolin-6-yl}carbamoyl)methyl acetate;
N-{3-[(4-chlorophenyl)methyl]-4-oxo(3-hydroquinazolin-7-yl)}(phenylmethoxy)carboxamide;
N-{3-[(4-chlorophenyl)methyl]-4-oxo(3-hydroquinazolin-7-yl)}-2-(3-pyridyloxy)acetamide;
N-{3-[(3,4-dichlorophenyl)methyl]-4-oxo(3-hydroquinazolin-7-yl)}-2-(2-methoxyphenoxy)acetamide;
N-{3-[(3,4-dichlorophenyl)methyl]-4-oxo(3-hydroquinazolin-7-yl)}-2-(3-pyridyloxy)acetamide;
N-{3-[(3,4-dichlorophenyl)methyl]-4-oxo(3-hydroquinazolin-7-yl)}(phenylmethoxy)carboxamide;
N-{3-[(3,4-dichlorophenyl)methyl]-4-oxo(3-hydroquinazolin-7-yl)}-2-phenoxyacetamide;
N-{3-[(3,4-dichlorophenyl)methyl]-4-oxo(3-hydroquinazolin-6-yl)}-2-(4-pyridylthio)acetamide;
N-{3-[(3,4-dichlorophenyl)methyl]-4-oxo(3-hydroquinazolin-6-yl)}-2-pyrimidin-2-ylthioacetamide;
N-{3-[(3,4-dichlorophenyl)methyl]-4-oxo(3-hydroquinazolin-6-yl)}-2-(4-chlorophenylthio)acetamide;
N-{3-[(4-chlorophenyl)methyl]-4-oxo(3-hydroquinazolin-6-yl)}[5-(imidazolylmethyl)(2-furyl)]carboxamide;
N-{3-[(3-chlorophenyl)methyl]-4-oxo(3-hydroquinazolin-7-yl)}(phenylmethoxy)carboxamide;
N-{3-[(3-chlorophenyl)methyl]-4-oxo(3-hydroquinazolin-7-yl)}(3-pyridylmethoxy)carboxamide;
N-{3-[(3,4-dichlorophenyl)methyl]-4-oxo(3-hydroquinazolin-6-yl)}-2-(phenylmethylthio)acetamide;
N-{3-[(3,4-dichlorophenyl)methyl]-4-oxo(3-hydroquinazolin-7-yl)}[(2-methoxyphenyl)methoxy]carboxamide;
N-{3-[(3,4-dichlorophenyl)methyl]-4-oxo(3-hydroquinazolin-6-yl)}-2-hydroxyacetamide;
N-{3-[(3,4-dichlorophenyl)methyl]-4-oxo(3-hydroquinazolin-6-yl)}(2-furylmethoxy)carboxamide;
(N-{3-[(3,4-dichlorophenyl)methyl]-4-oxo-3-hydroquinazolin-7-yl}carbamoyl)methyl acetate;
(N-{3-[(3-chlorophenyl)methyl]-4-oxo-3-hydroquinazolin-7-yl}carbamoyl)methyl acetate;
(N-{3-[(4-chlorophenyl)methyl]-4-oxo-3-hydroquinazolin-7-yl}carbamoyl)methyl acetate;
N-{3-[(3,4-dichlorophenyl)methyl]-4-oxo(3-hydroquinazolin-7-yl)}(2-methoxyethoxy)carboxamide;
N-{3-[(3-chlorophenyl)methyl]-4-oxo(3-hydroquinazolin-7-yl)}-2-hydroxyacetamide;
N-{3-[(3-fluorophenyl)methyl]-4-oxo(3-hydroquinazolin-7-yl)}(phenylmethoxy)carboxamide;
N-{3-[(3,4-dichlorophenyl)methyl]-4-oxo(3-hydroquinazolin-6-yl)}(4-chlorophenoxy)carboxamide;
N-{3-[(3,4-dichlorophenyl)methyl]-4-oxo(3-hydroquinazolin-6-yl)}(2-phenylethoxy)carboxamide;
(N-{3-[(3-fluorophenyl)methyl]-4-oxo-3-hydroquinazolin-7-yl}carbamoyl)methyl acetate;
N-{3-[(3-chloro-4-fluorophenyl)methyl]-4-oxo(3-hydroquinazolin-7-yl)}(phenylmethoxy)carboxamide;
N-{3-[(3-chloro-4-fluorophenyl)methyl]-4-oxo(3-hydroquinazolin-7-yl)}(3-pyridylmethoxy)carboxamide;
(N-{3-[(3-chloro-4-fluorophenyl)methyl]-4-oxo-3-hydroquinazolin-7-yl}carbamoyl)methyl acetate;
N-{3-[(3-fluorophenyl)methyl]-4-oxo(3-hydroquinazolin-7-yl)}(3-pyridylmethoxy)carboxamide;
N-{3-[(4-chloro-3-fluorophenyl)methyl]-4-oxo(3-hydroquinazolin-7-yl)}(3-pyridylmethoxy)carboxamide;
(N-{3-[(4-chloro-3-fluorophenyl)methyl]-4-oxo-3-hydroquinazolin-7-yl}carbamoyl)methyl acetate;
N-{3-[(3,4-dichlorophenyl)methyl]-4-oxo(3-hydroquinazolin-6-yl)}(4-pyridylmethoxy)carboxamide;
N-{3-[(3,4-dichlorophenyl)methyl]-4-oxo(3-hydroquinazolin-6-yl)}[(3-cyanophenyl)methoxy]carboxamide;
N-{3-[(4-chlorophenyl)methyl]-4-oxo(3-hydroquinazolin-7-yl)}-2-hydroxyacetamide;
N-{3-[(3,4-dichlorophenyl)methyl]-4-oxo(3-hydroquinazolin-7-yl)}-2-hydroxyacetamide;
N-{3-[(4-chloro-3-fluorophenyl)methyl]-4-oxo(3-hydroquinazolin-7-yl)}(phenylmethoxy)carboxamide;
N-{3-[(3,4-dichlorophenyl)methyl]-4-oxo(3-hydroquinazolin-6-yl)}{2-[methylbenzylamino]ethoxy}carboxamide;
N-{3-[(3,4-dichlorophenyl)methyl]-4-oxo(3-hydroquinazolin-6-yl)}(cyclopropylmethoxy)carboxamide;
(N-{3-[(3,4-dichlorophenyl)methyl]-4-oxo-3-hydroquinazolin-6-yl}carbamoyl)methyl 2-(dimethylamino)acetate;
N-{3-[(3-chloro-4-fluorophenyl)methyl]-4-oxo(3-hydroquinazolin-7-yl)}-2-hydroxyacetamide;
ethyl 2-(N-{3-[(3,4-dichlorophenyl)methyl]-4-oxo-3-hydroquinazolin-6-yl}carbamoyloxy)acetate;
N-{3-[(3,4-dichlorophenyl)methyl]-4-oxo(3-hydroquinazolin-6-yl)}(2-pyridylmethoxy)carboxamide;
(N-{3-[(3,4-dichlorophenyl)methyl]-4-oxo-3-hydroquinazolin-7-yl}carbamoyl)methyl 2-(dimethylamino)acetate;
ethyl 2-(N-{3-[(3,4-dichlorophenyl)methyl]-4-oxo-3-hydroquinazolin-7-yl}carbamoyloxy)acetate;
N-{3-[(3,4-dichlorophenyl)methyl]-4-oxo(3-hydroquinazolin-6-yl)}[2-(2-methoxyethoxy)ethoxy]carboxamide;
N-{3-[(3,4-dichlorophenyl)methyl]-4-oxo(3-hydroquinazolin-6-yl)}[(2-methoxyethyl)amino]carboxamide;
[N-(4-oxo-3-{[3-(trifluoromethyl)phenyl]methyl}-3-hydroquinazolin-6-yl)carbamoyl]methyl acetate;
2-hydroxy-N-(4-oxo-3-{[3-(trifluoromethyl)phenyl]methyl}(3-hydroquinazolin-6-yl))acetamide;
[N-(4-oxo-3-{[4-(trifluoromethyl)phenyl]methyl}-3-hydroquinazolin-6-yl)carbamoyl]methyl acetate;
2-hydroxy-N-(4-oxo-3-{[4-(trifluoromethyl)phenyl]methyl}(3-hydroquinazolin-6-yl))acetamide;
(N-{3-[(4-chloro-3-fluorophenyl)methyl]-4-oxo-3-hydroquinazolin-6-yl}carbamoyl)methyl acetate;
N-{3-[(4-chloro-3-fluorophenyl)methyl]-4-oxo(3-hydroquinazolin-6-yl)}-2-hydroxyacetamide;
(N-{3-[(4-methoxyphenyl)methyl]-4-oxo-3-hydroquinazolin-6-yl}carbamoyl)methyl acetate;
2-hydroxy-N-{3-[(4-methoxyphenyl)methyl]-4-oxo(3-hydroquinazolin-6-yl)}acetamide;
(N-{3-[(3,4-difluorophenyl)methyl]-4-oxo-3-hydroquinazolin-7-yl}carbamoyl)methyl acetate;
N-{3-[(3,4-difluorophenyl)methyl]-4-oxo(3-hydroquinazolin-7-yl)}-2-hydroxyacetamide;
2-(3-(3,4-difluorobenzyl)-4-oxo-3,4-dihydroquinazolin-6-ylamino)-2-oxoethyl acetate;
N-(3-(3,4-difluorobenzyl)-4-oxo-3,4-dihydroquinazolin-6-yl)-2-hydroxyacetamide;
2-(3-(4-chlorobenzyl)-4-oxo-3,4-dihydroquinazolin-6-ylamino)-2-oxoethyl acetate;
N-(3-(4-chlorobenzyl)-4-oxo-3,4-dihydroquinazolin-6-yl)-2-hydroxyacetamide;
N-(3-(4-chlorobenzyl)-4-oxo-3,4-dihydroquinazolin-6-yl)-2-phenoxyacetamide;
2-(3-(4-chloro-3-(trifluoromethyl)benzyl)-4-oxo-3,4-dihydroquinazolin-6-ylamino)-2-oxoethyl acetate;
N-(3-(4-chloro-3-(trifluoromethyl)benzyl)-4-oxo-3,4-dihydroquinazolin-6-yl)-2-hydroxyacetamide;
benzyl 3-(3,4-dichlorobenzyl)-2-methyl-4-oxo-3,4-dihydroquinazolin-7-ylcarbamate;
benzyl 3-(1-(3,4-dichlorophenyl)ethyl)-4-oxo-3,4-dihydroquinazolin-7-ylcarbamate;
2-(3-(1-(3,4-dichlorophenyl)ethyl)-4-oxo-3,4-dihydroquinazolin-7-ylamino)-2-oxoethyl acetate;
N-(3-(1-(3,4-dichlorophenyl)ethyl)-4-oxo-3,4-dihydroquinazolin-7-yl)-2-hydroxyacetamide;
benzyl 3-(3-methoxybenzyl)-4-oxo-3,4-dihydroquinazolin-6-ylcarbamate;
2-(3-(3-chloro-4-fluorobenzyl)-4-oxo-3,4-dihydroquinazolin-6-ylamino)-2-oxoethyl acetate;
benzyl 3-(4-fluoro-3-(trifluoromethyl)benzyl)-4-oxo-3,4-dihydroquinazolin-6-ylcarbamate;
2-(3-(4-fluoro-3-(trifluoromethyl)benzyl)-4-oxo-3,4-dihydroquinazolin-6-ylamino)-2-oxoethyl acetate;
N-(3-(4-fluoro-3-(trifluoromethyl)benzyl)-4-oxo-3,4-dihydroquinazolin-6-yl)-2-hydroxyacetamide;
N-(3-(3,4-dichlorobenzyl)-4-oxo-3,4-dihydroquinazolin-6-yl)acetamide;
2-hydroxy-N-(4-oxo-3-(3-phenoxyphenyl)-3,4-dihydroquinazolin-6-yl)acetamide;
N-(3-(3,4-difluorobenzyl)-4-oxo-3,4-dihydroquinazolin-6-yl)acetamide;
N-(4-oxo-3-(4-(trifluoromethyl)benzyl)-3,4-dihydroquinazolin-6-yl)acetamide;
benzyl 3-(3,4-dimethylbenzyl)-4-oxo-3,4-dihydroquinazolin-6-ylcarbamate;
2-(3-(3,4-dimethylbenzyl)-4-oxo-3,4-dihydroquinazolin-6-ylamino)-2-oxoethyl acetate;
N-(3-(3,4-dimethylbenzyl)-4-oxo-3,4-dihydroquinazolin-6-yl)-2-hydroxyacetamide;
N-(3-(1-(3,4-dichlorophenyl)ethyl)-4-oxo-3,4-dihydroquinazolin-6-yl)-2-hydroxyacetamide;
2-oxo-2-(4-oxo-3-(3-phenylpropyl)-3,4-dihydroquinazolin-6-ylamino)ethyl acetate;
2-(3-(1-(3,4-dichlorophenyl)propyl)-4-oxo-3,4-dihydroquinazolin-6-ylamino)-2-oxoethyl acetate;
N-(3-(1-(3,4-dichlorophenyl)propyl)-4-oxo-3,4-dihydroquinazolin-6-yl)-2-hydroxyacetamide;
N-(3-benzyl-4-oxo-3,4-dihydroquinazolin-6-yl)-2-hydroxyacetamide;
2-hydroxy-N-(4-oxo-3-(3-phenylpropyl)-3,4-dihydroquinazolin-6-yl)acetamide;
2-hydroxy-N-(4-oxo-3-phenethyl-3,4-dihydroquinazolin-6-yl)acetamide;
2-oxo-2-(4-oxo-3-(2-(trifluoromethyl)benzyl)-3,4-dihydroquinazolin-6-ylamino)ethyl acetate;
N-(3-(biphenyl-3-ylmethyl)-4-oxo-3,4-dihydroquinazolin-6-yl)-2-hydroxyacetamide;
benzyl 3-(1-(3,4-dichlorophenyl)propyl)-4-oxo-3,4-dihydroquinazolin-6-ylcarbamate;
2-hydroxy-N-(3-(naphthalen-2-ylmethyl)-4-oxo-3,4-dihydroquinazolin-6-yl)acetamide;
benzyl 3-(naphthalen-2-ylmethyl)-4-oxo-3,4-dihydroquinazolin-6-ylcarbamate;
2-hydroxy-N-(3-(naphthalen-2-ylmethyl)-4-oxo-3,4-dihydroquinazolin-7-yl)acetamide;
(R)—N-(3-(1-(4-chloro-3-(trifluoromethyl)phenyl)ethyl)-4-oxo-3,4-dihydroquinazolin-6-yl)-2-hydroxyacetamide;
(S)—N-(3-(1-(3,4-dichlorophenyl)ethyl)-4-oxo-3,4-dihydroquinazolin-7-yl)-2-hydroxyacetamide;
(R)—N-(3-(1-(3,4-dichlorophenyl)ethyl)-4-oxo-3,4-dihydroquinazolin-7-yl)-2-hydroxyacetamide;
N-(3-(4-chlorophenethyl)-4-oxo-3,4-dihydroquinazolin-6-yl)-2-hydroxyacetamide;
N-(3-(3-chlorophenethyl)-4-oxo-3,4-dihydroquinazolin-6-yl)-2-hydroxyacetamide;
N-(3-(2,4-dichlorophenethyl)-4-oxo-3,4-dihydroquinazolin-6-yl)-2-hydroxyacetamide;
benzyl 4-oxo-3-(3-(trifluoromethyl)benzyl)-3,4-dihydroquinazolin-6-ylcarbamate;
benzyl 4-oxo-3-(3-(2-(trifluoromethyl)phenoxy)propyl)-3,4-dihydroquinazolin-6-ylcarbamate;
N-(3-(2-(2,5-dichlorophenoxy)ethyl)-4-oxo-3,4-dihydroquinazolin-6-yl)-2-hydroxyacetamide;
N-(3-(3-(2,5-dichlorophenoxy)propyl)-4-oxo-3,4-dihydroquinazolin-6-yl)-2-hydroxyacetamide;
benzyl 3-(3-(2,5-dichlorophenoxy)propyl)-4-oxo-3,4-dihydroquinazolin-6-ylcarbamate;
2-hydroxy-N-(4-oxo-3-(3-(2-(trifluoromethyl)phenoxy)propyl)-3,4-dihydroquinazolin-6-yl)acetamide;
benzyl 3-(3-(2-fluorophenoxy)propyl)-4-oxo-3,4-dihydroquinazolin-6-ylcarbamate;
2-(3-(3-(2-fluorophenoxy)propyl)-4-oxo-3,4-dihydroquinazolin-6-ylamino)-2-oxoethyl acetate;
N-(3-(3-(2-fluorophenoxy)propyl)-4-oxo-3,4-dihydroquinazolin-7-yl)-2-hydroxyacetamide;
benzyl 3-(3-(2-cyanophenoxy)propyl)-4-oxo-3,4-dihydroquinazolin-6-ylcarbamate;
benzyl 3-(3-(2,5-dichlorophenoxy)propyl)-4-oxo-3,4-dihydroquinazolin-7-ylcarbamate;
N-(3-(3-(2,5-dichlorophenoxy)propyl)-4-oxo-3,4-dihydroquinazolin-7-yl)-2-hydroxyacetamide;
benzyl 4-oxo-3-(3-(o-tolyloxy)propyl)-3,4-dihydroquinazolin-6-ylcarbamate;
2-hydroxy-N-(4-oxo-3-(3-(o-tolyloxy)propyl)-3,4-dihydroquinazolin-6-yl)acetamide;
benzyl 3-(4-(2,5-dichlorophenoxy)butyl)-4-oxo-3,4-dihydroquinazolin-6-ylcarbamate;
N-(3-(4-(2,5-dichlorophenoxy)butyl)-4-oxo-3,4-dihydroquinazolin-6-yl)-2-hydroxyacetamide;
N-(3-(3-(2-chlorophenyl)propyl)-4-oxo-3,4-dihydroquinazolin-6-yl)-2-hydroxyacetamide;
benzyl 3-(2-(2-chlorophenoxy)ethyl)-4-oxo-3,4-dihydroquinazolin-6-ylcarbamate;
N-(3-(2-(2-chlorophenoxy)ethyl)-4-oxo-3,4-dihydroquinazolin-6-yl)-2-hydroxyacetamide;
benzyl 3-(3-(2-chlorophenoxy)propyl)-4-oxo-3,4-dihydroquinazolin-6-ylcarbamate;
N-(3-(3-(2-chlorophenoxy)propyl)-4-oxo-3,4-dihydroquinazolin-6-yl)-2-hydroxyacetamide;
N-(3-(3-((2,5-dichlorophenoxy)methyl)benzyl)-4-oxo-3,4-dihydroquinazolin-6-yl)-2-hydroxyacetamide;
benzyl 3-(3-((2,5-dichlorophenoxy)methyl)benzyl)-4-oxo-3,4-dihydroquinazolin-6-ylcarbamate;
N-(3-(2-(2-chlorophenoxy)ethyl)-4-oxo-3,4-dihydroquinazolin-7-yl)-2-hydroxyacetamide;
N-(3-(5-(2,5-dichlorophenoxy)pentyl)-4-oxo-3,4-dihydroquinazolin-6-yl)-2-hydroxyacetamide;
N-(3-(3-(2,3-dichlorophenoxy)propyl)-4-oxo-3,4-dihydroquinazolin-6-yl)-2-hydroxyacetamide;
N-(3-(3-(2-chloro-5-(trifluoromethyl)phenoxy)propyl)-4-oxo-3,4-dihydroquinazolin-6-yl)-2-hydroxyacetamide;
2,5-dichloro-N-(2-(6-(2-hydroxyacetamido)-4-oxoquinazolin-3(4H)-yl)ethyl)benzamide;
2-hydroxy-N-(4-oxo-3-(3-phenoxypropyl)-3,4-dihydroquinazolin-6-yl)acetamide;
N-(2-(6-(2-hydroxyacetamido)-4-oxoquinazolin-3(4H)-yl)ethyl)-2-(trifluoromethyl)benzamide;
N-(3-(3-(4-chlorophenoxy)propyl)-4-oxo-3,4-dihydroquinazolin-6-yl)-2-hydroxyacetamide;
N-(3-(2-(2,5-dichlorophenylsulfonamido)ethyl)-4-oxo-3,4-dihydroquinazolin-6-yl)-2-hydroxyacetamide;
N-(3-(3-(2,5-dichlorophenylsulfonamido)propyl)-4-oxo-3,4-dihydroquinazolin-6-yl)-2-hydroxyacetamide;
2-hydroxy-N-(4-oxo-3-(3-(2-(trifluoromethyl)phenylsulfonamido)propyl)-3,4-dihydroquinazolin-6-yl)acetamide;
N-(3-(2-(4-chlorophenoxy)ethyl)-4-oxo-3,4-dihydroquinazolin-6-yl)-2-hydroxyacetamide;
N-(3-(2-(3-chlorophenoxy)ethyl)-4-oxo-3,4-dihydroquinazolin-6-yl)-2-hydroxyacetamide;
2-hydroxy-N-(3-(2-hydroxy-3-(o-tolyloxy)propyl)-4-oxo-3,4-dihydroquinazolin-6-yl)acetamide;
benzyl 3-(3,4-dichlorobenzyl)-4-oxo-3,4-dihydropyrido[2,3-d]pyrimidin-6-ylcarbamate;
N-(3-(3,4-dichlorobenzyl)-4-oxo-3,4-dihydropyrido[2,3-d]pyrimidin-6-yl)-2-phenoxyacetamide;
2-(3-(3,4-dichlorobenzyl)-4-oxo-3,4-dihydropyrido[2,3-d]pyrimidin-6-ylamino)-2-oxoethyl acetate;
N-(3-(3,4-dichlorobenzyl)-4-oxo-3,4-dihydropyrido[2,3-d]pyrimidin-6-yl)-2-hydroxyacetamide;
N-(3-(4-chlorobenzyl)-4-oxo-3,4-dihydropyrido[2,3-d]pyrimidin-6-yl)-2-hydroxyacetamide; and
benzyl 3-(4-chlorobenzyl)-4-oxo-3,4-dihydropyrido[2,3-d]pyrimidin-6-ylcarbamate.

### DETAILED DESCRIPTION OF THE INVENTION

### Definitions and General Parameters

As used in the present specification, the following words and phrases are generally intended to have the meanings as set forth below, except to the extent that the context in which they are used indicates otherwise.

The term “alkyl” refers to a monoradical branched or unbranched saturated hydrocarbon chain having 1, 2, 3, 4, 5, 6, 7, 8, 9, 10, 11, 12, 13, 14, 15, 16, 17, 18, 19 or 20 carbon atoms. This term is exemplified by groups such as methyl, ethyl, n-propyl, iso-propyl, n-butyl, iso-butyl, t-butyl, n-hexyl, n-decyl, tetradecyl, and the like.

The term “substituted alkyl” refers to:
1) an alkyl group as defined above, having 1, 2, 3, 4 or 5 substituents, preferably 1 to 3 substituents, selected from the group consisting of alkenyl, alkynyl, alkoxy, cycloalkyl, cycloalkenyl, acyl, acylamino, acyloxy, amino, aminocarbonyl, alkoxycarbonylamino, azido, cyano, halogen, hydroxy, keto, thiocarbonyl, carboxy, carboxyalkyl, arylthio, heteroarylthio, heterocyclylthio, thiol, alkylthio, aryl, aryloxy, heteroaryl, aminosulfonyl, aminocarbonylamino, heteroaryloxy, heterocyclyl, heterocyclooxy, hydroxyamino, alkoxyamino, nitro, —SO-alkyl, —SO-aryl, —SO-heteroaryl, —SO₂-alkyl, SO₂-aryl and —SO₂-heteroaryl. Unless otherwise constrained by the definition, all substituents may optionally be further substituted by 1, 2, or 3 substituents chosen from alkyl, carboxy, carboxyalkyl, aminocarbonyl, hydroxy, alkoxy, halogen, CF₃, amino, substituted amino, cyano, and —S(O)ₙR, where R is alkyl, aryl, or heteroaryl and n is 0, 1 or 2; or
2) an alkyl group as defined above that is interrupted by 1-10 atoms independently chosen from oxygen, sulfur and NRₐ—, where Rₐ is chosen from hydrogen, alkyl, cycloalkyl, alkenyl, cycloalkenyl, alkynyl, aryl, heteroaryl and heterocyclyl. All substituents may be optionally further substituted by alkyl, alkoxy, halogen, CF₃, amino, substituted amino, cyano, or —S(O)ₙR, in which R is alkyl, aryl, or heteroaryl and n is 0, 1 or 2; or
3) an alkyl group as defined above that has both 1, 2, 3, 4 or 5 substituents as defined above and is also interrupted by 1-10 atoms as defined above.

The term “lower alkyl” refers to a monoradical branched or unbranched saturated hydrocarbon chain having 1, 2, 3, 4, 5, or 6 carbon atoms. This term is exemplified by groups such as methyl, ethyl, n-propyl, iso-propyl, n-butyl, iso-butyl, t-butyl, n-hexyl, and the like.

The term “substituted lower alkyl” refers to lower alkyl as defined above having 1 to 5 substituents, preferably 1, 2, or 3 substituents, as defined for substituted alkyl, or a lower alkyl group as defined above that is interrupted by 1, 2, 3, 4, or 5 atoms as defined for substituted alkyl, or a lower alkyl group as defined above that has both 1, 2, 3, 4 or 5 substituents as defined above and is also interrupted by 1, 2, 3, 4, or 5 atoms as defined above.

The term “alkylene” refers to a diradical of a branched or unbranched saturated hydrocarbon chain, having 1, 2, 3, 4, 5, 6, 7, 8, 9, 10, 11, 12, 13, 14, 15, 16, 17, 18, 19 or 20 carbon atoms, preferably 1-10 carbon atoms, more preferably 1, 2, 3, 4, 5 or 6 carbon atoms. This term is exemplified by groups such as methylene (—CH₂—), ethylene (—CH₂CH₂—), the propylene isomers (e.g., —CH₂CH₂CH₂— and —CH(CH₃)CH₂—) and the like.

The term “lower alkylene” refers to a diradical of a branched or unbranched saturated hydrocarbon chain, preferably having from 1, 2, 3, 4, 5, or 6 carbon atoms.

The term “lower alkylene” refers to a diradical of a branched or unbranched saturated hydrocarbon chain, preferably having from 1, 2, 3, 4, 5, or 6 carbon atoms.

The term “substituted alkylene” refers to:
(1) an alkylene group as defined above having 1, 2, 3, 4, or 5 substituents selected from the group consisting of alkyl, alkenyl, alkynyl, alkoxy, cycloalkyl, cycloalkenyl, acyl, acylamino, acyloxy, amino, aminocarbonyl, alkoxycarbonylamino, azido, cyano, halogen, hydroxy, keto, thiocarbonyl, carboxy, carboxyalkyl, arylthio, heteroarylthio, heterocyclylthio, thiol, alkylthio, aryl, aryloxy, heteroaryl, aminosulfonyl, aminocarbonylamino, heteroaryloxy, heterocyclyl, heterocyclooxy, hydroxyamino, alkoxyamino, nitro, —SO-alkyl, —SO-aryl, —SO-heteroaryl, —SO₂-alkyl, SO₂-aryl and —SO₂-heteroaryl. Unless otherwise constrained by the definition, all substituents may optionally be further substituted by 1, 2, or 3 substituents chosen from alkyl, carboxy, carboxyalkyl, aminocarbonyl, hydroxy, alkoxy, halogen, CF₃, amino, substituted amino, cyano, and —S(O)ₙR, where R is alkyl, aryl, or heteroaryl and n is 0, 1 or 2; or
(2) an alkylene group as defined above that is interrupted by 1-20 atoms independently chosen from oxygen, sulfur and NRₐ—, where Rₐ is chosen from hydrogen, optionally substituted alkyl, cycloalkyl, cycloalkenyl, aryl, heteroaryl and heterocycyl, or groups selected from carbonyl, carboxyester, carboxyamide and sulfonyl; or
(3) an alkylene group as defined above that has both 1, 2, 3, 4 or 5 substituents as defined above and is also interrupted by 1-20 atoms as defined above. Examples of substituted alkylenes are chloromethylene (—CH(Cl)—), aminoethylene (—CH(NH₂)CH₂—), methylaminoethylene (—CH(NHMe)CH₂—), 2-carboxypropylene isomers (—CH₂CH(CO₂H)CH₂—), ethoxyethyl (—CH₂CH₂O—CH₂CH₂—), ethylmethylaminoethyl (—CH₂CH₂N(CH₃)CH₂CH₂—), 1-ethoxy-2-(2-ethoxy-ethoxy)ethane (—CH₂CH₂O—CH₂CH₂—OCH₂CH₂—OCH₂CH₂—), and the like.

The term “aralkyl” refers to an aryl group covalently linked to an alkylene group, where aryl and alkylene are defined herein. “Optionally substituted aralkyl” refers to an optionally substituted aryl group covalently linked to an optionally substituted alkylene group. Such aralkyl groups are exemplified by benzyl, phenylethyl, 3-(4-methoxyphenyl)propyl, and the like.

The term “alkoxy” refers to the group R—O—, where R is optionally substituted alkyl or optionally substituted cycloalkyl, or R is a group —Y-Z, in which Y is optionally substituted alkylene and Z is optionally substituted alkenyl, optionally substituted alkynyl; or optionally substituted cycloalkenyl, where alkyl, alkenyl, alkynyl, cycloalkyl and cycloalkenyl are as defined herein. Preferred alkoxy groups are optionally substituted alkyl-O— and include, by way of example, methoxy, ethoxy, n-propoxy, iso-propoxy, n-butoxy, tert-butoxy, sec-butoxy, n-pentoxy, n-hexoxy, 1,2-dimethylbutoxy, trifluoromethoxy, and the like.

The term “alkylthio” refers to the group R—S—, where R is as defined for alkoxy.

The term “alkenyl” refers to a monoradical of a branched or unbranched unsaturated hydrocarbon group preferably having from 2 to 20 carbon atoms, more preferably 2 to 10 carbon atoms and even more preferably 2 to 6 carbon atoms and having 1-6, preferably 1, double bond (vinyl). Preferred alkenyl groups include ethenyl or vinyl (—CH═CH₂), 1-propylene or allyl (—CH₂CH═CH₂), isopropylene (—C(CH₃)═CH₂), bicyclo[2.2.1]heptene, and the like. In the event that alkenyl is attached to nitrogen, the double bond cannot be alpha to the nitrogen.

The term “lower alkenyl” refers to alkenyl as defined above having from 2 to 6 carbon atoms.

The term “substituted alkenyl” refers to an alkenyl group as defined above having 1, 2, 3, 4 or 5 substituents, and preferably 1, 2, or 3 substituents, selected from the group consisting of alkyl, alkenyl, alkynyl, alkoxy, cycloalkyl, cycloalkenyl, acyl, acylamino, acyloxy, amino, aminocarbonyl, alkoxycarbonylamino, azido, cyano, halogen, hydroxy, keto, thiocarbonyl, carboxy, carboxyalkyl, arylthio, heteroarylthio, heterocyclylthio, thiol, alkylthio, aryl, aryloxy, heteroaryl, aminosulfonyl, aminocarbonylamino, heteroaryloxy, heterocyclyl, heterocyclooxy, hydroxyamino, alkoxyamino, nitro, —SO-alkyl, —SO-aryl, —SO-heteroaryl, —SO₂-alkyl, SO₂-aryl and —SO₂-heteroaryl. Unless otherwise constrained by the definition, all substituents may optionally be further substituted by 1, 2, or 3 substituents chosen from alkyl, carboxy, carboxyalkyl, aminocarbonyl, hydroxy, alkoxy, halogen, CF₃, amino, substituted amino, cyano, and —S(O)ₙR, where R is alkyl, aryl, or heteroaryl and n is 0, 1 or 2.

The term “alkynyl” refers to a monoradical of an unsaturated hydrocarbon, preferably having from 2 to 20 carbon atoms, more preferably 2 to 10 carbon atoms and even more preferably 2 to 6 carbon atoms and having at least 1 and preferably from 1-6 sites of acetylene (triple bond) unsaturation. Preferred alkynyl groups include ethynyl, (—C≡CH), propargyl (or prop-1-yn-3-yl, —CH₂C≡CH), and the like. In the event that alkynyl is attached to nitrogen, the triple bond cannot be alpha to the nitrogen.

The term “substituted alkynyl” refers to an alkynyl group as defined above having 1, 2, 3, 4 or 5 substituents, and preferably 1, 2, or 3 substituents, selected from the group consisting of alkyl, alkenyl, alkynyl, alkoxy, cycloalkyl, cycloalkenyl, acyl, acylamino, acyloxy, amino, aminocarbonyl, alkoxycarbonylamino, azido, cyano, halogen, hydroxy, keto, thiocarbonyl, carboxy, carboxyalkyl, arylthio, heteroarylthio, heterocyclylthio, thiol, alkylthio, aryl, aryloxy, heteroaryl, aminosulfonyl, aminocarbonylamino, heteroaryloxy, heterocyclyl, heterocyclooxy, hydroxyamino, alkoxyamino, nitro, —SO-alkyl, —SO-aryl, —SO-heteroaryl, —SO₂-alkyl, SO₂-aryl and —SO₂-heteroaryl. Unless otherwise constrained by the definition, all substituents may optionally be further substituted by 1, 2, or 3 substituents chosen from alkyl, carboxy, carboxyalkyl, aminocarbonyl, hydroxy, alkoxy, halogen, CF₃, amino, substituted amino, cyano, and —S(O)ₙR, where R is alkyl, aryl, or heteroaryl and n is 0, 1 or 2.

The term “aminocarbonyl” refers to the group —C(O)NRR where each R is independently hydrogen, alkyl, aryl, heteroaryl, heterocyclyl or where both R groups are joined to form a heterocyclic group (e.g., morpholino). Unless otherwise constrained by the definition, all substituents may optionally be further substituted by 1-3 substituents chosen from alkyl, carboxy, carboxyalkyl, aminocarbonyl, hydroxy, alkoxy, halogen, CF₃, amino, substituted amino, cyano, and —S(O)ₙR, where R is alkyl, aryl, or heteroaryl and n is 0, 1 or 2.

The term “acylamino” refers to the group —NRC(O)R where each R is independently hydrogen, alkyl, aryl, heteroaryl, or heterocyclyl. Unless otherwise constrained by the definition, all substituents may optionally be further substituted by 1-3 substituents chosen from alkyl, carboxy, carboxyalkyl, aminocarbonyl, hydroxy, alkoxy, halogen, CF₃, amino, substituted amino, cyano, and —S(O)ₙR, where R is alkyl, aryl, or heteroaryl and n is 0, 1 or 2.

The term “acyloxy” refers to the groups —O(O)C-alkyl, —O(O)C-cycloalkyl, —O(O)C-aryl, —O(O)C-heteroaryl, and —O(O)C-heterocyclyl. Unless otherwise constrained by the definition, all substituents may be optionally further substituted by alkyl, carboxy, carboxyalkyl, aminocarbonyl, hydroxy, alkoxy, halogen, CF₃, amino, substituted amino, cyano, or —S(O)ₙR, where R is alkyl, aryl, or heteroaryl and n is 0, 1 or 2.

The term “aryl” refers to an aromatic carbocyclic group of 6 to 20 carbon atoms having a single ring (e.g., phenyl) or multiple rings (e.g., biphenyl), or multiple condensed (fused) rings (e.g., naphthyl or anthryl). Preferred aryls include phenyl, naphthyl and the like.

The term “arylene” refers to a diradical of an aryl group as defined above. This term is exemplified by groups such as 1,4-phenylene, 1,3-phenylene, 1,2-phenylene, 1,4′-biphenylene, and the like.

Unless otherwise constrained by the definition for the aryl or arylene substituent, such aryl or arylene groups can optionally be substituted with from 1 to 5 substituents, preferably 1 to 3 substituents, selected from the group consisting of alkyl, alkenyl, alkynyl, alkoxy, cycloalkyl, cycloalkenyl, acyl, acylamino, acyloxy, amino, aminocarbonyl, alkoxycarbonylamino, azido, cyano, halogen, hydroxy, keto, thiocarbonyl, carboxy, carboxyalkyl, arylthio, heteroarylthio, heterocyclylthio, thiol, alkylthio, aryl, aryloxy, heteroaryl, aminosulfonyl, aminocarbonylamino, heteroaryloxy, heterocyclyl, heterocyclooxy, hydroxyamino, alkoxyamino, nitro, —SO-alkyl, —SO-aryl, —SO-heteroaryl, —SO₂-alkyl, SO₂-aryl and —SO₂— heteroaryl. Unless otherwise constrained by the definition, all substituents may optionally be further substituted by 1-3 substituents chosen from alkyl, carboxy, carboxyalkyl, aminocarbonyl, hydroxy, alkoxy, halogen, CF₃, amino, substituted amino, cyano, and —S(O)ₙR, where R is alkyl, aryl, or heteroaryl and n is 0, 1 or 2.

The term “aryloxy” refers to the group aryl-O— wherein the aryl group is as defined above, and includes optionally substituted aryl groups as also defined above. The term “arylthio” refers to the group R—S—, where R is as defined for aryl.

The term “amino” refers to the group —NH₂.

The term “substituted amino” refers to the group —NRR where each R is independently selected from the group consisting of hydrogen, alkyl, cycloalkyl, carboxyalkyl (for example, benzyloxycarbonyl), aryl, heteroaryl and heterocyclyl provided that both R groups are not hydrogen, or a group —Y-Z, in which Y is optionally substituted alkylene and Z is alkenyl, cycloalkenyl, or alkynyl, Unless otherwise constrained by the definition, all substituents may optionally be further substituted by 1-3 substituents chosen from alkyl, carboxy, carboxyalkyl, aminocarbonyl, hydroxy, alkoxy, halogen, CF₃, amino, substituted amino, cyano, and —S(O)ₙR, where R is alkyl, aryl, or heteroaryl and n is 0, 1 or 2.

The term “carboxyalkyl” refers to the groups —C(O)O-alkyl or —C(O)O-cycloalkyl, where alkyl and cycloalkyl, are as defined herein, and may be optionally further substituted by alkyl, alkenyl, alkynyl, alkoxy, halogen, CF₃, amino, substituted amino, cyano, or —S(O)ₙR, in which R is alkyl, aryl, or heteroaryl and n is 0, 1 or 2.

The term “cycloalkyl” refers to carbocyclic groups of from 3 to 20 carbon atoms having a single cyclic ring or multiple condensed rings. Such cycloalkyl groups include, by way of example, single ring structures such as cyclopropyl, cyclobutyl, cyclopentyl, cyclooctyl, and the like, or multiple ring structures such as adamantanyl, bicyclo[2.2.1]heptane, 1,3,3-trimethylbicyclo[2.2.1]hept-2-yl, (2,3,3-trimethylbicyclo[2.2.1]hept-2-yl), or carbocyclic groups to which is fused an aryl group, for example indane, and the like.

The term “substituted cycloalkyl” refers to cycloalkyl groups having 1, 2, 3, 4 or 5 substituents, and preferably 1, 2, or 3 substituents, selected from the group consisting of alkyl, alkenyl, alkynyl, alkoxy, cycloalkyl, cycloalkenyl, acyl, acylamino, acyloxy, amino, aminocarbonyl, alkoxycarbonylamino, azido, cyano, halogen, hydroxy, keto, thiocarbonyl, carboxy, carboxyalkyl, arylthio, heteroarylthio, heterocyclylthio, thiol, alkylthio, aryl, aryloxy, heteroaryl, aminosulfonyl, aminocarbonylamino, heteroaryloxy, heterocyclyl, heterocyclooxy, hydroxyamino, alkoxyamino, nitro, —SO-alkyl, —SO-aryl, —SO-heteroaryl, —SO₂-alkyl, SO₂-aryl and —SO₂-heteroaryl. Unless otherwise constrained by the definition, all substituents may optionally be further substituted by 1, 2, or 3 substituents chosen from alkyl, carboxy, carboxyalkyl, aminocarbonyl, hydroxy, alkoxy, halogen, CF₃, amino, substituted amino, cyano, and —S(O)ₙR, where R is alkyl, aryl, or heteroaryl and n is 0, 1 or 2.

The term “halogen” or “halo” refers to fluoro, bromo, chloro, and iodo.

The term “acyl” denotes a group —C(O)R, in which R is hydrogen, optionally substituted alkyl, optionally substituted cycloalkyl, optionally substituted heterocyclyl, optionally substituted aryl, and optionally substituted heteroaryl.

The term “heteroaryl” refers to a radical derived from an aromatic cyclic group (i.e., fully unsaturated) having 1, 2, 3, 4, 5, 6, 7, 8, 9, 10, 11, 12, 13, 14, or 15 carbon atoms and 1, 2, 3 or 4 heteroatoms selected from oxygen, nitrogen and sulfur within at least one ring. Such heteroaryl groups can have a single ring (e.g., pyridyl or furyl) or multiple condensed rings (e.g., indolizinyl, benzothiazolyl, or benzothienyl). Examples of heteroaryls include, but are not limited to, [1,2,4]oxadiazole, [1,3,4]oxadiazole, [1,2,4]thiadiazole, [1,3,4]thiadiazole, pyrrole, imidazole, pyrazole, pyridine, pyrazine, pyrimidine, pyridazine, indolizine, isoindole, indole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, and the like as well as N-oxide and N-alkoxy derivatives of nitrogen containing heteroaryl compounds, for example pyridine-N-oxide derivatives.

The term “heteroarylene” refers to a diradical of a heteroaryl group as defined above. This term is exemplified by groups such as 2,5-imidazolene, 3,5-[1,2,4]oxadiazolene, 2,4-oxazolene, 1,4-pyrazolene, and the like. For example, 1,4-pyrazolene is:

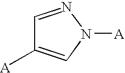

where A represents the point of attachment.

Unless otherwise constrained by the definition for the heteroaryl or heteroarylene substituent, such heteroaryl or heterarylene groups can be optionally substituted with 1 to 5 substituents, preferably 1 to 3 substituents selected from the group consisting of alkyl, alkenyl, alkynyl, alkoxy, cycloalkyl, cycloalkenyl, acyl, acylamino, acyloxy, amino, aminocarbonyl, alkoxycarbonylamino, azido, cyano, halogen, hydroxy, keto, thiocarbonyl, carboxy, carboxyalkyl, arylthio, heteroarylthio, heterocyclylthio, thiol, alkylthio, aryl, aryloxy, heteroaryl, aminosulfonyl, aminocarbonylamino, heteroaryloxy, heterocyclyl, heterocyclooxy, hydroxyamino, alkoxyamino, nitro, —SO-alkyl, —SO-aryl, —SO-heteroaryl, —SO₂-alkyl, SO₂-aryl and —SO₂-heteroaryl. Unless otherwise constrained by the definition, all substituents may optionally be further substituted by 1-3 substituents chosen from alkyl, carboxy, carboxyalkyl, aminocarbonyl, hydroxy, alkoxy, halogen, CF₃, amino, substituted amino, cyano, and —S(O)ₙR, where R is alkyl, aryl, or heteroaryl and n is 0, 1 or 2.

The term “heteroaralkyl” refers to a heteroaryl group covalently linked to an alkylene group, where heteroaryl and alkylene are defined herein. “Optionally substituted heteroaralkyl” refers to an optionally substituted heteroaryl group covalently linked to an optionally substituted alkylene group. Such heteroaralkyl groups are exemplified by 3-pyridylmethyl, quinolin-8-ylethyl, 4-methoxythiazol-2-ylpropyl, and the like.

The term “heteroaryloxy” refers to the group heteroaryl-O—.

The term “heterocyclyl” refers to a monoradical saturated or partially unsaturated group having a single ring or multiple condensed rings, having from 1 to 40 carbon atoms and from 1 to 10 hetero atoms, preferably 1, 2, 3 or 4 heteroatoms, selected from nitrogen, sulfur, phosphorus, and/or oxygen within the ring. Heterocyclic groups can have a single ring or multiple condensed rings, and include tetrahydrofuranyl, morpholino, piperidinyl, piperazino, dihydropyridino, and the like.

Unless otherwise constrained by the definition for the heterocyclic substituent, such heterocyclic groups can be optionally substituted with 1, 2, 3, 4 or 5, and preferably 1, 2 or 3 substituents, selected from the group consisting of alkyl, alkenyl, alkynyl, alkoxy, cycloalkyl, cycloalkenyl, acyl, acylamino, acyloxy, amino, aminocarbonyl, alkoxycarbonylamino, azido, cyano, halogen, hydroxy, keto, thiocarbonyl, carboxy, carboxyalkyl, arylthio, heteroarylthio, heterocyclylthio, thiol, alkylthio, aryl, aryloxy, heteroaryl, aminosulfonyl, aminocarbonylamino, heteroaryloxy, heterocyclyl, heterocyclooxy, hydroxyamino, alkoxyamino, nitro, —SO-alkyl, —SO-aryl, —SO-heteroaryl, —SO₂-alkyl, SO₂-aryl and —SO₂-heteroaryl. Unless otherwise constrained by the definition, all substituents may optionally be further substituted by 1-3 substituents chosen from alkyl, carboxy, carboxyalkyl, aminocarbonyl, hydroxy, alkoxy, halogen, CF₃, amino, substituted amino, cyano, and —S(O)ₙR, where R is alkyl, aryl, or heteroaryl and n is 0, 1 or 2.

The term “thiol” refers to the group —SH.

The term “substituted alkylthio” refers to the group —S-substituted alkyl.

The term “heteroarylthiol” refers to the group —S-heteroaryl wherein the heteroaryl group is as defined above including optionally substituted heteroaryl groups as also defined above.

The term “sulfoxide” refers to a group —S(O)R, in which R is alkyl, aryl, or heteroaryl.

“Substituted sulfoxide” refers to a group —S(O)R, in which R is substituted alkyl, substituted aryl, or substituted heteroaryl, as defined herein.

The term “sulfone” refers to a group —S(O)₂R, in which R is alkyl, aryl, or heteroaryl.

“Substituted sulfone” refers to a group —S(O)₂R, in which R is substituted alkyl, substituted aryl, or substituted heteroaryl, as defined herein.

The term “keto” refers to a group —C(O)—.

The term “thiocarbonyl” refers to a group —C(S)—.

The term “carboxy” refers to a group —C(O)—OH.

“Optional” or “optionally” means that the subsequently described event or circumstance may or may not occur, and that the description includes instances where said event or circumstance occurs and instances in which it does not.

The term “compound of Formula I” is intended to encompass the compounds of the invention as disclosed, and the pharmaceutically acceptable salts, pharmaceutically acceptable esters, prodrugs, hydrates and polymorphs of such compounds. Additionally, the compounds of the invention may possess one or more asymmetric centers, and can be produced as a racemic mixture or as individual enantiomers or diastereoisomers. The number of stereoisomers present in any given compound of Formula I depends upon the number of asymmetric centers present (there are 2ⁿ stereoisomers possible where n is the number of asymmetric centers). The individual stereoisomers may be obtained by resolving a racemic or non-racemic mixture of an intermediate at some appropriate stage of the synthesis, or by resolution of the compound of Formula I by conventional means. The individual stereoisomers (including individual enantiomers and diastereoisomers) as well as racemic and non-racemic mixtures of stereoisomers are encompassed within the scope of the present invention, all of which are intended to be depicted by the structures of this specification unless otherwise specifically indicated.

“Isomers” are different compounds that have the same molecular formula.

“Stereoisomers” are isomers that differ only in the way the atoms are arranged in space.

“Enantiomers” are a pair of stereoisomers that are non-superimposable mirror images of each other. A 1:1 mixture of a pair of enantiomers is a “racemic” mixture. The term “(±)” is used to designate a racemic mixture where appropriate.

“Diastereoisomers” are stereoisomers that have at least two asymmetric atoms, but which are not mirror-images of each other.

The absolute stereochemistry is specified according to the Cahn-Ingold-Prelog R-S system. When the compound is a pure enantiomer the stereochemistry at each chiral carbon may be specified by either R or S. Resolved compounds whose absolute configuration is unknown are designated (+) or (−) depending on the direction (dextro- or laevorotary) which they rotate the plane of polarized light at the wavelength of the sodium D line.

“Parenteral administration” is the systemic delivery of the therapeutic agent via injection to the patient.

The term “therapeutically effective amount” refers to that amount of a compound of Formula I that is sufficient to effect treatment, as defined below, when administered to a mammal in need of such treatment. The therapeutically effective amount will vary depending upon the specific activity of the therapeutic agent being used, and the age, physical condition, existence of other disease states, and nutritional status of the patient. Additionally, other medication the patient may be receiving will effect the determination of the therapeutically effective amount of the therapeutic agent to administer.

The term “treatment” or “treating” means any treatment of a disease in a mammal, including:

(i) preventing the disease, that is, causing the clinical symptoms of the disease not to develop;

(ii) inhibiting the disease, that is, arresting the development of clinical symptoms; and/or

(iii) relieving the disease, that is, causing the regression of clinical symptoms.

In many cases, the compounds of this invention are capable of forming acid and/or base salts by virtue of the presence of amino and/or carboxyl groups or groups similar thereto. The term “pharmaceutically acceptable salt” refers to salts that retain the biological effectiveness and properties of the compounds of Formula I and which are not biologically or otherwise undesirable. Pharmaceutically acceptable base addition salts can be prepared from inorganic and organic bases. Salts derived from inorganic bases, include by way of example only, sodium, potassium, lithium, ammonium, calcium and magnesium salts. Salts derived from organic bases include, but are not limited to, salts of primary, secondary and tertiary amines, such as alkyl amines, dialkyl amines, trialkyl amines, substituted alkyl amines, di(substituted alkyl) amines, tri(substituted alkyl) amines, alkenyl amines, dialkenyl amines, trialkenyl amines, substituted alkenyl amines, di(substituted alkenyl) amines, tri(substituted alkenyl) amines, cycloalkyl amines, di(cycloalkyl) amines, tri(cycloalkyl) amines, substituted cycloalkyl amines, disubstituted cycloalkyl amine, trisubstituted cycloalkyl amines, cycloalkenyl amines, di(cycloalkenyl) amines, tri(cycloalkenyl) amines, substituted cycloalkenyl amines, disubstituted cycloalkenyl amine, trisubstituted cycloalkenyl amines, aryl amines, diaryl amines, triaryl amines, heteroaryl amines, diheteroaryl amines, triheteroaryl amines, heterocyclic amines, diheterocyclic amines, triheterocyclic amines, mixed di- and tri-amines where at least two of the substituents on the amine are different and are selected from the group consisting of alkyl, substituted alkyl, alkenyl, substituted alkenyl, cycloalkyl, substituted cycloalkyl, cycloalkenyl, substituted cycloalkenyl, aryl, heteroaryl, heterocyclic, and the like. Also included are amines where the two or three substituents, together with the amino nitrogen, form a heterocyclic or heteroaryl group.

Specific examples of suitable amines include, by way of example only, isopropylamine, trimethyl amine, diethyl amine, tri(iso-propyl) amine, tri(n-propyl) amine, ethanolamine, 2-dimethylaminoethanol, tromethamine, lysine, arginine, histidine, caffeine, procaine, hydrabamine, choline, betaine, ethylenediamine, glucosamine, N-alkylglucamines, theobromine, purines, piperazine, piperidine, morpholine, N-ethylpiperidine, and the like.

Pharmaceutically acceptable acid addition salts may be prepared from inorganic and organic acids. Salts derived from inorganic acids include hydrochloric acid, hydrobromic acid, sulfuric acid, nitric acid, phosphoric acid, and the like. Salts derived from organic acids include acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, and the like.

As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions.

### Nomenclature

Names of compounds of the present invention are provided using ChemDraw Ultra v. 10.0 (CambridgeSoft, Cambridge, Mass.). Some compounds or radicals may be named with common names, or systematic or non-systematic names. The naming and numbering of the compounds of the invention is illustrated with a representative compound of Formula I in which R¹ is benzyl, R² and R³ are chloro, R⁴ and R⁵ are hydrogen, Y is methylene, and X is oxygen,

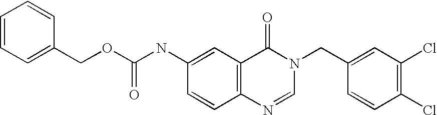

which is named, N-{3-[(3,4-dichlorophenyl)methyl]-4-oxo(3-hydroquinazolin-6-yl)}(phenylmethoxy)carboxamide.

### Pharmaceutical Compositions

When selected as the SCD inhibitor, the compounds of Formula I are usually administered in the form of pharmaceutical compositions. This invention therefore provides pharmaceutical compositions that contain, as the active ingredient, one or more of the compounds of Formula I, or a pharmaceutically acceptable salt or ester thereof, and one or more pharmaceutically acceptable excipients, carriers, including inert solid diluents and fillers, diluents, including sterile aqueous solution and various organic solvents, solubilizers and adjuvants. The compounds of Formula I may be administered alone or in combination with other therapeutic agents. Such compositions are prepared in a manner well known in the pharmaceutical art (see, e.g., Remington's Pharmaceutical Sciences, Mace Publishing Co., Philadelphia, Pa. 17^{th} Ed. (1985) and “Modern Pharmaceutics”, Marcel Dekker, Inc. 3^{rd} Ed. (G. S. Banker & C. T. Rhodes, Eds.).

### Synthetic Reaction Parameters

The terms “solvent”, “inert organic solvent” or “inert solvent” mean a solvent inert under the conditions of the reaction being described in conjunction therewith [including, for example, benzene, toluene, acetonitrile, tetrahydrofuran (“THF”), dimethylformamide (“DMF”), chloroform, methylene chloride (or dichloromethane), diethyl ether, methanol, pyridine and the like]. Unless specified to the contrary, the solvents used in the reactions of the present invention are inert organic solvents, and the reactions are carried out under an inert gas, preferably nitrogen.

The term “q.s.” means adding a quantity sufficient to achieve a stated function, e.g., to bring a solution to the desired volume (i.e., 100%).

### Synthesis of the Compounds of Formula I

One method of preparing compounds of Formula I wherein Q is —C(O)—NH— is shown in Reaction Scheme I.

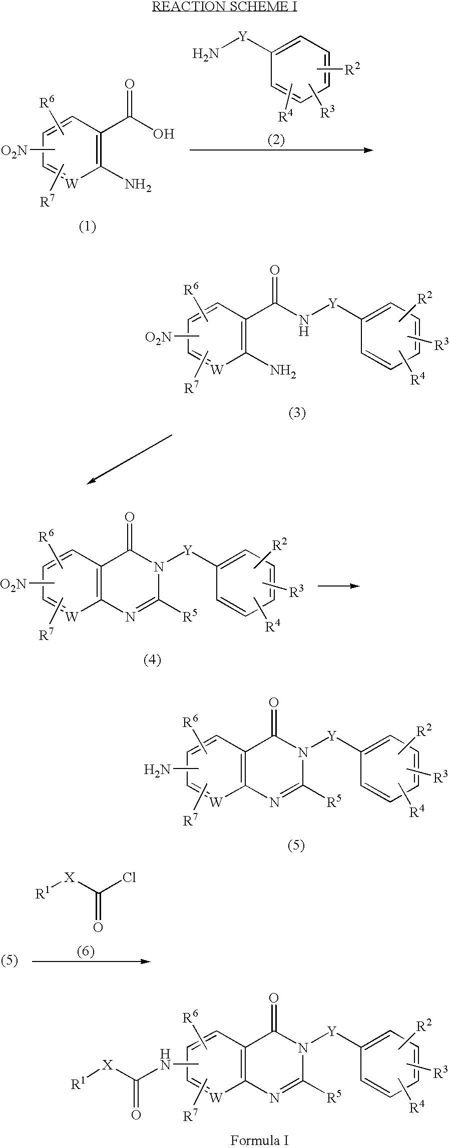

### Step 1—Preparation of Formula (3)

The compound of formula (3) is made by forming a peptide bond between the amino group on the commercially available compound of formula (2) and the acid moiety on the commercially available nitro substituted 2-amino benzoic or nicotinic acid compound of formula (1). The reaction takes place at room temperature and is typically conducted in a polar solvent such as dichloromethane in the presence of peptide coupling agents such as 1-Ethyl-3-(3-dimethylaminopropyl)carbodiimide (EDC) and 1-Hydroxybenzotriazole (HOBt) and may take from 2 to 5 hours. When the reaction is substantially complete, the product of formula (3) is isolated by conventional means, for example by organic phase separation using NaHCO₃ and NH₄Cl followed by removal of the solvent under reduced pressure and drying under high vacuum.

### Step 2—Preparation of Formula (4)

The compound of formula (4) is made by forming the quinazoline or pyrido[2,3-d]pyrimidin-4(3H)-one core via condensation with trimethylorthoformate or triethylorthoformate and (D)-10-camphorsulfinic acid. The reaction is subjected to microwave irradiation maintaining an internal reaction temperature of 160 to 200° C. for 20 minutes to an hour. Once the reaction is complete, the solvent may be removed by vacuum is typically used in the next step without purification.

### Step 3—Preparation of Formula (5)

The formula (4) nitro compound is then reduced to the corresponding amine analog, compound (5), via conventional reduction techniques. Suitable methods include, but are not limited to, dissolution in p-dioxane and reaction with Na₂S₂O₄ and Na₂CO₃ at room temperature for 12 to 24 hours. After the reaction is substantially complete, the product may be extracted by dilution with EtOAc followed by washing with saturated. NaHCO₃ solution and brine. The combined organic phase can then be dried over Na₂SO₄ and concentrated to provide the compound of formula (5) in crude form which can be used in the next step without further purification.

Alternatively, the nitro group can be reduced by reaction with hydrazine and a Raney-Nickel catalyst. In this method, the nitro compound is placed in a methanol solution to which the hydrazine is added. Then the reaction mixture is heated to approximately 50° C. to 80° C. and the Raney-Nickel catalyst gently added to insure even and steady evolution of the nitrogen gas. The reaction proceeds for approximately 1 hour whereupon the reaction mixture is allowed to cool to room temperature, the catalyst filtered off, and the filter cake washed with methanol. The resulting solution may be concentrated and purified using conventional methods, i.e., chromatography using a methanol/dichloromethane gradient to provide the desired amine.

### Step 4—Preparation of Formula I

The final step in the synthesis involves the addition of the R¹—XC(O)— portion of the compound. This is achieved by reacting the amino compound of formula (5) with an chloroformate, thiocarbonyl chloride, or alkanoyl chloride derivative having the desired R¹—XC(O)— moiety, i.e., a compound of formula (6). The compound of formula (5) is dissolved in p-dioxane and a solution of Na₂CO₃ in water is added. The compound of formula (6) is then added and the reaction is stirred at room temperature for approximately 1 to 24 hours. The mixture is then diluted with dichloromethane and more Na₂CO₃ solution. The resulting layers are separated in the organic phase dried with Na₂SO₄. The final product extracted by drying under vacuum to remove any remaining solvent followed by reverse-phase chromatography.

### Alternative Preparations of Formula I

In instances where X is nitrogen, one method of preparing the Compound of Formula I is by reacting the amino compound of formula (5) with an isocyanate derivative having the desired R¹-moiety, i.e., a compound of formula (6′). R¹—N═C═O. As before, the compound of formula (5) is dissolved in p-dioxane and a solution of Na₂CO₃ in water is added. The compound of formula (6′) is then added and the reaction is stirred at room temperature for approximately 1 to 24 hours. The mixture is then diluted with dichloromethane and more Na₂CO₃ solution. The resulting layers are separated in the organic phase and dried with Na₂SO₄. The final product is extracted by drying under vacuum to remove any remaining and purified via prep-TLC eluting with a methanol and dichloromethane solution.

Compounds of Formula I wherein R¹ is an acetoxy substituted alkyl can be synthesized by reaction of the compound of formula (5) with a solution containing acetylglycolic acid in methylene chloride. The reaction is stirred at approximately 50° C. to 80° C. for 1 to 4 hours. The compound of Formula I can them be collected and purified using conventional techniques such as solvent removal followed by column chromatography.

### Alternative Preparation—Secondary Modification of R¹

It will be appreciated that secondary modification may be made to the R¹ moiety after the compound of Formula I has been made. In one such case, a Formula I compound having a terminal acetoxy group can be reacted with a base such as LiOH in a polar solvent such as methanol to provide the analogous hydroxy derivative.

### Utility Testing and Administration

The present invention relates to compounds, pharmaceutical compositions and methods of using the compounds and pharmaceutical compositions for the treatment and/or prevention of diseases mediated by SCD. The methods and pharmaceutical compositions are particularly suitable for use in the treatment of diseases related to dyslipidemia and disorders of lipid metabolism, especially diseases related to elevated plasma and tissue lipid levels, such as cardiovascular disease, diabetes, obesity, metabolic syndrome, fatty liver diseases and the like.

In general, the compounds of the invention find utility in the treatment of a patient for, or protecting a patient from developing, a disease related to dyslipidemia and/or a disorder of lipid metabolism, wherein lipid levels in an animal, especially a human being, are outside the normal range (i.e., abnormal lipid level, such as elevated plasma or tissue lipid levels), preferably where said lipid is a fatty acid, such as a free or complexed fatty acid, triglycerides, phospholipids, wax esters, or cholesterol, such as where VLDL, hepatic or peripheral tissue triglycerides are elevated, or any combination of these, where said lipid-related condition or disease is an SCD-mediated disease or condition such as metabolic syndrome, diabetes, non-alcoholic fatty liver disease, obesity, cancer, oily skin and related diseases, comprising administering to an animal, such as a mammal, especially a human patient, a therapeutically effective amount of a compound of the invention or a pharmaceutical composition comprising a compound of the invention wherein the compound inhibits the activity of SCD.

The general value of the compounds of the invention in inhibiting the activity of SCD can be determined using the assay described below in Example 12. Additionally, the general value of the compounds in treating disorders and diseases may be established in industry standard animal models for demonstrating the efficacy of compounds in treating obesity, diabetes, metabolic syndrome or abnormal triglyceride or cholesterol levels or for improving glucose tolerance.

### Utility

The compounds of the instant invention are inhibitors of SCD and are useful for treating diseases and disorders in humans and other organisms, including all those human diseases and disorders which are the result of aberrant SCD biological activity or which may be ameliorated by inhibition of SCD biological activity.

As defined herein, an SCD-mediated disease or condition includes but is not limited to a disease or condition which is, or is related to, cardiovascular disease, dyslipidemias, coronary artery disease, atherosclerosis, heart disease, cerebrovascular disease (including, but not limited, to stroke, ischemic stroke and transient ischemic attack (TIA), peripheral vascular disease, and ischemic retinopathy, cancers and oily skin.

Dyslipidemia, as used herein, includes, but is not limited to, disorders related to the serum levels of triglycerides, i.e., hypertriglyceridemia, LDL, VLDL, and/or HDL, cholesterol, and total cholesterol. Dyslipidemia also includes disorders related to the fatty acid Desaturation Index (e.g. the ratio of SCD product fatty acids/SCD substrate fatty acids). Disorders relation polyunsaturated fatty acid (PUFA) are also included as are cholesterol disorders such as familial combined hyperlipidemia and those disordersinvolving defective reverse cholesterol transport.

SCD-mediated diseases or conditions relating to hypertriglyceridemia, include but are not limited to, hyperlipoproteinemias, familial histiocytic reticulosis, lipoprotein lipase deficiency, apolipoprotein deficiency (such as ApoCII deficiency or ApoE deficiency), and the like, or hypertriglyceridemia of unknown or unspecified etiology.

Metabolic syndrome and Syndrome X are also within the scope of the term “SCD-mediated disease” including all of the various component condition that make up the syndromes such as, but not limited to, dyslipidemia, low HDL, obesity, insulin resistance, decreased glucose tolerance, hypertension, microalbuminemia, hyperuricaemia, and hypercoagulability, diabetes, non-insulin-dependent diabetes mellitus, Type I diabetes, Type II diabetes, diabetic complications, body weight disorders such as overweight, cachexia and anorexia, and body mass index and leptin related diseases.

As used herein, the term “metabolic syndrome” is a recognized clinical term used to describe a condition comprising combinations of Type II diabetes, impaired glucose tolerance, insulin resistance, hypertension, obesity, increased abdominal girth, hypertriglyceridemia, low HDL, hyperuricaemia, hypercoagulability and/or microalbuminemia.

An SCD-mediated disease or condition also includes various hepatic conditions such as hepatitis, hepatic steatosis, hepatic fibrosis, hepatic cirrhosis, non-alcoholic hepatitis, non-alcoholic steatohepatitis (NASH), alcoholic hepatitis, fatty liver, acute fatty liver, fatty liver of pregnancy, drug-induced hepatitis, erythrohepatic protoporphyria, iron overload disorders, hereditary hemochromatosis, hepatoma and conditions related thereto.

Various skin and mucosal tissue disorders fall within the scope of an SCD-mediated disease or condition including, but not limited to, eczema, acne, psoriasis, keloid scar formation or prevention, diseases related to production or secretions from mucous membranes, such as monounsaturated fatty acids, wax esters, and the like. Inflammation, sinusitis, asthma, pancreatitis, osteoarthritis, rheumatoid arthritis, cystic fibrosis, and pre-menstrual syndrome may also be considered SCD-mediated diseases or conditions as may diseases or conditions which is, or is related to cancer, neoplasia, malignancy, metastases, tumors (benign or malignant), carcinogenesis, hepatomas and the like. SCD-mediated diseases or conditions also include diseases or conditions which are, or are related to, neurological diseases, psychiatric disorders, multiple sclerosis, eye diseases, and immune disorders. An SCD-mediated disease or condition also includes a disease or condition which is, or is related to, viral diseases or infections.

An SCD-mediated disease or condition also includes a condition where increasing lean body mass or lean muscle mass is desired, such as is desirable in enhancing performance through muscle building. Myopathies and lipid myopathies such as carnitine palmitoyltransferase deficiency (CPT I or CPT II) are also included herein. Such treatments are useful in humans and in animal husbandry, including for administration to bovine, porcine or avian domestic animals or any other animal to reduce triglyceride production and/or provide leaner meat products and/or healthier animals.

### Testing

The identification of compounds of the invention as SCD inhibitors was readily accomplished using the SCD enzyme and microsomal assay procedure described in Talamo and Bloch (1969) Analytical Biochemistry 29:300-304. When tested in this assay, compounds of the invention had less than 50% remaining SCD activity at 10 μM concentration of the test compound, preferably less than 40% remaining SCD activity at 10 μM concentration of the test compound, more preferably less than 30% remaining SCD activity at 10 μM concentration of the test compound, and even more preferably less than 20% remaining SCD activity at 10 μM concentration of the test compound, thereby demonstrating that the compounds of the invention are potent inhibitors of SCD activity.

Other methods of testing the compounds disclosed herein are also readily available to those skilled in the art. Thus, in addition, testing of the compounds may be accomplished in vivo. In one such embodiment, testing of the compounds is accomplished by administering the compound to an animal afflicted with a plasma or tissue, fatty acid or triglyceride (TG) related disorder or very low density lipoprotein (VLDL)-related disorder and subsequently detecting a change in plasma or tissue fatty acid composition or triglyceride level in said animal thereby identifying a therapeutic agent useful in treating a plasma or tissue, fatty acid or triglyceride (TG) related disorder or very low density lipoprotein (VLDL)-related disorder. In such embodiment, the animal may be a human, such as a human patient afflicted with such a disorder and in need of treatment of said disorder.

In specific embodiments of such in vivo processes, said change in SCD activity in said animal is a decrease in activity, preferably wherein said SCD modulating agent does not substantially directly inhibit the biological activity of a Δ5 desaturase, Δ6 desaturase, or fatty acid synthetase or other lipogenic enzymes.

The model systems useful for compound evaluation may include, but not limited to, the use of liver microsomes, such as from mice or rats that have been maintained on a high carbohydrate or high-fate diet, or from human donors, including persons suffering from obesity. Immortalized cell lines, such as HepG2 (from human liver), MCF-7 (from human breast cancer) and 3T3-L1 (from mouse adipocytes) may also be used. Primary cell lines, such as primary hepatocytes and adipocytes, are also useful in testing the compounds of the invention. Where whole animals are used, mice or rats used as a source of primary hepatocyte cells may also be used wherein the mice or rats have been maintained on a high carbohydrate or other SCD inducing diet to increase SCD activity in microsomes and/or to elevate plasma triglyceride levels or Δ9 fatty acid desaturation indexes (i.e., the 18:1/18:0 ratio); alternatively mice on a normal diet or mice with normal triglyceride levels may be used. Mouse models employing transgenic mice designed for hypertriglyceridemia are also available. Rabbits, hamsters, and monkeys are also useful as animal models, especially those with diabetic and obesity.

Another suitable method for determining the in vivo efficacy of the compounds of the invention is to indirectly measure their impact on inhibition of SCD enzyme by measuring changes in fatty acid composition. These include absolute or relative reductions in SCD product fatty acids such as 16:1 n-7, 18:1 n-7 or 18:1 n-9. As well fatty acid composition data may also be used to determine a subject's Δ9 Desaturation Index after administration of the compound. “Desaturation Index(s)” as employed in this specification means the ratio of the product over the substrate for the SCD enzyme as measured from a given tissue sample. This may be calculated using different equations, such as 18:1n-9/18:0; 16:1n-7/16:0; and/or (16:1n-7+18:1n-7)/16:0. Desaturation Index(s) may be measured in plasma or tissues as well as specific lipid classes containing fatty acids such as triglycerides and phospholipids.

### Administration

The compounds of Formula I may be administered in either single or multiple doses by any of the accepted modes of administration of agents having similar utilities, for example as described in those patents and patent applications incorporated by reference, including buccal, intranasal, intra-arterial injection, intravenously, intraperitoneally, parenterally, intramuscularly, subcutaneously, orally, or as an inhalant.

Oral administration is the preferred route for administration of the compounds of Formula I. Administration may be via capsule or enteric coated tablets, or the like. In making the pharmaceutical compositions that include at least one compound of Formula I, the active ingredient is usually diluted by an excipient and/or enclosed within such a carrier that can be in the form of a capsule, sachet, paper or other container. When the excipient serves as a diluent, in can be a solid, semi-solid, or liquid material (as above), which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, sterile injectable solutions, and sterile packaged powders.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, cyclodextrins, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents.

The compositions of the invention can be formulated so as to provide quick, sustained or delayed release of the active ingredient after administration to the patient by employing procedures known in the art. Controlled release drug delivery systems for oral administration include osmotic pump systems and dissolutional systems containing polymer-coated reservoirs or drug-polymer matrix formulations. Examples of controlled release systems are given in U.S. Pat. Nos. 3,845,770; 4,326,525; 4,902,514; and 5,616,345.

Another formulation for use in the methods of the present invention employs transdermal delivery devices (“patches”). Such transdermal patches may be used to provide continuous or discontinuous infusion of the compounds of the present invention in controlled amounts. The construction and use of transdermal patches for the delivery of pharmaceutical agents is well known in the art. See, e.g., U.S. Pat. Nos. 5,023,252, 4,992,445 and 5,001,139. Such patches may be constructed for continuous, pulsatile, or on demand delivery of pharmaceutical agents.

SCD inhibitors such as the compounds of Formula I are effective over a wide dosage range and is generally administered in a pharmaceutically effective amount. Typically, for oral administration, each dosage unit contains from 1 mg to 2 g of an SCD inhibitor, more commonly from 1 to 700 mg, and for parenteral administration, from 1 to 700 mg of a stearoyl-CoA desaturase inhibitor, more commonly about 2 to 200 mg. It will be understood, however, that the amount of the SCD inhibitor actually administered will be determined by a physician, in the light of the relevant circumstances, including the condition to be treated, the chosen route of administration, the actual compound administered and its relative activity, the age, weight, and response of the individual patient, the severity of the patient's symptoms, and the like.

For preparing solid compositions such as tablets, the principal active ingredient is mixed with a pharmaceutical excipient to form a solid preformulation composition containing a homogeneous mixture of a compound of the present invention. When referring to these preformulation compositions as homogeneous, it is meant that the active ingredient is dispersed evenly throughout the composition so that the composition may be readily subdivided into equally effective unit dosage forms such as tablets, pills and capsules.

The tablets or pills of the present invention may be coated or otherwise compounded to provide a dosage form affording the advantage of prolonged action, or to protect from the acid conditions of the stomach. For example, the tablet or pill can comprise an inner dosage and an outer dosage component, the latter being in the form of an envelope over the former. The two components can be separated by an enteric layer that serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. Preferably the compositions are administered by the oral or nasal respiratory route for local or systemic effect. Compositions in preferably pharmaceutically acceptable solvents may be nebulized by use of inert gases. Nebulized solutions may be inhaled directly from the nebulizing device or the nebulizing device may be attached to a face mask tent, or intermittent positive pressure breathing machine. Solution, suspension, or powder compositions may be administered, e.g. orally or nasally, from devices that deliver the formulation in an appropriate manner.

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLE 1

### Preparation of a Compound of Formula (3)

A. Preparation of a Compound of Formula (3) in which R² and R³ are Chloro, R⁴ is Hydrogen, and Y is Methylene

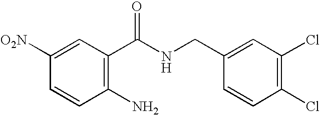

To a mixture of 0.5 g (2.7 mmol, 1.0 eq.) of 5-nitroanthranilic acid, 0.5 g (3.3 mmol, 1.2 eq.) of HOBt monohydrate and 0.63 g (3.3 mol, 1.2 eq.) of EDC in 30 mL of CH₂Cl₂ was added 0.4 mL (3.0 mmol, 1.1 eq.) 3,4-dichlorobezylamine and the mixture stirred at room temperature for 2 hours. The mixture was diluted with 40 mL of sat. NH₄Cl solution. The organic phase was dried (NaSO₄) and the solvent removed in vacuo. The residue was dried under high vacuum to provide N-(3,4-dichlorobenzyl)-2-amino-5-nitrobenzamide.

### B. Preparation of Compounds of Formula (3) Varying R², R³, R⁴, and Y

Similarly, following the procedure of Example 1A above, but optionally substituting 4-nitroanthranilic acid for 5-nitroanthranilic acid and/or optionally substituting other amines of formula (2) for 4-dichlorobezylamine, the following compound of formula (3) was prepared:
N-(3,4-dichlorobenzyl)-2-amino-4-nitrobenzamide;
N-(3,4-difluorobenzyl)-2-amino-4-nitrobenzamide;
N-(3-chlorobenzyl)-2-amino-4-nitrobenzamide;
N-(4-chlorobenzyl)-2-amino-5-nitrobenzamide;
N-(4-chlorobenzyl)-2-amino-4-nitrobenzamide;
N-(3-fluororobenzyl)-2-amino-4-nitrobenzamide;
N-(4-fluorobenzyl)-2-amino-5-nitrobenzamide;
N-(3-trifluoromethylbenzyl)-2-amino-5-nitrobenzamide;
N-(4-trifluormethylbenzyl)-2-amino-5-nitrobenzamide;
N-(4-bromobenzyl)-2-amino-5-nitrobenzamide;
N-(4-methoxybenzyl)-2-amino-5-nitrobenzamide;
N-(3-chloro-4-fluorobenzyl)-2-amino-5-nitrobenzamide;
N-(3-fluoro-4-chlorobenzyl)-2-amino-5-nitrobenzamide;
(2-amino-5-nitrophenyl)-N-[(4-benzo[b]thiophen-5-ylphenyl)methyl]carboxamide;
(2-amino-5-nitrophenyl)-N-[(4-benzo[b]thiophen-6-ylphenyl)methyl]carboxamide; and
(2-amino-5-nitrophenyl)-N-[2-(3,4-dichlorophenyl)ethyl]carboxamide.

### C. Preparation of Compounds of Formula (3) Varying R², R³, R⁴, and Y

Similarly, following the procedure of Example 1A above, but optionally substituting 4-nitroanthranilic acid for 5-nitroanthranilic acid and/or optionally substituting other amines of formula (2) for 4-dichlorobezylamine, other compounds of formula (3) are prepared.

### EXAMPLE 2

### Preparation of a Compound of Formula (4)

A. Preparation of a Compound of Formula (4) in which R² and R³ are Chloro, R⁴, R⁵, R⁶, and R⁷ are Hydrogen, and Y is Methylene

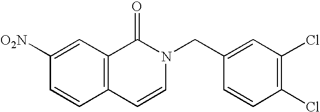

To a suspension of 0.5 g (1.47 mmole, 1.0 eq.) of N-(3,4-dichlorobenzyl)-2-amino-5-nitrobenzamide in 10 mL of trimethylorthoformate in a Biotage microwave vial we added 0.05 g (0.23 mmol, 0.15 eq.) of (D)-10-camphorsulfonic acid. The mixture was subjected to microwave irradiation, maintaining an internal reaction temperature of 180° C. for 30 minutes. The solvent was removed in vacuo to provide crude 3-(3,4-dichlorobenzyl)-6-nitroquinazolin-4-(3H)-one as a white solid. m/z (ESI); found 350.2 [M+H]⁺.

### B. Preparation of Compounds of Formula (4) Varying R², R³, R⁴, R⁵, R⁶, R⁷, and Y

Similarly, following the procedure of Example 2A above, but optionally substituting other compounds of formula (3) for N-(3,4-dichlorobenzyl)-2-amino-5-nitrobenzamide, and optionally substituting triethylorthoformate for trimethylorthoformate, the following compounds of formula (4) were prepared:
3-(3,4-dichlorobenzyl)-7-nitroquinazolin-4-(3H)-one;
3-[(3,4-dichlorophenyl)methyl]-2-methyl-6-nitro-3-hydroquinazolin-4-one
3-(3,4-difluorobenzyl)-7-nitroquinazolin-4-(3H)-one;
3-(3-chlorobenzyl)-7-nitroquinazolin-4-(3H)-one;
3-(4-chlorobenzyl)-6-nitroquinazolin-4-(3H)-one;
3-(4-chlorobenzyl)-7-nitroquinazolin-4-(3H)-one;
3-(3-fluororobenzyl)-7-nitroquinazolin-4-(3H)-one;
3-(4-fluorobenzyl)-6-nitroquinazolin-4-(3H)-one;
3-(3-trifluoromethylbenzyl)-6-nitroquinazolin-4-(3H)-one;
3-(4-trifluormethylbenzyl)-6-nitroquinazolin-4-(3H)-one;
3-(4-bromobenzyl)-6-nitroquinazolin-4-(3H)-one;
3-(4-methoxybenzyl)-6-nitroquinazolin-4-(3H)-one;
3-(3-chloro-4-fluorobenzyl)-6-nitroquinazolin-4-(3H)-one;
3-(3-fluoro-4-chlorobenzyl)-6-nitroquinazolin-4-(3H)-one;
3-[(4-benzo[b]thiophen-6-ylphenyl)methyl]-5-nitro-3-hydroquinazolin-4-one;
3-[(4-benzo[b]thiophen-6-ylphenyl)methyl]-6-nitro-3-hydroquinazolin-4-one; and
6-nitro-3-(2-phenylethyl)-3-hydroquinazolin-4-one.

### C. Preparation of Compounds of Formula (4) Varying R², R³, R⁴, R⁵, R⁶, R⁷, and Y

Similarly, following the procedure of Example 2A above, but optionally substituting other compounds of formula (3) for N-(3,4-dichlorobenzyl)-2-amino-5-nitrobenzamide, and optionally substituting triethylorthoformate for trimethylorthoformate, other compounds of formula (4) are prepared.

### EXAMPLE 3

### Preparation of a Compound of Formula (5)

A. Preparation of a Compound of Formula (5) in which R² and R³ are Chloro, R⁴, R⁵, R⁶, and R⁷ are Hydrogen, and Y is Methylene

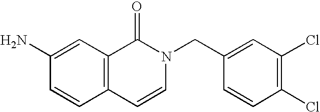

To a solution of 1.47 mmol of crude 3-(3,4-dichlorobenzyl)-6-nitroquinazolin-4-(3H)-one in 30 mL of p-dioxane was added a solution of 2.0 g (tech. grad, 85%, 9.8 mmol, 6.7 eq.) of sodium bisulfite and 1.0 g (9.5 mmol, 6.7 eq.) of sodium carbonate in 20 mL of water. The mixture was stirred at room temperature for 16 hours. The mixture was then diluted with 60 mL of saturated brine. The organic phase was dried (Na₂SO₄) and the solvent removed in vacuo. The product was isolated by flash chromatorgraphy (12 g flash silica column, eluting with linear gradient of 30-100% EtOAc/hexanes over 15 minutes) to provide the compound of formula (5), 3-(3,4-dichlorobenzyl)-6-aminoquinazoline-4-(3H)-one as a brown solid.

δ_{H} (d₆ DMSO, 300 MHz) 5.02 (s, 2H), 6.98 (dd, 1H), 7.82 (d, 1H), 7.21 (dd, 1H), 7.50 (d, 1H), 7.55 (d, 1H), 8.14 (s, 1H); m/z (ESI); found 320.1 [M+H]⁺.

B. Alternative Preparation of Compounds of Formula (5) in which R² and R³ are Chloro, R⁴, R⁵, R⁶, and R⁷ Hydrogen, and Y is Methylene

To a solution of 6-nitro-3-(3,4-dichlorobenzyl)quinazolin-4(3H)-one (1.25 g, 3.58 mmol) and hydrazine hydrate (1.8 g, 36 mmol) in methanol (100 ml) at 50° C. Ni Raney (1 g) was added portion wise. After gas evolution has ceased, stirring was continued for additional 10 min at 50° C., and reaction mixture was quickly filtered through a 1-2 cm plug of silica gel. The resulting solution was concentrated, the resulting crystalline solid washed with ethyl acetate and dried to give the compound of formula (5), 3-(3,4-dichlorobenzyl)-6-aminoquinazoline-4-(3H)-one.

### C. Preparation of Compounds of Formula (5) Varying R², R³, R⁴, R⁵, R⁶, R⁷, and Y

Similarly, following the procedure of Example 3A or 3B, but optionally substituting other compounds of formula (4) for 3-(3,4-dichlorobenzyl)-6-nitroquinazolin-4-(3H)-one, the following compounds of formula (5) were prepared:
3-(3,4-dichlorobenzyl)-7-aminoquinazolin-4-(3H)-one;
3-[(3,4-dichlorophenyl)methyl]-2-methyl-6-amino-3-hydroquinazolin-4-one
3-(3,4-difluorobenzyl)-7-aminoquinazolin-4-(3H)-one;
3-(3-chlorobenzyl)-7-aminoquinazolin-4-(3H)-one;
3-(4-chlorobenzyl)-6-aminoquinazolin-4-(3H)-one;
3-(4-chlorobenzyl)-7-aminoquinazolin-4-(3H)-one;
3-(3-fluororobenzyl)-7-aminoquinazolin-4-(3H)-one;
3-(4-fluorobenzyl)-6-aminoquinazolin-4-(3H)-one;
3-(3-trifluoromethylbenzyl)-6-aminoquinazolin-4-(3H)-one;
3-(4-trifluormethylbenzyl)-6-aminoquinazolin-4-(3H)-one;
3-(4-bromobenzyl)-6-aminoquinazolin-4-(3H)-one;
3-(4-methoxybenzyl)-6-aminoquinazolin-4-(3H)-one;
3-(3-chloro-4-fluorobenzyl)-6-aminoquinazolin-4-(3H)-one;
3-(3-fluoro-4-chlorobenzyl)-6-aminoquinazolin-4-(3H)-one;
3-[(4-benzo[b]thiophen-6-ylphenyl)methyl]-5-amino-3-hydroquinazolin-4-one;
3-[(4-benzo[b]thiophen-6-ylphenyl)methyl]-6-amino-3-hydroquinazolin-4-one; and
6-amino-3-(2-phenylethyl)-3-hydroquinazolin-4-one.

### C. Preparation of Compounds of Formula (5) Varying R², R³, R⁴, R⁵, R⁶, R⁷, and Y

Similarly, following the procedure of Example 3A or 3B, but optionally substituting other compounds of formula (4) for 3-(3,4-dichlorobenzyl)-6-nitroquinazolin-4-(3H)-one, and optionally substituting triethylorthoformate for trimethylorthoformate, other compounds of formula (5) are prepared.

### EXAMPLE 4

### Preparation of a Compound of Formula I

A. Preparation of a Compound of Formula I in which R¹ is Benzyl, R² and R³ are Chloro, R⁴, R⁵, and R⁷ are Hydrogen, Y is Methylene, and X is Oxygen

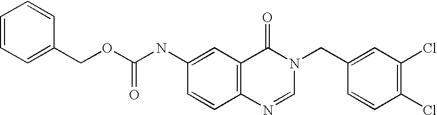

To a solution of 15 mg (47 μmol, 1.0 eq.) of 3-(3,4-dichlorobenzyl)-6-aminoquinazolin-4-(3H)-one in 0.75 mL of p-dioxane was added a solution of 20 mg (0.19 mmol, 4.0 eq.) of sodium carbonate in 0.25 mL of water. A portion of 20 μL (0.12 mmol, 2.5 eq.) of benzyl chloroformate was then added and the mixture stirred at room temperature for 1 hour. The mixture was then diluted with 5 mL of CH₂Cl₂ and 1 mL of sat. Na₂CO₃ solution. The layers were separated and the organic phase dried (Na₂SO₄). The solvent was removed in vacuo and the product isolated by revese-phase chromatography to provide the compound of Formula 1,3-(3,4-dichlorobenzyl)-4-oxo-3,4-dihdroquinazolin-6-yl carbamate as a white solid.

δ_{H}(CDCl₃, 300 MHz) 5.04 (s, 2H), 5.22 (s, 2H), 7.14 (dd, 1H), 7.37 (m, 7H), 7.58 (bs, 1H), 7.71 (m, 1H), 7.99 (s, 1H), 8.24 (m, 2H); m/z (ESI); found 454.2 [M+H]⁺.

### B. Preparation of Compounds of Formula I Varying R¹, R², R³, R⁴, R⁵, R⁶, R⁷, Y, and X

Similarly, following the procedure of Example 4A above, but optionally substituting other compounds of formula (6) for benzyl chloroformate and/or replacing other compounds of formula (5) for 3-(3,4-dichlorobenzyl)-6-aminoquinazolin-4-(3H)-one, the following compounds of Formula I were prepared:
N-{3-[(3,4-dichlorophenyl)methyl]-4-oxo(3-hydroquinazolin-6-yl)}-3-phenylpropanamide;
N-{3-[(3,4-dichlorophenyl)methyl]-4-oxo-3-hydroquinazolin-6-yl}hexanamide;
N-{3-[(3,4-dichlorophenyl)methyl]-2-methyl-4-oxo(3-hydroquinazolin-6-yl)}(phenylmethoxy)carboxamide;
N-{3-[(3,4-dichlorophenyl)methyl]-4-oxo(3-hydroquinazolin-6-yl)}(2-methylpropoxy)carboxamide;
N-{3-[(4-chlorophenyl)methyl]-4-oxo(3-hydroquinazolin-6-yl)}(phenylmethoxy)carboxamide;
N-{3-[(3,4-dichlorophenyl)methyl]-4-oxo(3-hydroquinazolin-6-yl)}(methylethoxy)carboxamide;
N-{3-[(3,4-dichlorophenyl)methyl]-4-oxo(3-hydroquinazolin-6-yl)}prop-2-enyloxycarboxamide;
N-{3-[(3,4-dichlorophenyl)methyl]-4-oxo(3-hydroquinazolin-6-yl)}(4-methylphenoxy)carboxamide;
N-{3-[(3,4-dichlorophenyl)methyl]-4-oxo(3-hydroquinazolin-6-yl)}(4-fluorophenoxy)carboxamide;
N-{3-[(3,4-dichlorophenyl)methyl]-4-oxo(3-hydroquinazolin-6-yl)}methoxycarboxamide;
N-{3-[(4-chlorophenyl)methyl]-4-oxo(3-hydroquinazolin-6-yl)}prop-2-enyloxycarboxamide;
N-{3-[(3,4-dichlorophenyl)methyl]-4-oxo(3-hydroquinazolin-6-yl)}-2-phenoxyacetamide;
N-{3-[2-(4-chlorophenyl)ethyl]-4-oxo(3-hydroquinazolin-6-yl)}(phenylmethoxy)carboxamide;
N-{3-[(3,4-dichlorophenyl)methyl]-4-oxo(3-hydroquinazolin-6-yl)}-2-(4-methoxyphenoxy)acetamide;
N-{3-[(3,4-dichlorophenyl)methyl]-4-oxo(3-hydroquinazolin-6-yl)}-2-(4-fluorophenoxy)acetamide;
2-(acetylamino)-N-{3-[(3,4-dichlorophenyl)methyl]-4-oxo(3-hydroquinazolin-6-yl)}acetamide;
N-{3-[(3,4-dichlorophenyl)methyl]-4-oxo(3-hydroquinazolin-6-yl)}-2-(3,5-dimethylphenoxy)acetamide;
N-{3-[(3,4-dichlorophenyl)methyl]-4-oxo(3-hydroquinazolin-6-yl)}-2-(2,6-dimethylphenoxy)acetamide;
N-{3-[(3,4-dichlorophenyl)methyl]-4-oxo(3-hydroquinazolin-6-yl)}-2-(4-methylphenoxy)acetamide;
N-{3-[(3,4-dichlorophenyl)methyl]-4-oxo(3-hydroquinazolin-6-yl)}(3-pyridylmethoxy)carboxamide;
N-{3-[(4-fluorophenyl)methyl]-4-oxo(3-hydroquinazolin-6-yl)}(phenylmethoxy)carboxamide;
N-{3-[(3,4-dichlorophenyl)methyl]-4-oxo(3-hydroquinazolin-6-yl)}-2-(2-methoxyphenoxy)acetamide;
N-{3-[(3,4-dichlorophenyl)methyl]-4-oxo(3-hydroquinazolin-6-yl)}-2-(2-fluorophenoxy)acetamide;
N-{3-[(3,4-dichlorophenyl)methyl]-4-oxo(3-hydroquinazolin-6-yl)}-2-(3-fluorophenoxy)acetamide;
N-{3-[(3,4-dichlorophenyl)methyl]-4-oxo(3-hydroquinazolin-6-yl)}-2-(2,4-difluorophenoxy)acetamide;
N-{3-[(3,4-dichlorophenyl)methyl]-4-oxo(3-hydroquinazolin-6-yl)}-2-(2-chlorophenoxy)acetamide;
N-{3-[(3,4-dichlorophenyl)methyl]-4-oxo(3-hydroquinazolin-6-yl)}-2-(2,3-dichlorophenoxy)acetamide;
N-{3-[(3,4-dichlorophenyl)methyl]-4-oxo(3-hydroquinazolin-6-yl)}-2-(2-nitrophenoxy)acetamide;
N-{3-[(3,4-dichlorophenyl)methyl]-4-oxo(3-hydroquinazolin-6-yl)}-2-(2-naphthyloxy)acetamide;
N-{3-[(3,4-dichlorophenyl)methyl]-4-oxo(3-hydroquinazolin-6-yl)}-3-phenoxypropanamide;
N-{3-[(3,4-dichlorophenyl)methyl]-4-oxo(3-hydroquinazolin-6-yl)}-2-(2-chlorophenoxy)propanamide;
2-benzo[c]1,2,5-thiadiazol-4-yloxy-N-{3-[(3,4-dichlorophenyl)methyl]-4-oxo(3-hydroquinazolin-6-yl)}acetamide;
N-{3-[(3,4-dichlorophenyl)methyl]-4-oxo(3-hydroquinazolin-6-yl)}-2-(3-pyridyloxy)acetamide;
N-{3-[(4-chlorophenyl)methyl]-4-oxo(3-hydroquinazolin-6-yl)}-2-(4-pyridylthio)acetamide;
N-{3-[(4-bromophenyl)methyl]-4-oxo(3-hydroquinazolin-6-yl)}-2-phenoxyacetamide;
N-{3-[(4-chloro-3-fluorophenyl)methyl]-4-oxo(3-hydroquinazolin-6-yl)}-2-phenoxyacetamide;
N-{3-[(3-chloro-4-fluorophenyl)methyl]-4-oxo(3-hydroquinazolin-6-yl)}-2-phenoxyacetamide;
N-[3-(benzo[b]thiophen-6-ylmethyl)-4-oxo(3-hydroquinazolin-6-yl)]-2-phenoxyacetamide;
N-{3-[(3,4-dichlorophenyl)methyl]-4-oxo(3-hydroquinazolin-6-yl)}-4-oxo-4-phenylbutanamide;
N-{3-[(4-chlorophenyl)methyl]-4-oxo(3-hydroquinazolin-6-yl)}-2-(3-fluorophenoxy)acetamide;
N-{3-[(4-chlorophenyl)methyl]-4-oxo(3-hydroquinazolin-7-yl)}-2-phenoxyacetamide;
N-[3-(benzo[b]thiophen-5-ylmethyl)-4-oxo(3-hydroquinazolin-6-yl)](phenylmethoxy)carboxamide;
N-{3-[(3-chloro-4-fluorophenyl)methyl]-4-oxo(3-hydroquinazolin-6-yl)}(phenylmethoxy)carboxamide;
(N-{3-[(3,4-dichlorophenyl)methyl]-4-oxo-3-hydroquinazolin-6-yl}carbamoyl)methyl acetate;
N-{3-[(4-chlorophenyl)methyl]-4-oxo(3-hydroquinazolin-7-yl)}(phenylmethoxy)carboxamide;
N-{3-[(4-chlorophenyl)methyl]-4-oxo(3-hydroquinazolin-7-yl)}-2-(3-pyridyloxy)acetamide;
N-{3-[(3,4-dichlorophenyl)methyl]-4-oxo(3-hydroquinazolin-7-yl)}-2-(2-methoxyphenoxy)acetamide;
N-{3-[(3,4-dichlorophenyl)methyl]-4-oxo(3-hydroquinazolin-7-yl)}-2-(3-pyridyloxy)acetamide;
N-{3-[(3,4-dichlorophenyl)methyl]-4-oxo(3-hydroquinazolin-7-yl)}(phenylmethoxy)carboxamide;
N-{3-[(3,4-dichlorophenyl)methyl]-4-oxo(3-hydroquinazolin-7-yl)}-2-phenoxyacetamide;
N-{3-[(3,4-dichlorophenyl)methyl]-4-oxo(3-hydroquinazolin-6-yl)}-2-(4-pyridylthio)acetamide;
N-{3-[(3,4-dichlorophenyl)methyl]-4-oxo(3-hydroquinazolin-6-yl)}-2-pyrimidin-2-ylthioacetamide;
N-{3-[(3,4-dichlorophenyl)methyl]-4-oxo(3-hydroquinazolin-6-yl)}-2-(4-chlorophenylthio)acetamide;
N-{3-[(4-chlorophenyl)methyl]-4-oxo(3-hydroquinazolin-6-yl)}[5-(imidazolylmethyl)(2-furyl)]carboxamide;
N-{3-[(3-chlorophenyl)methyl]-4-oxo(3-hydroquinazolin-7-yl)}(phenylmethoxy)carboxamide;
N-{3-[(3-chlorophenyl)methyl]-4-oxo(3-hydroquinazolin-7-yl)}(3-pyridylmethoxy)carboxamide;
N-{3-[(3,4-dichlorophenyl)methyl]-4-oxo(3-hydroquinazolin-6-yl)}-2-(phenylmethylthio)acetamide;
N-{3-[(3,4-dichlorophenyl)methyl]-4-oxo(3-hydroquinazolin-7-yl)}[(2-methoxyphenyl)methoxy]carboxamide;
N-{3-[(3,4-dichlorophenyl)methyl]-4-oxo(3-hydroquinazolin-6-yl)}-2-hydroxyacetamide;
N-{3-[(3,4-dichlorophenyl)methyl]-4-oxo(3-hydroquinazolin-6-yl)}(2-furylmethoxy)carboxamide;
(N-{3-[(3,4-dichlorophenyl)methyl]-4-oxo-3-hydroquinazolin-7-yl}carbamoyl)methyl acetate;
(N-{3-[(3-chlorophenyl)methyl]-4-oxo-3-hydroquinazolin-7-yl}carbamoyl)methyl acetate;
(N-{3-[(4-chlorophenyl)methyl]-4-oxo-3-hydroquinazolin-7-yl}carbamoyl)methyl acetate;
N-{3-[(3,4-dichlorophenyl)methyl]-4-oxo(3-hydroquinazolin-7-yl)}(2-methoxyethoxy)carboxamide;
N-{3-[(3-chlorophenyl)methyl]-4-oxo(3-hydroquinazolin-7-yl)}-2-hydroxyacetamide;
N-{3-[(3-fluorophenyl)methyl]-4-oxo(3-hydroquinazolin-7-yl)}(phenylmethoxy)carboxamide;
N-{3-[(3,4-dichlorophenyl)methyl]-4-oxo(3-hydroquinazolin-6-yl)}(4-chlorophenoxy)carboxamide;
N-{3-[(3,4-dichlorophenyl)methyl]-4-oxo(3-hydroquinazolin-6-yl)}(2-phenylethoxy)carboxamide;
(N-{3-[(3-fluorophenyl)methyl]-4-oxo-3-hydroquinazolin-7-yl}carbamoyl)methyl acetate;
N-{3-[(3-chloro-4-fluorophenyl)methyl]-4-oxo(3-hydroquinazolin-7-yl)}(phenylmethoxy)carboxamide;
N-{3-[(3-chloro-4-fluorophenyl)methyl]-4-oxo(3-hydroquinazolin-7-yl)}(3-pyridylmethoxy)carboxamide;
(N-{3-[(3-chloro-4-fluorophenyl)methyl]-4-oxo-3-hydroquinazolin-7-yl}carbamoyl)methyl acetate;
N-{3-[(3-fluorophenyl)methyl]-4-oxo(3-hydroquinazolin-7-yl)}(3-pyridylmethoxy)carboxamide;
N-{3-[(4-chloro-3-fluorophenyl)methyl]-4-oxo(3-hydroquinazolin-7-yl)}(3-pyridylmethoxy)carboxamide;
(N-{3-[(4-chloro-3-fluorophenyl)methyl]-4-oxo-3-hydroquinazolin-7-yl}carbamoyl)methyl acetate;
N-{3-[(3,4-dichlorophenyl)methyl]-4-oxo(3-hydroquinazolin-6-yl)}(4-pyridylmethoxy)carboxamide;
N-{3-[(3,4-dichlorophenyl)methyl]-4-oxo(3-hydroquinazolin-6-yl)}[(3-cyanophenyl)methoxy]carboxamide;
N-{3-[(4-chlorophenyl)methyl]-4-oxo(3-hydroquinazolin-7-yl)}-2-hydroxyacetamide;
N-{3-[(3,4-dichlorophenyl)methyl]-4-oxo(3-hydroquinazolin-7-yl)}-2-hydroxyacetamide;
N-{3-[(4-chloro-3-fluorophenyl)methyl]-4-oxo(3-hydroquinazolin-7-yl)}(phenylmethoxy)carboxamide;
N-{3-[(3,4-dichlorophenyl)methyl]-4-oxo(3-hydroquinazolin-6-yl)}{2-[methylbenzylamino]ethoxy}carboxamide;
N-{3-[(3,4-dichlorophenyl)methyl]-4-oxo(3-hydroquinazolin-6-yl)}(cyclopropylmethoxy)carboxamide;
(N-{3-[(3,4-dichlorophenyl)methyl]-4-oxo-3-hydroquinazolin-6-yl}carbamoyl)methyl 2-(dimethylamino)acetate;
N-{3-[(3-chloro-4-fluorophenyl)methyl]-4-oxo(3-hydroquinazolin-7-yl)}-2-hydroxyacetamide;
ethyl 2-(N-{3-[(3,4-dichlorophenyl)methyl]-4-oxo-3-hydroquinazolin-6-yl}carbamoyloxy)acetate;
N-{3-[(3,4-dichlorophenyl)methyl]-4-oxo(3-hydroquinazolin-6-yl)}(2-pyridylmethoxy)carboxamide;
(N-{3-[(3,4-dichlorophenyl)methyl]-4-oxo-3-hydroquinazolin-7-yl}carbamoyl)methyl 2-(dimethylamino)acetate;
ethyl 2-(N-{3-[(3,4-dichlorophenyl)methyl]-4-oxo-3-hydroquinazolin-7-yl}carbamoyloxy)acetate;
N-{3-[(3,4-dichlorophenyl)methyl]-4-oxo(3-hydroquinazolin-6-yl)}[2-(2-methoxyethoxy)ethoxy]carboxamide;
[N-(4-oxo-3-{[3-(trifluoromethyl)phenyl]methyl}-3-hydroquinazolin-6-yl)carbamoyl]methyl acetate;
2-hydroxy-N-(4-oxo-3-{[3-(trifluoromethyl)phenyl]methyl}(3-hydroquinazolin-6-yl))acetamide;
[N-(4-oxo-3-{[4-(trifluoromethyl)phenyl]methyl}-3-hydroquinazolin-6-yl)carbamoyl]methyl acetate;
2-hydroxy-N-(4-oxo-3-{[4-(trifluoromethyl)phenyl]methyl}(3-hydroquinazolin-6-yl))acetamide;
(N-{3-[(4-chloro-3-fluorophenyl)methyl]-4-oxo-3-hydroquinazolin-6-yl}carbamoyl)methyl acetate;
N-{3-[(4-chloro-3-fluorophenyl)methyl]-4-oxo(3-hydroquinazolin-6-yl)}-2-hydroxyacetamide;
(N-{3-[(4-methoxyphenyl)methyl]-4-oxo-3-hydroquinazolin-6-yl}carbamoyl)methyl acetate;
2-hydroxy-N-{3-[(4-methoxyphenyl)methyl]-4-oxo(3-hydroquinazolin-6-yl)}acetamide;
(N-{3-[(3,4-difluorophenyl)methyl]-4-oxo-3-hydroquinazolin-7-yl}carbamoyl)methyl acetate; and
N-{3-[(3,4-difluorophenyl)methyl]-4-oxo(3-hydroquinazolin-7-yl)}-2-hydroxyacetamide.

### C. Preparation of Compounds of Formula I Varying R¹, R², R³, R⁴, R⁵, R⁶, R⁷, Y and X

Similarly, following the procedure of Example 4A above, but optionally substituting other compounds of formula (6) for benzyl chloroformate and/or replacing other compounds of formula (5) for 3-(3,4-dichlorobenzyl)-6-aminoquinazolin-4-(3H)-one, other compounds of Formula I are prepared.

### EXAMPLE 5

### Preparation of a Compound of Formula I

### Preparation of a Compound of Formula I in which R¹ is Pentyl, R² and R³ are Chloro, R⁴, R⁵, R⁶, and R⁷ are Hydrogen, Y is Methylene, and X is a Covalent Bond

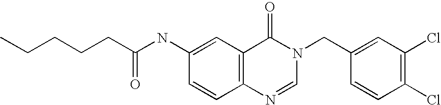

To a solution of 44 mg (0.14 mmol, 1.0 eq.) of 3-(3,4-dichlorobenzyl)-6-aminoquinazolin-4-(3H)-one in 3 mL of p-dioxane was added 47 μL of hexanoyl chloride (6.18, 0.34 mmol, 2.4 eq.) followed by Na₂CO₃ solution in water (50 mg in 0.7 mL). The reaction mixture was stirred at room temperature overnight. After extractive workup the crude reaction mixture was purified on prep-TLC eluting with 3:1 ethyl acetate/hexanes. The desired product, N-(3-(3,4-dichlorobenzyl)-4-oxo-3,4-dihydroquinazolin-6-yl)hexanamide, was obtained as colorless solid.

MS (ESI, m/z): 417.95 [M+H⁺].

¹H NMR (δ, CDCl₃, 400 MHz): 8.28 (dd, 1H); 8.14 (d, 1H); 8.03 (s, 1H); 7.71 (d, 1H); 7.50 (br s, 1H); 7.45 (d, 1H); 7.43 (d, 1H); 7.19 (dd, 1H); 5.13 (s, 2H); 2.41 (t, 2H); 1.75 (quintet, 2H); 1.41-1.33 (m, 4H); 0.92 (t, 3H).

### EXAMPLE 6

### Preparation of a Compound of Formula I

A. Preparation of a Compound of Formula I in which R¹ is Pentyl, R² and R³ are Chloro, R⁴, R⁵, R⁶, and R⁷ are Hydrogen, Y is Methylene, and X is NH

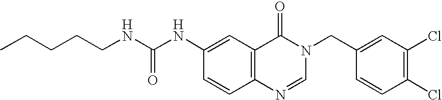

To a solution of 49.6 mg (15.5 mmol, 1.0 eq.) of 3-(3,4-dichlorobenzyl)-6-aminoquinazolin-4-(3H)-one in 3 mL of p-dioxane was added 50 mL of n-pentyl isocyanate (0.38 mmol, 2.5 eq.) followed by Na₂CO₃ solution in water (50 mg in 0.7 mL). The reaction mixture was stirred at room temperature overnight. After extractive workup the crude reaction mixture was purified on prep-TLC eluting with 3% MeOH in CH₂Cl₂. The desired product, 1-(3-(3,4-dichlorobenzyl)-4-oxo-3,4-dihydroquinazolin-6-yl)-3-pentylurea, was obtained as colorless solid.

MS (ESI, m/z): 432.99 [M+H⁺].

¹H NMR (δ, CDCl₃, 400 MHz): 8.14 (dd, 1H); 8.03 (s, 1H); 7.94 (d, 1H); 7.67 (d, 1H); 7.43 (d, 1H); 7.42 (d, 1H); 7.17 (dd, 1H); 7.11 (br s, 1H); 5.12 (s, 2H); 5.06 (br t, 1H); 3.25 (q, 2H); 1.52 (quintet, 2H); 1.35-1.27 (m, 4H); 0.89 (t, 3H).

### B. Preparation of Compounds of Formula I Varying R¹, R², R³, R⁴, R⁵, R⁶, R⁷, Y and X

Similarly, following the procedure of Example 6A above, but optionally substituting other compounds of formula (6′) for n-pentyl isocyanate and/or replacing other compounds of formula (5) for 3-(3,4-dichlorobenzyl)-6-aminoquinazolin-4-(3H)-one, the following compounds of formula (4) were prepared:
N-{3-[(3,4-dichlorophenyl)methyl]-4-oxo(3-hydroquinazolin-6-yl)}[benzylamino]carboxamide; and
N-{3-[(3,4-dichlorophenyl)methyl]-4-oxo(3-hydroquinazolin-6-yl)}[(2-methoxyethyl)amino]carboxamide.

### C. Preparation of Compounds of Formula I Varying R¹, R², R³, R⁴, R⁵, R⁶, R⁷, Y and X

Similarly, following the procedure of Example 6A above, but optionally substituting other compounds of formula (6′) for n-pentyl isocyanate and/or replacing other compounds of formula (5) for 3-(3,4-dichlorobenzyl)-6-aminoquinazolin-4-(3H)-one, other compounds of Formula I are prepared.

### EXAMPLE 7

### Preparation of a Compound of Formula I

### Preparation of a Compound of Formula I in which R¹ is CH₃C(O)OCH₂—, R² and R³ are Chloro, R⁴, R⁵, R⁶, and R⁷ are Hydrogen, Y is Methylene, and X is a Covalent Bond

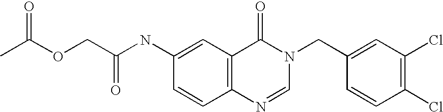

A solution containing acetylglycolic acid (250 mg, 2.11 mmol) and carbonyldiimidazole (411 mg, 2.5 mmol) in methylene chloride (10 ml) was stirred at room temperature for 1 hr, then 6-amino-3-(3,4-dichlorobenzyl)quinazolin-4(3H)-one (200 mg, 0.62 mmol) was added and stirring was continued at 70° C. for 3 h. The reaction mixture was concentrated and subjected to column chromatography (chloroform−ethyl acetate 10:1 to 5:1) to afford the title product, N-(3-(3,4-dichlorobenzyl)-4-oxo-3,4-dihydroquinazolin-6-yl)-2-acetoxyacetamide.

MS (APCI, m/z): 419.94 [M+H⁺].

¹H NMR (δ, CDCl₃, 400 MHz): 10.36 (s, 1H); 8.49 (m, 2H); 8.00 (d, 1H); 7.69 (d, 1H); 7.60 (d, 1H); 7.38 (d, 1H); 5.20 (s, 2H); 4.70 (s, 2H); 2.12 (s, 3H).

### EXAMPLE 8

### Preparation of a Compound of Formula I

### Preparation of a Compound of Formula I in which R¹ is Hydroxymethyl, R² and R³ are Chloro, R⁴, R⁵, R⁶ and R⁷ are Hydrogen, Y is Methylene, and X is a Covalent Bond

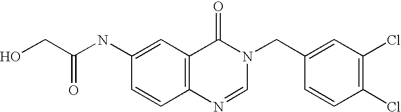

To a solution of 50 mg (0.12 mmol) of N-(3-(3,4-dichlorobenzyl)-4-oxo-3,4-dihydroquinazolin-6-yl)-2-acetoxyacetamide in 2 mL of methanol was added 2M aqueous solution of LiOH (2 mL) and stirred at room temperature overnight. Solvents were removed in vacuo. The residue was redissolved in CH₂Cl₂ and washed consecutively with 0.2 N HCl and water. Organic layer was dried over Na₂SO₄, concentrated and subjected to flash-chromatography (CH₂Cl₂) to provide the title product, N-(3-(3,4-dichlorobenzyl)-4-oxo-3,4-dihydroquinazolin-6-yl)-2-hydroxyacetamide.

MS (ESI, m/z): 377.91 [M+H⁺].

¹H NMR (δ, CDCl₃, 400 MHz): 9.99 (brs, 1H); 8.60 (s, 1H); 8.48 (s, 1H); 8.07 (d, 1H); 7.67 (d, 1H); 7.58 (d, 1H); 7.36 (d, 1H); 5.58 (br s, 1H); 5.17 (s, 2H); 4.05 (s, 2H).

### Preparation of Further Compounds of Formula I

Following the procedures set out in Examples 7 and 8 above, but optionally substituting other starting materials as may be determined by inspection of the final compound, the following compounds of Formula I were prepared:
N-(3-(3,4-difluorobenzyl)-4-oxo-3,4-dihydroquinazolin-6-yl)-2-hydroxyacetamide;
N-(3-(4-chlorobenzyl)-4-oxo-3,4-dihydroquinazolin-6-yl)-2-hydroxyacetamide;
N-(3-(4-chloro-3-(trifluoromethyl)benzyl)-4-oxo-3,4-dihydroquinazolin-6-yl)-2-hydroxyacetamide;
N-(3-(1-(3,4-dichlorophenyl)ethyl)-4-oxo-3,4-dihydroquinazolin-7-yl)-2-hydroxyacetamide;
N-(3-(4-fluoro-3-(trifluoromethyl)benzyl)-4-oxo-3,4-dihydroquinazolin-6-yl)-2-hydroxyacetamide;
N-(3-(3,4-dimethylbenzyl)-4-oxo-3,4-dihydroquinazolin-6-yl)-2-hydroxyacetamide;
N-(3-(1-(3,4-dichlorophenyl)ethyl)-4-oxo-3,4-dihydroquinazolin-6-yl)-2-hydroxyacetamide;
N-(3-(1-(3,4-dichlorophenyl)propyl)-4-oxo-3,4-dihydroquinazolin-6-yl)-2-hydroxyacetamide;
N-(3-benzyl-4-oxo-3,4-dihydroquinazolin-6-yl)-2-hydroxyacetamide;
N-(3-(biphenyl-3-ylmethyl)-4-oxo-3,4-dihydroquinazolin-6-yl)-2-hydroxyacetamide;
(R)—N-(3-(1-(4-chloro-3-(trifluoromethyl)phenyl)ethyl)-4-oxo-3,4-dihydroquinazolin-6-yl)-2-hydroxyacetamide;
(S)—N-(3-(1-(3,4-dichlorophenyl)ethyl)-4-oxo-3,4-dihydroquinazolin-7-yl)-2-hydroxyacetamide;
(R)—N-(3-(1-(3,4-dichlorophenyl)ethyl)-4-oxo-3,4-dihydroquinazolin-7-yl)-2-hydroxyacetamide;
N-(3-(4-chlorophenethyl)-4-oxo-3,4-dihydroquinazolin-6-yl)-2-hydroxyacetamide;
N-(3-(3-chlorophenethyl)-4-oxo-3,4-dihydroquinazolin-6-yl)-2-hydroxyacetamide;
N-(3-(2,4-dichlorophenethyl)-4-oxo-3,4-dihydroquinazolin-6-yl)-2-hydroxyacetamide;
N-(3-(2-(2,5-dichlorophenoxy)ethyl)-4-oxo-3,4-dihydroquinazolin-6-yl)-2-hydroxyacetamide;
N-(3-(3-(2,5-dichlorophenoxy)propyl)-4-oxo-3,4-dihydroquinazolin-6-yl)-2-hydroxyacetamide;
N-(3-(3-(2-fluorophenoxy)propyl)-4-oxo-3,4-dihydroquinazolin-7-yl)-2-hydroxyacetamide;
N-(3-(3-(2,5-dichlorophenoxy)propyl)-4-oxo-3,4-dihydroquinazolin-7-yl)-2-hydroxyacetamide;
N-(3-(4-(2,5-dichlorophenoxy)butyl)-4-oxo-3,4-dihydroquinazolin-6-yl)-2-hydroxyacetamide;
N-(3-(3-(2-chlorophenyl)propyl)-4-oxo-3,4-dihydroquinazolin-6-yl)-2-hydroxyacetamide;
N-(3-(2-(2-chlorophenoxy)ethyl)-4-oxo-3,4-dihydroquinazolin-6-yl)-2-hydroxyacetamide;
N-(3-(3-(2-chlorophenoxy)propyl)-4-oxo-3,4-dihydroquinazolin-6-yl)-2-hydroxyacetamide;
N-(3-(3-((2,5-dichlorophenoxy)methyl)benzyl)-4-oxo-3,4-dihydroquinazolin-6-yl)-2-hydroxyacetamide;
N-(3-(2-(2-chlorophenoxy)ethyl)-4-oxo-3,4-dihydroquinazolin-7-yl)-2-hydroxyacetamide;
N-(3-(5-(2,5-dichlorophenoxy)pentyl)-4-oxo-3,4-dihydroquinazolin-6-yl)-2-hydroxyacetamide;
N-(3-(3-(2,3-dichlorophenoxy)propyl)-4-oxo-3,4-dihydroquinazolin-6-yl)-2-hydroxyacetamide;
N-(3-(3-(2-chloro-5-(trifluoromethyl)phenoxy)propyl)-4-oxo-3,4-dihydroquinazolin-6-yl)-2-hydroxyacetamide;
N-(3-(3-(4-chlorophenoxy)propyl)-4-oxo-3,4-dihydroquinazolin-6-yl)-2-hydroxyacetamide;
N-(3-(2-(2,5-dichlorophenylsulfonamido)ethyl)-4-oxo-3,4-dihydroquinazolin-6-yl)-2-hydroxyacetamide;
N-(3-(3-(2,5-dichlorophenylsulfonamido)propyl)-4-oxo-3,4-dihydroquinazolin-6-yl)-2-hydroxyacetamide;
N-(3-(2-(4-chlorophenoxy)ethyl)-4-oxo-3,4-dihydroquinazolin-6-yl)-2-hydroxyacetamide;
N-(3-(2-(3-chlorophenoxy)ethyl)-4-oxo-3,4-dihydroquinazolin-6-yl)-2-hydroxyacetamide;
N-(3-(3,4-dichlorobenzyl)-4-oxo-3,4-dihydropyrido[2,3-d]pyrimidin-6-yl)-2-hydroxyacetamide;
N-(3-(4-chlorobenzyl)-4-oxo-3,4-dihydropyrido[2,3-d]pyrimidin-6-yl)-2-hydroxyacetamide.

### EXAMPLE 9

### A. Synthesis of Primary Amines of Formula (2A)

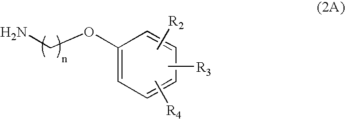

Primary amines of formula (2A) (which are compounds of formula (2), see Reaction Scheme I) can be made by a variety of known synthetic methods including, but not limited, to the following references: Yamazaki, Y. et al, Bioorg. Med. Chem. Lett. 2007, 17, 4689-93; Tan, E. S. et al, J. Med. Chem. 2007, 50, 2787-98; Xie, S.-X. et al, Bioorg. Med. Chem. Lett. 2006, 16, 3886-90; Guizzunti, G. et al, Bioorg. Med. Chem. Lett. 2007, 17, 320-5; Dawson, M. I. et al, J. Med. Chem. 2004, 47, 3518-36. The primary amines of formula (2A) can then be incorporated into the syntheses of compounds of Formula I as described herein, e.g. Reaction Scheme I.

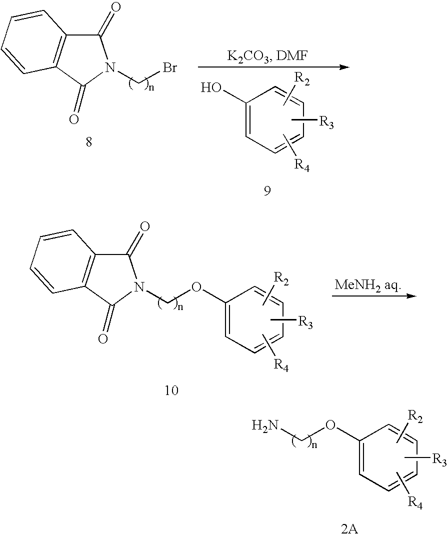

One method of synthesis of these amines includes the reaction of phthalimides (8) with substituted phenols (9), as described by Lever, W. O. Jr. J. Med. Chem. 1985, 28, 1870-4, followed by reaction with methylamine.

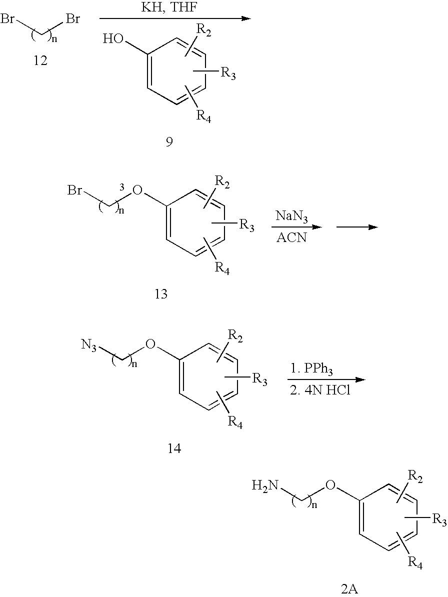

Another method of synthesis includes a reaction of excess of symmetrical α,ω-dibromoalkane (12) with substituted phenol (9) resulting in monobromide (13). Monobromide is then reacted with sodium azide to produce azide derivative (14) which is converted to primary amide by consecutive reactions, first with triphenylphosphine and then with hydrochloric acid.

### B. Synthesis of Primary Amines of Formula (2B)

Aminoalcohols of Formula (2B) (which are compounds of formula (2), see Reaction Scheme I) can be made by a variety of known synthetic methods including, but not limited, to the following references: Vigroux, A. et al, J. Med. Chem. 1995, 38, 3983-94; Erhart, P. W. et al, J. Med. Chem. 1982, 25, 1402-7. The primary amines of formula (2B) can then be incorporated into the syntheses of compounds of Formula I as described herein, e.g. Reaction Scheme I.

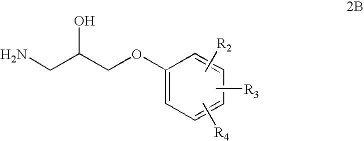

One method of synthesis of aminoalcohols includes reaction of epichlorohydrin with substituted phenols in the presence of aqueous NaOH in dioxane to compounds of formula (16). Compounds of formula (16) undergo reaction with benzylamine to produce aminoalcohols of formula (17). This particular two-step method of synthesis of substituted aminoalcohols is described by Caroon, J. M. et al. J. Med. Chem. 1981, 24, 1320-28. Final step represents de-benzylation which is accomplished by reaction with cyclohexene as hydrogen source in the presence of palladium (II) hydroxide 20% on carbon. The product is an amino-alcohol conforming to formula (2B).

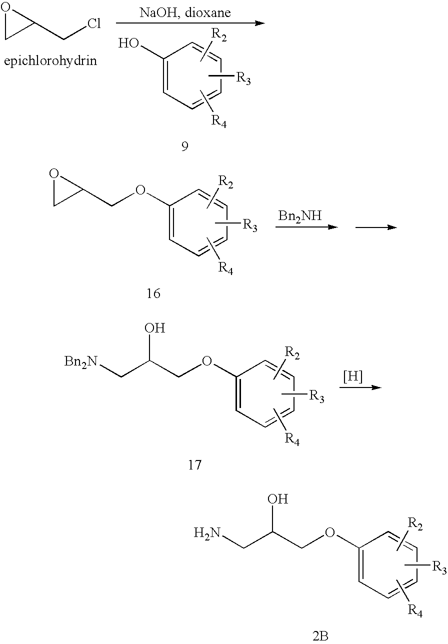

### C. Synthesis of Compounds of Formula I Using Primary Amines of Formula 2A and Formula 2B

The primary amines of formula (2A) and formula 2B can then be incorporated into the syntheses of compounds of Formula I as described herein, e.g. Reaction Scheme I. The following compounds were synthesized accordingly:
benzyl 4-oxo-3-(3-(2-(trifluoromethyl)phenoxy)propyl)-3,4-dihydroquinazolin-6-ylcarbamate;
N-(3-(2-(2,5-dichlorophenoxy)ethyl)-4-oxo-3,4-dihydroquinazolin-6-yl)-2-hydroxyacetamide;
N-(3-(3-(2,5-dichlorophenoxy)propyl)-4-oxo-3,4-dihydroquinazolin-6-yl)-2-hydroxyacetamide;
benzyl 3-(3-(2,5-dichlorophenoxy)propyl)-4-oxo-3,4-dihydroquinazolin-6-ylcarbamate;
2-hydroxy-N-(4-oxo-3-(3-(2-(trifluoromethyl)phenoxy)propyl)-3,4-dihydroquinazolin-6-yl)acetamide;
benzyl 3-(3-(2-fluorophenoxy)propyl)-4-oxo-3,4-dihydroquinazolin-6-ylcarbamate;
2-(3-(3-(2-fluorophenoxy)propyl)-4-oxo-3,4-dihydroquinazolin-6-ylamino)-2-oxoethyl acetate;
N-(3-(3-(2-fluorophenoxy)propyl)-4-oxo-3,4-dihydroquinazolin-7-yl)-2-hydroxyacetamide;
benzyl 3-(3-(2-cyanophenoxy)propyl)-4-oxo-3,4-dihydroquinazolin-6-ylcarbamate;
benzyl 3-(3-(2,5-dichlorophenoxy)propyl)-4-oxo-3,4-dihydroquinazolin-7-ylcarbamate;
N-(3-(3-(2,5-dichlorophenoxy)propyl)-4-oxo-3,4-dihydroquinazolin-7-yl)-2-hydroxyacetamide;
benzyl 4-oxo-3-(3-(o-tolyloxy)propyl)-3,4-dihydroquinazolin-6-ylcarbamate;
2-hydroxy-N-(4-oxo-3-(3-(o-tolyloxy)propyl)-3,4-dihydroquinazolin-6-yl)acetamide;
benzyl 3-(4-(2,5-dichlorophenoxy)butyl)-4-oxo-3,4-dihydroquinazolin-6-ylcarbamate;
N-(3-(4-(2,5-dichlorophenoxy)butyl)-4-oxo-3,4-dihydroquinazolin-6-yl)-2-hydroxyacetamide;
N-(3-(3-(2-chlorophenyl)propyl)-4-oxo-3,4-dihydroquinazolin-6-yl)-2-hydroxyacetamide;
benzyl 3-(2-(2-chlorophenoxy)ethyl)-4-oxo-3,4-dihydroquinazolin-6-ylcarbamate;
N-(3-(2-(2-chlorophenoxy)ethyl)-4-oxo-3,4-dihydroquinazolin-6-yl)-2-hydroxyacetamide;
benzyl 3-(3-(2-chlorophenoxy)propyl)-4-oxo-3,4-dihydroquinazolin-6-ylcarbamate;
N-(3-(3-(2-chlorophenoxy)propyl)-4-oxo-3,4-dihydroquinazolin-6-yl)-2-hydroxyacetamide;
N-(3-(3-((2,5-dichlorophenoxy)methyl)benzyl)-4-oxo-3,4-dihydroquinazolin-6-yl)-2-hydroxyacetamide;
benzyl 3-(3-((2,5-dichlorophenoxy)methyl)benzyl)-4-oxo-3,4-dihydroquinazolin-6-ylcarbamate;
N-(3-(2-(2-chlorophenoxy)ethyl)-4-oxo-3,4-dihydroquinazolin-7-yl)-2-hydroxyacetamide;
N-(3-(5-(2,5-dichlorophenoxy)pentyl)-4-oxo-3,4-dihydroquinazolin-6-yl)-2-hydroxyacetamide;
N-(3-(3-(2,3-dichlorophenoxy)propyl)-4-oxo-3,4-dihydroquinazolin-6-yl)-2-hydroxyacetamide;
N-(3-(3-(2-chloro-5-(trifluoromethyl)phenoxy)propyl)-4-oxo-3,4-dihydroquinazolin-6-yl)-2-hydroxyacetamide;
2,5-dichloro-N-(2-(6-(2-hydroxyacetamido)-4-oxoquinazolin-3(4H)-yl)ethyl)benzamide;
2-hydroxy-N-(4-oxo-3-(3-phenoxypropyl)-3,4-dihydroquinazolin-6-yl)acetamide;
N-(2-(6-(2-hydroxyacetamido)-4-oxoquinazolin-3(4H)-yl)ethyl)-2-(trifluoromethyl)benzamide;
N-(3-(3-(4-chlorophenoxy)propyl)-4-oxo-3,4-dihydroquinazolin-6-yl)-2-hydroxyacetamide;
N-(3-(2-(2,5-dichlorophenylsulfonamido)ethyl)-4-oxo-3,4-dihydroquinazolin-6-yl)-2-hydroxyacetamide;
N-(3-(3-(2,5-dichlorophenylsulfonamido)propyl)-4-oxo-3,4-dihydroquinazolin-6-yl)-2-hydroxyacetamide;
2-hydroxy-N-(4-oxo-3-(3-(2-(trifluoromethyl)phenylsulfonamido)propyl)-3,4-dihydroquinazolin-6-yl)acetamide;
N-(3-(2-(4-chlorophenoxy)ethyl)-4-oxo-3,4-dihydroquinazolin-6-yl)-2-hydroxyacetamide;
N-(3-(2-(3-chlorophenoxy)ethyl)-4-oxo-3,4-dihydroquinazolin-6-yl)-2-hydroxyacetamide; and
2-hydroxy-N-(3-(2-hydroxy-3-(o-tolyloxy)propyl)-4-oxo-3,4-dihydroquinazolin-6-yl)acetamide.

### EXAMPLE 10

### Synthesis of Compounds of Formula I in which W is N

The general synthesis set out in Reaction Scheme I was followed to produce compounds of Formula I in which W is N.

### A. Preparation of a Compound of Formula (3)

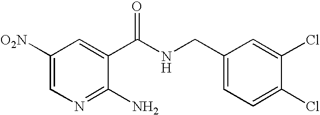

A solution of 1.0 g of 2-amino-5-nitronicotinic acid and 0.96 g of carbonyldiimidazole (CDI) in 10 mL of DMF was stirred for 1 h. Then 1.0 g of 3,4-dichlorobenzylamine was added and the mixture stirred at room temperature for 1 h. The mixture was diluted with 100 mL of water. Yellow precipitate of 2-amino-N-(3,4-dichlorobenzyl)-5-nitronicotinamide was formed, which was filtered and dried under high vacuum to be used in the next step without further purification.

### B. Preparation of a Compound of Formula (4)

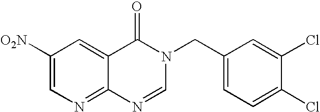

To a suspension of 0.3 g of 2-amino-N-(3,4-dichlorobenzyl)-5-nitronicotinamide in 20 mL of trimethylorthoformate in a Biotage microwave vial was added 1 drop of acetic acid. The mixture was subjected to microwave irradiation, maintaining an internal reaction temperature of 200° C. for 30 minutes. The solvent was removed in vacuo to provide crude 3-(3,4-dichlorobenzyl)-6-nitropyrido[2,3-d]pyrimidin-4(3H)-one as a light-yellow solid.

### C. Preparation of a Compound of Formula (5)

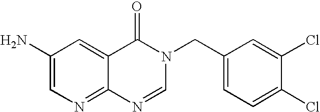

To a solution of 0.5 g of crude 3-(3,4-dichlorobenzyl)-6-nitropyrido[2,3-d]pyrimidin-4(3H)-one was suspended in 70 mL of MeOH and Raney Nickel was added, followed by 0.35 mL of hydrazine hydrate in small portions. The mixture was heated to 55° C. for 5 min. At that time 1 mL of acetic acid was added, followed by 0.15 mL of hydrazine and additional 5 minutes of heating. The mixture was decanted and filtered to remove the solids that include Raney Nickel. The filtrate was cooled and concentrated in vacuo to a fraction of original volume, at which point precipitate formed. It was filtered and dried to provide 6-amino-3-(3,4-dichlorobenzyl)pyrido[2,3-d]pyrimidin-4(3H)-one as solid, which was used without further purification.

### D. Preparation of a Compound of Formula I

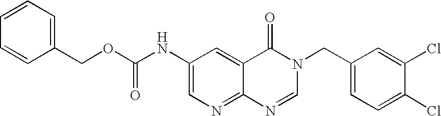

To a solution of 32 mg of 6-amino-3-(3,4-dichlorobenzyl)pyrido[2,3-d]pyrimidin-4(3H)-one in acetonitrile was added 26 mg of diisopropylethylamine followed by 35 mg of benzyl chloroformate (CbzCl). The solvent was removed in vacuo and the product isolated by silica gel chromatography followed by reverse-phase chromatography to provide the compound of Formula I, benzyl 3-(3,4-dichlorobenzyl)-4-oxo-3,4-dihydropyrido[2,3-d]pyrimidin-6-ylcarbamate.

MS (ESI, m/z): found 454.5 [M+H]+.

### E. Preparation of a Compound of Formula I

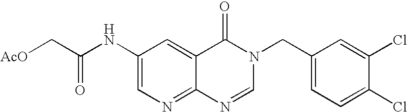

A solution containing acetylglycolic acid (35 mg) and carbonyldiimidazole (0.11 mmol) in 5 mL of dichloroethane was stirred at room temperature for 0.5 hr, then 6-amino-3-(3,4-dichlorobenzyl)pyrido[2,3-d]pyrimidin-4(3H)-one (30 mg) was added and stirring was continued at 70° C. for 3 h. The reaction mixture was concentrated and subjected to reverse-phase chromatography using C(18) column with water and acetonitrile as eluents to afford the product, 2-(3-(3,4-dichlorobenzyl)-4-oxo-3,4-dihydropyrido[2,3-d]pyrimidin-6-ylamino)-2-oxoethyl acetate.

MS (APCI, m/z): found 420.6 [M+H⁺].

### F. Preparation of a Compound of Formula I

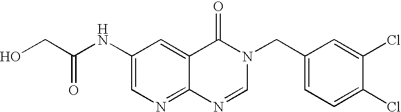

To a solution of 17 mg of 2-(3-(3,4-dichlorobenzyl)-4-oxo-3,4-dihydropyrido[2,3-d]pyrimidin-6-ylamino)-2-oxoethyl acetate in 3 mL of methanol was added an aqueous solution of KOH (10 mg in 1 mL) followed by additional 3 mL of ethanol. After 3 minutes of stirring at room temperature saturated aqueous solution of Na₂CO₃ was added. The resulting mixture was extracted with CH₂Cl₂, dried over Na₂SO₄, and concentrated to provide the product, N-(3-(3,4-dichlorobenzyl)-4-oxo-3,4-dihydropyrido[2,3-d]pyrimidin-6-yl)-2-hydroxyacetamide.

MS (ESI, m/z): 379.0 [M+H⁺].

¹H NMR (δ, MeOH-d₃, 400 MHz): 9.03 (s, 1H); 8.82 (s, 1H); 8.22 (s, 1H); 7.39-7.10 (m, 3H); 5.05 (s, 2H); and 4.08 (s, 2H).

### EXAMPLE 11

### Preparation of Further Compounds of Formula I

Following the procedures set out in Examples 4 through 10 above, but optionally substituting other starting materials as may be determined by inspection of the final compound, the following compounds of Formula I were prepared:
2-(3-(3,4-difluorobenzyl)-4-oxo-3,4-dihydroquinazolin-6-ylamino)-2-oxoethyl acetate;
N-(3-(3,4-difluorobenzyl)-4-oxo-3,4-dihydroquinazolin-6-yl)-2-hydroxyacetamide;
2-(3-(4-chlorobenzyl)-4-oxo-3,4-dihydroquinazolin-6-ylamino)-2-oxoethyl acetate;
N-(3-(4-chlorobenzyl)-4-oxo-3,4-dihydroquinazolin-6-yl)-2-hydroxyacetamide;
N-(3-(4-chlorobenzyl)-4-oxo-3,4-dihydroquinazolin-6-yl)-2-phenoxyacetamide;
2-(3-(4-chloro-3-(trifluoromethyl)benzyl)-4-oxo-3,4-dihydroquinazolin-6-ylamino)-2-oxoethyl acetate;
N-(3-(4-chloro-3-(trifluoromethyl)benzyl)-4-oxo-3,4-dihydroquinazolin-6-yl)-2-hydroxyacetamide;

benzyl 3-(3,4-dichlorobenzyl)-2-methyl-4-oxo-3,4-dihydroquinazolin-7-ylcarbamate;
benzyl 3-(1-(3,4-dichlorophenyl)ethyl)-4-oxo-3,4-dihydroquinazolin-7-ylcarbamate;
2-(3-(1-(3,4-dichlorophenyl)ethyl)-4-oxo-3,4-dihydroquinazolin-7-ylamino)-2-oxoethyl acetate;
N-(3-(1-(3,4-dichlorophenyl)ethyl)-4-oxo-3,4-dihydroquinazolin-7-yl)-2-hydroxyacetamide;
benzyl 3-(3-methoxybenzyl)-4-oxo-3,4-dihydroquinazolin-6-ylcarbamate;
2-(3-(3-chloro-4-fluorobenzyl)-4-oxo-3,4-dihydroquinazolin-6-ylamino)-2-oxoethyl acetate;
benzyl 3-(4-fluoro-3-(trifluoromethyl)benzyl)-4-oxo-3,4-dihydroquinazolin-6-ylcarbamate;
2-(3-(4-fluoro-3-(trifluoromethyl)benzyl)-4-oxo-3,4-dihydroquinazolin-6-ylamino)-2-oxoethyl acetate;
N-(3-(4-fluoro-3-(trifluoromethyl)benzyl)-4-oxo-3,4-dihydroquinazolin-6-yl)-2-hydroxyacetamide;
N-(3-(3,4-dichlorobenzyl)-4-oxo-3,4-dihydroquinazolin-6-yl)acetamide;
2-hydroxy-N-(4-oxo-3-(3-phenoxyphenyl)-3,4-dihydroquinazolin-6-yl)acetamide;
N-(3-(3,4-difluorobenzyl)-4-oxo-3,4-dihydroquinazolin-6-yl)acetamide;
N-(4-oxo-3-(4-(trifluoromethyl)benzyl)-3,4-dihydroquinazolin-6-yl)acetamide;
benzyl 3-(3,4-dimethylbenzyl)-4-oxo-3,4-dihydroquinazolin-6-ylcarbamate;
2-(3-(3,4-dimethylbenzyl)-4-oxo-3,4-dihydroquinazolin-6-ylamino)-2-oxoethyl acetate;
N-(3-(3,4-dimethylbenzyl)-4-oxo-3,4-dihydroquinazolin-6-yl)-2-hydroxyacetamide;
N-(3-(1-(3,4-dichlorophenyl)ethyl)-4-oxo-3,4-dihydroquinazolin-6-yl)-2-hydroxyacetamide;
2-oxo-2-(4-oxo-3-(3-phenylpropyl)-3,4-dihydroquinazolin-6-ylamino)ethyl acetate;
2-(3-(1-(3,4-dichlorophenyl)propyl)-4-oxo-3,4-dihydroquinazolin-6-ylamino)-2-oxoethyl acetate;
N-(3-(1-(3,4-dichlorophenyl)propyl)-4-oxo-3,4-dihydroquinazolin-6-yl)-2-hydroxyacetamide;
N-(3-benzyl-4-oxo-3,4-dihydroquinazolin-6-yl)-2-hydroxyacetamide;
2-hydroxy-N-(4-oxo-3-(3-phenylpropyl)-3,4-dihydroquinazolin-6-yl)acetamide;
2-hydroxy-N-(4-oxo-3-phenethyl-3,4-dihydroquinazolin-6-yl)acetamide;
2-oxo-2-(4-oxo-3-(2-(trifluoromethyl)benzyl)-3,4-dihydroquinazolin-6-ylamino)ethyl acetate;
N-(3-(biphenyl-3-ylmethyl)-4-oxo-3,4-dihydroquinazolin-6-yl)-2-hydroxyacetamide;
benzyl 3-(1-(3,4-dichlorophenyl)propyl)-4-oxo-3,4-dihydroquinazolin-6-ylcarbamate;
2-hydroxy-N-(3-(naphthalen-2-ylmethyl)-4-oxo-3,4-dihydroquinazolin-6-yl)acetamide;
benzyl 3-(naphthalen-2-ylmethyl)-4-oxo-3,4-dihydroquinazolin-6-ylcarbamate;
2-hydroxy-N-(3-(naphthalen-2-ylmethyl)-4-oxo-3,4-dihydroquinazolin-7-yl)acetamide;
(R)—N-(3-(1-(4-chloro-3-(trifluoromethyl)phenyl)ethyl)-4-oxo-3,4-dihydroquinazolin-6-yl)-2-hydroxyacetamide;
(S)—N-(3-(1-(3,4-dichlorophenyl)ethyl)-4-oxo-3,4-dihydroquinazolin-7-yl)-2-hydroxyacetamide;
(R)—N-(3-(1-(3,4-dichlorophenyl)ethyl)-4-oxo-3,4-dihydroquinazolin-7-yl)-2-hydroxyacetamide;
N-(3-(4-chlorophenethyl)-4-oxo-3,4-dihydroquinazolin-6-yl)-2-hydroxyacetamide;
N-(3-(3-chlorophenethyl)-4-oxo-3,4-dihydroquinazolin-6-yl)-2-hydroxyacetamide;
N-(3-(2,4-dichlorophenethyl)-4-oxo-3,4-dihydroquinazolin-6-yl)-2-hydroxyacetamide; and
benzyl 4-oxo-3-(3-(trifluoromethyl)benzyl)-3,4-dihydroquinazolin-6-ylcarbamate.

### EXAMPLE 12

### Characterization of Stearoyl-CoA Desaturase Inhibitor

### Materials and Methods

### Materials

[³H]stearoyl CoA and sterculic acid were obtained from PerkinElmer and Planta Piloto de Quimica Fina, respectively. Commercial sources of other reagents are listed below:

| Material | Company |
|---|---|
| [³H]H₂O | PerkinElmer |
| Stearoyl CoA | Sigma |
| CoA | Sigma |
| NADH | Sigma |
| Tris, 1 M | Invitrogen |
| MgCl2 | Sigma |
| BHT | Sigma |
| BSA | Sigma |
| DMSO | Sigma |
| ATP | Sigma |
| 96-well plates | Corning |
| Bio-Beads SM-2 | Bio-Rad |

### Preparation of Rat Liver Microsomes

The rat liver microsomes were collected according to the procedure described in Ozols (1990) Methods Enzm, 182:225.

### In Vivo Experiment (Liver Perfusion and Collection)

Male Spraque Dawley Rats were placed on regimented fasting protocol for one week to stimulate SCD enzymatic activity. 48-hour periods were alternated between feeding and fasting to induce and down-regulate SCD activity with SCD activity being induced via carbohydrate rich diet prior to liver perfusion and collection.

The rats were anesthetized with Isoflurane inhalation anesthetic, the liver perfused with cold phosphate buffered saline (PBS), weighed, and chilled in cold homogenization buffer (250 mM sucrose, 10 mM Tris, 1 mM EDTA, pH 7.6.

The livers were finely minced and placed in homogenization tube. 40 mL of homogenization buffer was added to the homogenization tube and the liver homogenized and centrifuged in a pre-chilled SLA-600 TC at 800 G rotor for 10 min at 4° C.

Following centrifugation, the supernatant was collected and the pellet removed and discarded. The supernatant was centrifuge at 10,000 G for 35 minutes. Following centrifugation, the supernatant was collect and the pellet discarded. The supernatant was then centrifuged in a pre-chilled 45-Ti rotor at 130,000 G (41,000 RPM) for 90 minutes at 4° C.

### In Vitro (Microsomal Collection)

The supernatant was then aspirated off and the collected microsomal pellet washed in 25 mL of Glycerol PBS (1×PBS 7.4, 20% Glycerol) and resuspended in 4-5 volumes of Glycerol PBS.

The protein concentration of the microsomal preparation was determined by BCA assay (Pierce) and the microsomes were aliquoted and stored at −80° C.

### Preparation of Hydrophobic Beads

Biobeads were ground to a smaller size in a mortar and pestle and resuspended in 3.6% TCA. The beads were then filtered through 300 μM mesh.

### Stock Solutions

Stock solutions and their storage conditions are listed below:

| Solution | Storage condition |
|---|---|
| 20 mg/ml Stearoyl CoA | −80° C. |
| 2.8 mCi/ml [³H]Stearoyl CoA | −80° C. |
| CoA | freshly prepared |
| Sterculic acid | freshly prepared |
| 0.2 M NADH | −80° C. |
| 1 M Tris, pH 7.2 | room temperature |
| 1 M MgCl2 | room temperature |
| 100 mM ATP | −20° C. |
| 10% BSA | 4° C. |
| 10-20 mg/ml microsome | −80° C. |

### The SCD Assay Buffer

SCD was determined in the desaturase assay buffer. This assay buffer contained 0.1 M Tris buffer, pH 7.2, 2 mM NADH, 4.8 mM ATP, 0.5 mM CoA, 4.8 mM MgCl2, and 0.1% BSA.

The Procedure for the SCD Assay (Adapted from Talamo and Bloch (1969) Analytical Biochemistry 29:300-304)

1 μl of each compound of Formula I was added to an assay plate by a low volume (0.5-10 μL) multichannel pipette. A DMSO control was also prepared. The microsomes were quickly thawed and added to assay buffer so that a concentration of 0.4 mg/ml was achieved (0.2 mg/ml assay final). 50 μl of the microsome suspension in assay buffer was then added into each well in the compound assay plate, the plate was covered, and the microsomes preincubated with the compounds for 30 minutes on the orbital shaker, 50-75 rpm, at room temperature.

After preincubation, the reaction was initiated by the addition of 501 of substrate solution (20 μM Stearoyl CoA, [3H]Stearoyl CoA, 74 nCi) to the preincubated microsomes/compound suspensions in MilliQ (Millipore) H₂O. The reaction mixtures were then incubated for 45 minutes on the orbital shaker at 50-75 rpm at room temperature.

The reaction was terminated by the addition of 10 μl of 21% trichloroacetic acid (TCA) to the reaction mixture followed incubation on the orbital shaker for 30 minutes at 50-75 rpm at room temperature followed by centrifugation for 5 minutes at 3700 rpm.

50 μl of a 6% Bio-Bead suspension in H₂O was added to the reaction mixture and the assay plate was sealed. The Bio-Bead mixture was incubated on the orbital shaker for 1 hour, 100-150 rpm at room temperature, and then the mixture was centrifuged at 2000 g for 5 minutes to pellet the Bio-Beads.

25 μl of the supernatant was harvested from each well and transferred to a detection plate. 100 μl of OptiPhase SuperMix scinitillation cocktail (containing sufficient NaOH to neutralize the TCA) was added and the solutions mixed by vigorous shaking (300-400 rpm) for 5 minutes at room temperature. The radioactivity was counted in a MicroBeta scintillation counter in order to determine the activity and IC₅₀ values for the compounds of Formula I. Table 1 presents the IC₅₀ data for a number of compounds of the invention for which the IC₅₀ as determined in the above assay was less than 30 μM.

TABLE 1

| NUMBER | NAME | IC₅₀ μM |
|---|---|---|
| I. | N-{3-[(3,4-dichlorophenyl)methyl]-4-oxo(3-hydroquinazolin- | 0.16 |
| | 6-yl)}(phenylmethoxy)carboxamide | |
| II. | N-{3-[(3,4-dichlorophenyl)methyl]-4-oxo(3-hydroquinazolin- | 2.41 |
| | 6-yl)}-3-phenylpropanamide | |
| III. | N-{3-[(3,4-dichlorophenyl)methyl]-4-oxo-3- | 5.31 |
| | hydroquinazolin-6-yl}hexanamide | |
| IV. | N-{3-[(3,4-dichlorophenyl)methyl]-4-oxo(3- | 3.59 |
| | hydroquinazolin-6-yl)}(pentylamino)carboxamide | |
| V. | N-{3-[(3,4-dichlorophenyl)methyl]-4-oxo(3- | 3.18 |
| | hydroquinazolin-6-yl)}[benzylamino]carboxamide | |
| VI. | N-{3-[(3,4-dichlorophenyl)methyl]-2-methyl-4-oxo(3- | 20 |
| | hydroquinazolin-6-yl)}(phenylmethoxy)carboxamide | |
| VII | N-{3-[(3,4-dichlorophenyl)methyl]-4-oxo(3- | 7.9 |
| | hydroquinazolin-6-yl)}(2-methylpropoxy)carboxamide | |
| VIII. | N-{3-[(4-chlorophenyl)methyl]-4-oxo(3-hydroquinazolin-6- | 0.19 |
| | yl)}(phenylmethoxy)carboxamide | |
| IX. | N-{3-[(3,4-dichlorophenyl)methyl]-4-oxo(3- | 14 |
| | hydroquinazolin-6-yl)}(methylthoxy)carboxamide | |
| X. | N-{3-[(3,4-dichlorophenyl)methyl]-4-oxo(3- | 1.5 |
| | hydroquinazolin-6-yl)}prop-2-enyloxycarboxamide | |
| XI. | N-{3-[(3,4-dichlorophenyl)methyl]-4-oxo(3- | 2.7 |
| | hydroquinazolin-6-yl)}(4-methylphenoxy)carboxamide | |
| XII. | N-{3-[(3,4-dichlorophenyl)methyl]-4-oxo(3- | 13 |
| | hydroquinazolin-6-yl)}(4-fluorophenoxy)carboxamide | |
| XIII. | N-{3-[(3,4-dichlorophenyl)methyl]-4-oxo(3- | 4.7 |
| | hydroquinazolin-6-yl)}methoxycarboxamide | |
| XIV. | N-{3-[(4-chlorophenyl)methyl]-4-oxo(3-hydroquinazolin-6- | 25.7 |
| | yl)}prop-2-enyloxycarboxamide | |
| XV. | N-{3-[(3,4-dichlorophenyl)methyl]-4-oxo(3- | 0.22 |
| | hydroquinazolin-6-yl)}-2-phenoxyacetamide | |
| XVI. | N-{3-[2-(4-chlorophenyl)ethyl]-4-oxo(3-hydroquinazolin-6- | 0.47 |
| | yl)}(phenylmethoxy)carboxamide | |
| XVII. | N-{3-[(3,4-dichlorophenyl)methyl]-4-oxo(3- | 2.98 |
| | hydroquinazolin-6-yl)}-2-(4-methoxyphenoxy)acetamide | |
| XVIII. | N-{3-[(3,4-dichlorophenyl)methyl]-4-oxo(3- | 2.10 |
| | hydroquinazolin-6-yl)}-2-(4-fluorophenoxy)acetamide | |
| XIX. | 2-(acetylamino)-N-{3-[(3,4-dichlorophenyl)methyl]-4- | 11.7 |
| | oxo(3-hydroquinazolin-6-yl)}acetamide | |
| XX. | N-{3-[(3,4-dichlorophenyl)methyl]-4-oxo(3- | 2.92 |
| | hydroquinazolin-6-yl)}-2-(3,5-dimethylphenoxy)acetamide | |
| XXI. | N-{3-[(3,4-dichlorophenyl)methyl]-4-oxo(3- | 3.45 |
| | hydroquinazolin-6-yl)}-2-(2,6-dimethylphenoxy)acetamide | |
| XXII. | N-{3-[(3,4-dichlorophenyl)methyl]-4-oxo(3- | 5.7 |
| | hydroquinazolin-6-yl)}-2-(4-methylphenoxy)acetamide | |
| XXIII. | N-{3-[(3,4-dichlorophenyl)methyl]-4-oxo(3- | 0.17 |
| | hydroquinazolin-6-yl)}(3-pyridylmethoxy)carboxamide | |
| XXIV. | N-{3-[(4-fluorophenyl)methyl]-4-oxo(3-hydroquinazolin-6- | 0.30 |
| | yl)}(phenylmethoxy)carboxamide | |
| XXV. | N-{3-[(3,4-dichlorophenyl)methyl]-4-oxo(3- | 0.78 |
| | hydroquinazolin-6-yl)}-2-(2-methoxyphenoxy)acetamide | |
| XXVI. | N-{3-[(3,4-dichlorophenyl)methyl]-4-oxo(3- | 3.69 |
| | hydroquinazolin-6-yl)}-2-(2-fluorophenoxy)acetamide | |
| XXVII. | N-{3-[(3,4-dichlorophenyl)methyl]-4-oxo(3- | 2.01 |
| | hydroquinazolin-6-yl)}-2-(3-fluorophenoxy)acetamide | |
| XXVIII. | N-{3-[(3,4-dichlorophenyl)methyl]-4-oxo(3- | 3.43 |
| | hydroquinazolin-6-yl)}-2-(2,4-difluorophenoxy)acetamide | |
| XXIX. | N-{3-[(3,4-dichlorophenyl)methyl]-4-oxo(3- | 1.98 |
| | hydroquinazolin-6-yl)}-2-(2-chlorophenoxy)acetamide | |
| XXX. | N-{3-[(3,4-dichlorophenyl)methyl]-4-oxo(3- | 3.78 |
| | hydroquinazolin-6-yl)}-2-(2,3-dichlorophenoxy)acetamide | |
| XXXI. | N-{3-[(3,4-dichlorophenyl)methyl]-4-oxo(3- | 15.0 |
| | hydroquinazolin-6-yl)}-2-(2-nitrophenoxy)acetamide | |
| XXXII. | N-{3-[(3,4-dichlorophenyl)methyl]-4-oxo(3- | 2.85 |
| | hydroquinazolin-6-yl)}-2-(2-naphthyloxy)acetamide | |
| XXXIII. | N-{3-[(3,4-dichlorophenyl)methyl]-4-oxo(3- | 6.88 |
| | hydroquinazolin-6-yl)}-3-phenoxypropanamide | |
| XXXIV. | N-{3-[(3,4-dichlorophenyl)methyl]-4-oxo(3- | 2.1 |
| | hydroquinazolin-6-yl)}-2-(2-chlorophenoxy)propanamide | |
| XXXV. | 2-benzo[c]1,2,5-thiadiazol-4-yloxy-N-{3-[(3,4- | 21 |
| | dichlorophenyl)methyl]-4-oxo(3-hydroquinazolin-6- | |
| | yl)}acetamide | |
| XXXVI. | N-{3-[(3,4-dichlorophenyl)methyl]-4-oxo(3- | 3.1 |
| | hydroquinazolin-6-yl)}-2-(3-pyridyloxy)acetamide | |
| XXXVII. | N-{3-[(4-chlorophenyl)methyl]-4-oxo(3-hydroquinazolin-6- | 7.3 |
| | yl)}-2-(4-pyridylthio)acetamide | |
| XXXVIII. | N-{3-[(4-bromophenyl)methyl]-4-oxo(3-hydroquinazolin-6- | 15 |
| | yl)}-2-phenoxyacetamide | |
| XXXIX. | N-{3-[(4-chloro-3-fluorophenyl)methyl]-4-oxo(3- | 6.1 |
| | hydroquinazolin-6-yl)}-2-phenoxyacetamide | |
| XL. | N-{3-[(3-chloro-4-fluorophenyl)methyl]-4-oxo(3- | 9.5 |
| | hydroquinazolin-6-yl)}-2-phenoxyacetamide | |
| XLI. | N-[3-(benzo[b]thiophen-6-ylmethyl)-4-oxo(3- | 8 |
| | hydroquinazolin-6-yl)]-2-phenoxyacetamide | |
| XLII. | N-{3-[(3,4-dichlorophenyl)methyl]-4-oxo(3- | 2.17 |
| | hydroquinazolin-6-yl)}-4-oxo-4-phenylbutanamide | |
| XLIII. | N-{3-[(4-chlorophenyl)methyl]-4-oxo(3-hydroquinazolin-6- | 6.4 |
| | yl)}-2-(3-fluorophenoxy)acetamide | |
| XLIV. | N-{3-[(4-chlorophenyl)methyl]-4-oxo(3-hydroquinazolin-7- | 1.6 |
| | yl)}-2-phenoxyacetamide | |
| XLV. | N-[3-(benzo[b]thiophen-5-ylmethyl)-4-oxo(3- | 0.86 |
| | hydroquinazolin-6-yl)](phenylmethoxy)carboxamide | |
| XLVI. | N-{3-[(3-chloro-4-fluorophenyl)methyl]-4-oxo(3- | 0.45 |
| | hydroquinazolin-6-yl)}(phenylmethoxy)carboxamide | |
| XLVII. | (N-{3-[(3,4-dichlorophenyl)methyl]-4-oxo-3- | 0.10 |
| | hydroquinazolin-6-yl}carbamoyl)methyl acetate | |
| XLVIII. | N-{3-[(4-chlorophenyl)methyl]-4-oxo(3-hydroquinazolin-7- | 0.09 |
| | yl)}(phenylmethoxy)carboxamide | |
| XLIX. | N-{3-[(4-chlorophenyl)methyl]-4-oxo(3-hydroquinazolin-7- | 2.5 |
| | yl)}-2-(3-pyridyloxy)acetamide | |
| L. | N-{3-[(3,4-dichlorophenyl)methyl]-4-oxo(3- | 1.44 |
| | hydroquinazolin-7-yl)}-2-(2-methoxyphenoxy)acetamide | |
| LI. | N-{3-[(3,4-dichlorophenyl)methyl]-4-oxo(3- | 1.45 |
| | hydroquinazolin-7-yl)}-2-(3-pyridyloxy)acetamide | |
| LII. | N-{3-[(3,4-dichlorophenyl)methyl]-4-oxo(3- | 0.05 |
| | hydroquinazolin-7-yl)}(phenylmethoxy)carboxamide | |
| LIII. | N-{3-[(3,4-dichlorophenyl)methyl]-4-oxo(3- | 1.69 |
| | hydroquinazolin-7-yl)}-2-phenoxyacetamide | |
| LIV. | N-{3-[(3,4-dichlorophenyl)methyl]-4-oxo(3- | 2.56 |
| | hydroquinazolin-6-yl)}-2-(4-pyridylthio)acetamide | |
| LV. | N-{3-[(3,4-dichlorophenyl)methyl]-4-oxo(3- | 5.42 |
| | hydroquinazolin-6-yl)}-2-pyrimidin-2-ylthioacetamide | |
| LVI. | N-{3-[(3,4-dichlorophenyl)methyl]-4-oxo(3- | 2.81 |
| | hydroquinazolin-6-yl)}-2-(4-chlorophenylthio)acetamide | |
| LVII. | N-{3-[(4-chlorophenyl)methyl]-4-oxo(3-hydroquinazolin-6- | 12.0 |
| | yl)}[5-(imidazolylmethyl)(2-furyl)]carboxamide | |
| LVIII. | N-{3-[(3-chlorophenyl)methyl]-4-oxo(3-hydroquinazolin-7- | 0.25 |
| | yl)}(phenylmethoxy)carboxamide | |
| LIX. | N-{3-[(3-chlorophenyl)methyl]-4-oxo(3-hydroquinazolin-7- | 0.28 |
| | yl)}(3-pyridylmethoxy)carboxamide | |
| LX. | N-{3-[(3,4-dichlorophenyl)methyl]-4-oxo(3- | 6.6 |
| | hydroquinazolin-6-yl)}-2-(phenylmethylthio)acetamide | |
| LXI. | N-{3-[(3,4-dichlorophenyl)methyl]-4-oxo(3- | 0.70 |
| | hydroquinazolin-7-yl)}[(2- | |
| | methoxyphenyl)methoxy]carboxamide | |
| LXII. | N-{3-[(3,4-dichlorophenyl)methyl]-4-oxo(3- | 0.23 |
| | hydroquinazolin-6-yl)}-2-hydroxyacetamide | |
| LXIII. | N-{3-[(3,4-dichlorophenyl)methyl]-4-oxo(3- | 0.44 |
| | hydroquinazolin-6-yl)}(2-furylmethoxy)carboxamide | |
| LXIV. | (N-{3-[(3,4-dichlorophenyl)methyl]-4-oxo-3- | 0.18 |
| | hydroquinazolin-7-yl}carbamoyl)methyl acetate | |
| LXV. | (N-{3-[(3-chlorophenyl)methyl]-4-oxo-3-hydroquinazolin- | 27.1 |
| | 7-yl}carbamoyl)methyl acetate | |
| LXVI. | (N-{3-[(4-chlorophenyl)methyl]-4-oxo-3-hydroquinazolin- | 16.4 |
| | 7-yl}carbamoyl)methyl acetate | |
| LXVII. | N-{3-[(3,4-dichlorophenyl)methyl]-4-oxo(3- | 0.88 |
| | hydroquinazolin-7-yl)}(2-methoxyethoxy)carboxamide | |
| LXVIII. | N-{3-[(3-chlorophenyl)methyl]-4-oxo(3-hydroquinazolin-7- | 2.69 |
| | yl)}-2-hydroxyacetamide | |
| LXIX. | N-{3-[(3-fluorophenyl)methyl]-4-oxo(3-hydroquinazolin-7- | 0.84 |
| | yl)}(phenylmethoxy)carboxamide | |
| LXX. | N-{3-[(3,4-dichlorophenyl)methyl]-4-oxo(3- | 3.22 |
| | hydroquinazolin-6-yl)}(4-chlorophenoxy)carboxamide | |
| LXXI. | N-{3-[(3,4-dichlorophenyl)methyl]-4-oxo(3- | 4.38 |
| | hydroquinazolin-6-yl)}(2-phenylethoxy)carboxamide | |
| LXXII. | (N-{3-[(3-fluorophenyl)methyl]-4-oxo-3-hydroquinazolin- | 0.29 |
| | 7-yl}carbamoyl)methyl acetate | |
| LXXIII. | N-{3-[(3-chloro-4-fluorophenyl)methyl]-4-oxo(3- | 0.29 |
| | hydroquinazolin-7-yl)}(phenylmethoxy)carboxamide | |
| LXXIV. | N-{3-[(3-chloro-4-fluorophenyl)methyl]-4-oxo(3- | 0.12 |
| | hydroquinazolin-7-yl)}(3-pyridylmethoxy)carboxamide | |
| LXXV. | (N-{3-[(3-chloro-4-fluorophenyl)methyl]-4-oxo-3- | 1.09 |
| | hydroquinazolin-7-yl}carbamoyl)methyl acetate | |
| LXXVI. | N-{3-[(3-fluorophenyl)methyl]-4-oxo(3-hydroquinazolin-7- | 1.34 |
| | yl)}(3-pyridylmethoxy)carboxamide | |
| LXXVII. | N-{3-[(4-chloro-3-fluorophenyl)methyl]-4-oxo(3- | 0.25 |
| | hydroquinazolin-7-yl)}(3-pyridylmethoxy)carboxamide | |
| LXXVIII. | (N-{3-[(4-chloro-3-fluorophenyl)methyl]-4-oxo-3- | 1.02 |
| | hydroquinazolin-7-yl}carbamoyl)methyl acetate | |
| LXXIX. | N-{3-[(3,4-dichlorophenyl)methyl]-4-oxo(3- | 1.00 |
| | hydroquinazolin-6-yl)}(4-pyridylmethoxy)carboxamide | |
| LXXX. | N-{3-[(3,4-dichlorophenyl)methyl]-4-oxo(3- | 0.67 |
| | hydroquinazolin-6-yl)}[(3- | |
| | cyanophenyl)methoxy]carboxamide | |
| LXXXI. | N-{3-[(4-chlorophenyl)methyl]-4-oxo(3-hydroquinazolin-7- | 3.12 |
| | yl)}-2-hydroxyacetamide | |
| LXXXII. | N-{3-[(3,4-dichlorophenyl)methyl]-4-oxo(3- | 0.35 |
| | hydroquinazolin-7-yl)}-2-hydroxyacetamide | |
| LXXXIII. | N-{3-[(4-chloro-3-fluorophenyl)methyl]-4-oxo(3- | 0.21 |
| | hydroquinazolin-7-yl)}(phenylmethoxy)carboxamide | |
| LXXXIV. | N-{3-[(3,4-dichlorophenyl)methyl]-4-oxo(3- | 11.0 |
| | hydroquinazolin-6-yl)}{2- | |
| | [methylbenzylamino]ethoxy}carboxamide | |
| LXXXV. | N-{3-[(3,4-dichlorophenyl)methyl]-4-oxo(3- | 2.97 |
| | hydroquinazolin-6-yl)}(cyclopropylmethoxy)carboxamide | |
| LXXXVI. | (N-{3-[(3,4-dichlorophenyl)methyl]-4-oxo-3- | 0.36 |
| | hydroquinazolin-6-yl}carbamoyl)methyl 2- | |
| | (dimethylamino)acetate | |
| LXXXVII$$ | N-{3-[(3-chloro-4-fluorophenyl)methyl]-4-oxo(3- | 2.32 |
| | hydroquinazolin-7-yl)}-2-hydroxyacetamide | |
| LXXXVII$$ | ethyl 2-(N-{3-[(3,4-dichlorophenyl)methyl]-4-oxo-3- | 2.23 |
| | hydroquinazolin-6-yl}carbamoyloxy)acetate | |
| LXXXIX. | N-{3-[(3,4-dichlorophenyl)methyl]-4-oxo(3- | 0.78 |
| | hydroquinazolin-6-yl)}(2-pyridylmethoxy)carboxamide | |
| XC. | (N-{3-[(3,4-dichlorophenyl)methyl]-4-oxo-3- | 0.31 |
| | hydroquinazolin-7-yl}carbamoyl)methyl 2- | |
| | (dimethylamino)acetate | |
| XCI. | ethyl 2-(N-{3-[(3,4-dichlorophenyl)methyl]-4-oxo-3- | 3.77 |
| | hydroquinazolin-7-yl}carbamoyloxy)acetate | |
| XCII. | N-{3-[(3,4-dichlorophenyl)methyl]-4-oxo(3- | 11.7 |
| | hydroquinazolin-6-yl)}[2-(2- | |
| | methoxyethoxy)ethoxy]carboxamide | |
| XCIII. | N-{3-[(3,4-dichlorophenyl)methyl]-4-oxo(3- | 6.20 |
| | hydroquinazolin-6-yl)}[(2- | |
| | methoxyethyl)amino]carboxamide | |
| XCIV. | [N-(4-oxo-3-{[3-(trifluoromethyl)phenyl]methyl}-3- | 1.70 |
| | hydroquinazolin-6-yl)carbamoyl]methyl acetate | |
| XCV. | 2-hydroxy-N-(4-oxo-3-{[3- | 1.38 |
| | (trifluoromethyl)phenyl]methyl}(3-hydroquinazolin-6- | |
| | yl))acetamide | |
| XCVI. | [N-(4-oxo-3-{[4-(trifluoromethyl)phenyl]methyl}-3- | 2.74 |
| | hydroquinazolin-6-yl)carbamoyl]methyl acetate | |
| XCVII. | 2-hydroxy-N-(4-oxo-3-{[4- | 3.16 |
| | (trifluoromethyl)phenyl]methyl}(3-hydroquinazolin-6- | |
| | yl))acetamide | |
| XCVIII. | (N-{3-[(4-chloro-3-fluorophenyl)methyl]-4-oxo-3- | 1.32 |
| | hydroquinazolin-6-yl}carbamoyl)methyl acetate | |
| XCIX. | N-{3-[(4-chloro-3-fluorophenyl)methyl]-4-oxo(3- | 1.16 |
| | hydroquinazolin-6-yl)}-2-hydroxyacetamide | |
| C. | (N-{3-[(4-methoxyphenyl)methyl]-4-oxo-3- | 3.50 |
| | hydroquinazolin-6-yl}carbamoyl)methyl acetate | |
| CI. | 2-hydroxy-N-{3-[(4-methoxyphenyl)methyl]-4-oxo(3- | 8.22 |
| | hydroquinazolin-6-yl)}acetamide | |
| CII. | (N-{3-[(3,4-difluorophenyl)methyl]-4-oxo-3- | 7.64 |
| | hydroquinazolin-7-yl}carbamoyl)methyl acetate | |
| CIII. | N-{3-[(3,4-difluorophenyl)methyl]-4-oxo(3- | 4.39 |
| | hydroquinazolin-7-yl)}-2-hydroxyacetamide | |

### EXAMPLE 10

Characterization of Stearoyl-CoA Desaturase Inhibitor

The procedures of Example 7 were followed to in order to determine the activity and IC₅₀ values for example compounds of Formula I. Table 2 presents the IC₅₀ data for a number of compounds of the invention for which the IC₅₀ as determined in the above assay was less than 30 μM.

TABLE 2

| | | IC₅₀ |
|---|---|---|
| NUMBER | NAME | μM |
| 1. | 2-(3-(3,4-difluorobenzyl)-4-oxo-3,4-dihydroquinazolin-6-ylamino)-2- | 2.1 |
| | oxoethyl acetate | |
| 2. | N-(3-(3,4-difluorobenzyl)-4-oxo-3,4-dihydroquinazolin-6- | 2.4 |
| | yl)-2-hydroxyacetamide | |
| 3. | 2-(3-(4-chlorobenzyl)-4-oxo-3,4-dihydroquinazolin-6-ylamino)-2- | 0.94 |
| | oxoethyl acetate | |
| 4. | N-(3-(4-chlorobenzyl)-4-oxo-3,4-dihydroquinazolin-6-yl)-2- | 1.2 |
| | hydroxyacetamide | |
| 5. | N-(3-(4-chlorobenzyl)-4-oxo-3,4-dihydroquinazolin-6-yl)-2- | 8 |
| | phenoxyacetamide | |
| 6. | 2-(3-(4-chloro-3-(trifluoromethyl)benzyl)-4-oxo-3,4- | 0.13 |
| | dihydroquinazolin-6-ylamino)-2-oxoethyl acetate | |
| 7. | N-(3-(4-chloro-3-(trifluoromethyl)benzyl)-4-oxo-3,4- | 0.19 |
| | dihydroquinazolin-6-yl)-2-hydroxyacetamide | |
| 8. | benzyl 3-(3,4-dichlorobenzyl)-2-methyl-4-oxo-3,4- | 1.2 |
| | dihydroquinazolin-7-ylcarbamate | |
| 9. | benzyl 3-(1-(3,4-dichlorophenyl)ethyl)-4-oxo-3,4- | 0.29 |
| | dihydroquinazolin-7-ylcarbamate | |
| 10. | 2-(3-(1-(3,4-dichlorophenyl)ethyl)-4-oxo-3,4- | 0.13 |
| | dihydroquinazolin-7-ylamino)-2-oxoethyl acetate | |
| 11. | N-(3-(1-(3,4-dichlorophenyl)ethyl)-4-oxo-3,4- | 0.17 |
| | dihydroquinazolin-7-yl)-2-hydroxyacetamide | |
| 12. | benzyl 3-(3-methoxybenzyl)-4-oxo-3,4-dihydroquinazolin-6- | 1.4 |
| | ylcarbamate | |
| 13. | 2-(3-(3-chloro-4-fluorobenzyl)-4-oxo-3,4-dihydroquinazolin- | 1.7 |
| | 6-ylamino)-2-oxoethyl acetate | |
| 14. | benzyl 3-(4-fluoro-3-(trifluoromethyl)benzyl)-4-oxo-3,4- | 0.31 |
| | dihydroquinazolin-6-ylcarbamate | |
| 15. | 2-(3-(4-fluoro-3-(trifluoromethyl)benzyl)-4-oxo-3,4- | 0.7 |
| | dihydroquinazolin-6-ylamino)-2-oxoethyl acetate | |
| 16. | N-(3-(4-fluoro-3-(trifluoromethyl)benzyl)-4-oxo-3,4- | 0.92 |
| | dihydroquinazolin-6-yl)-2-hydroxyacetamide | |
| 17. | N-(3-(3,4-dichlorobenzyl)-4-oxo-3,4-dihydroquinazolin-6- | 3.9 |
| | yl)acetamide | |
| 18. | N-(3-(3,4-difluorobenzyl)-4-oxo-3,4-dihydroquinazolin-6- | 26 |
| | yl)acetamide | |
| 19. | N-(4-oxo-3-(4-(trifluoromethyl)benzyl)-3,4- | 28 |
| | dihydroquinazolin-6-yl)acetamide | |
| 20. | benzyl 3-(3,4-dimethylbenzyl)-4-oxo-3,4-dihydroquinazolin- | 0.23 |
| | 6-ylcarbamate | |
| 21. | 2-(3-(3,4-dimethylbenzyl)-4-oxo-3,4-dihydroquinazolin-6- | 0.47 |
| | ylamino)-2-oxoethyl acetate | |
| 22. | N-(3-(3,4-dimethylbenzyl)-4-oxo-3,4-dihydroquinazolin-6- | 0.28 |
| | yl)-2-hydroxyacetamide | |
| 23. | N-(3-(1-(3,4-dichlorophenyl)ethyl)-4-oxo-3,4- | 0.21 |
| | dihydroquinazolin-6-yl)-2-hydroxyacetamide | |
| 24. | 2-oxo-2-(4-oxo-3-(3-phenylpropyl)-3,4-dihydroquinazolin-6- | 1.3 |
| | ylamino)ethyl acetate | |
| 25. | 2-(3-(1-(3,4-dichlorophenyl)propyl)-4-oxo-3,4- | 0.5 |
| | dihydroquinazolin-6-ylamino)-2-oxoethyl acetate | |
| 26. | N-(3-(1-(3,4-dichlorophenyl)propyl)-4-oxo-3,4- | 3.3 |
| | dihydroquinazolin-6-yl)-2-hydroxyacetamide | |
| 27. | 2-hydroxy-N-(4-oxo-3-(3-phenylpropyl)-3,4- | 4.5 |
| | dihydroquinazolin-6-yl)acetamide | |
| 28. | 2-oxo-2-(4-oxo-3-(2-(trifluoromethyl)benzyl)-3,4- | 1.5 |
| | dihydroquinazolin-6-ylamino)ethyl acetate | |
| 29. | N-(3-(biphenyl-3-ylmethyl)-4-oxo-3,4-dihydroquinazolin-6- | 3.9 |
| | yl)-2-hydroxyacetamide | |
| 30. | benzyl 3-(1-(3,4-dichlorophenyl)propyl)-4-oxo-3,4- | 1.4 |
| | dihydroquinazolin-6-ylcarbamate | |
| 31. | 2-hydroxy-N-(3-(naphthalen-2-ylmethyl)-4-oxo-3,4- | 0.34 |
| | dihydroquinazolin-6-yl)acetamide | |
| 32. | benzyl 3-(naphthalen-2-ylmethyl)-4-oxo-3,4- | 0.72 |
| | dihydroquinazolin-6-ylcarbamate | |
| 33. | 2-hydroxy-N-(3-(naphthalen-2-ylmethyl)-4-oxo-3,4- | 2.8 |
| | dihydroquinazolin-7-yl)acetamide | |
| 34. | (R)—N-(3-(1-(4-chloro-3-(trifluoromethyl)phenyl)ethyl)-4- | 0.48 |
| | oxo-3,4-dihydroquinazolin-6-yl)-2-hydroxyacetamide | |
| 35. | (S)—N-(3-(1-(3,4-dichlorophenyl)ethyl)-4-oxo-3,4- | 1.8 |
| | dihydroquinazolin-7-yl)-2-hydroxyacetamide | |
| 36. | (R)—N-(3-(1-(3,4-dichlorophenyl)ethyl)-4-oxo-3,4- | 0.058 |
| | dihydroquinazolin-7-yl)-2-hydroxyacetamide | |
| 37. | N-(3-(4-chlorophenethyl)-4-oxo-3,4-dihydroquinazolin-6- | 0.02 |
| | yl)-2-hydroxyacetamide | |
| 38. | N-(3-(3-chlorophenethyl)-4-oxo-3,4-dihydroquinazolin-6- | 5.8 |
| | yl)-2-hydroxyacetamide | |
| 39. | N-(3-(2,4-dichlorophenethyl)-4-oxo-3,4-dihydroquinazolin- | 4.9 |
| | 6-yl)-2-hydroxyacetamide | |
| 40. | benzyl 4-oxo-3-(3-(trifluoromethyl)benzyl)-3,4- | 1.4 |
| | dihydroquinazolin-6-ylcarbamate | |
| 41. | benzyl 4-oxo-3-(3-(2-(trifluoromethyl)phenoxy)propyl)-3,4- | 0.069 |
| | dihydroquinazolin-6-ylcarbamate | |
| 42. | N-(3-(2-(2,5-dichlorophenoxy)ethyl)-4-oxo-3,4- | 0.29 |
| | dihydroquinazolin-6-yl)-2-hydroxyacetamide | |
| 43. | N-(3-(3-(2,5-dichlorophenoxy)propyl)-4-oxo-3,4- | 0.055 |
| | dihydroquinazolin-6-yl)-2-hydroxyacetamide | |
| 44. | benzyl 3-(3-(2,5-dichlorophenoxy)propyl)-4-oxo-3,4- | 0.055 |
| | dihydroquinazolin-6-ylcarbamate | |
| 45. | 2-hydroxy-N-(4-oxo-3-(3-(2- | 0.12 |
| | (trifluoromethyl)phenoxy)propyl)-3,4-dihydroquinazolin-6- | |
| | yl)acetamide | |
| 46. | benzyl 3-(3-(2-fluorophenoxy)propyl)-4-oxo-3,4- | 0.49 |
| | dihydroquinazolin-6-ylcarbamate | |
| 47. | 2-(3-(3-(2-fluorophenoxy)propyl)-4-oxo-3,4- | 9.8 |
| | dihydroquinazolin-6-ylamino)-2-oxoethyl acetate | |
| 48. | N-(3-(3-(2-fluorophenoxy)propyl)-4-oxo-3,4- | 11 |
| | dihydroquinazolin-7-yl)-2-hydroxyacetamide | |
| 49. | benzyl 3-(3-(2-cyanophenoxy)propyl)-4-oxo-3,4- | 0.48 |
| | dihydroquinazolin-6-ylcarbamate | |
| 50. | benzyl 3-(3-(2,5-dichlorophenoxy)propyl)-4-oxo-3,4- | 0.45 |
| | dihydroquinazolin-7-ylcarbamate | |
| 51. | N-(3-(3-(2,5-dichlorophenoxy)propyl)-4-oxo-3,4- | 0.086 |
| | dihydroquinazolin-7-yl)-2-hydroxyacetamide | |
| 52. | benzyl 4-oxo-3-(3-(o-tolyloxy)propyl)-3,4- | 0.044 |
| | dihydroquinazolin-6-ylcarbamate | |
| 53. | 2-hydroxy-N-(4-oxo-3-(3-(o-tolyloxy)propyl)-3,4- | 0.1 |
| | dihydroquinazolin-6-yl)acetamide | |
| 54. | benzyl 3-(4-(2,5-dichlorophenoxy)butyl)-4-oxo-3,4- | 0.15 |
| | dihydroquinazolin-6-ylcarbamate | |
| 55. | N-(3-(4-(2,5-dichlorophenoxy)butyl)-4-oxo-3,4- | 0.24 |
| | dihydroquinazolin-6-yl)-2-hydroxyacetamide | |
| 56. | N-(3-(3-(2-chlorophenyl)propyl)-4-oxo-3,4- | 0.43 |
| | dihydroquinazolin-6-yl)-2-hydroxyacetamide | |
| 57. | benzyl 3-(2-(2-chlorophenoxy)ethyl)-4-oxo-3,4- | 0.18 |
| | dihydroquinazolin-6-ylcarbamate | |
| 58. | N-(3-(2-(2-chlorophenoxy)ethyl)-4-oxo-3,4- | 0.34 |
| | dihydroquinazolin-6-yl)-2-hydroxyacetamide | |
| 59. | benzyl 3-(3-(2-chlorophenoxy)propyl)-4-oxo-3,4- | 0.045 |
| | dihydroquinazolin-6-ylcarbamate | |
| 60. | N-(3-(3-(2-chlorophenoxy)propyl)-4-oxo-3,4- | 0.45 |
| | dihydroquinazolin-6-yl)-2-hydroxyacetamide | |
| 61. | N-(3-(3-((2,5-dichlorophenoxy)methyl)benzyl)-4-oxo-3,4- | 0.49 |
| | dihydroquinazolin-6-yl)-2-hydroxyacetamide | |
| 62. | benzyl 3-(3-((2,5-dichlorophenoxy)methyl)benzyl)-4-oxo- | 0.62 |
| | 3,4-dihydroquinazolin-6-ylcarbamate | |
| 63. | N-(3-(2-(2-chlorophenoxy)ethyl)-4-oxo-3,4- | 4.9 |
| | dihydroquinazolin-7-yl)-2-hydroxyacetamide | |
| 64. | N-(3-(5-(2,5-dichlorophenoxy)pentyl)-4-oxo-3,4- | 1.3 |
| | dihydroquinazolin-6-yl)-2-hydroxyacetamide | |
| 65. | N-(3-(3-(2,3-dichlorophenoxy)propyl)-4-oxo-3,4- | 0.64 |
| | dihydroquinazolin-6-yl)-2-hydroxyacetamide | |
| 66. | N-(3-(3-(2-chloro-5-(trifluoromethyl)phenoxy)propyl)-4- | 11 |
| | oxo-3,4-dihydroquinazolin-6-yl)-2-hydroxyacetamide | |
| 67. | 2,5-dichloro-N-(2-(6-(2-hydroxyacetamido)-4- | 21 |
| | oxoquinazolin-3(4H)-yl)ethyl)benzamide | |
| 68. | 2-hydroxy-N-(4-oxo-3-(3-phenoxypropyl)-3,4- | 23 |
| | dihydroquinazolin-6-yl)acetamide | |
| 69. | N-(2-(6-(2-hydroxyacetamido)-4-oxoquinazolin-3(4H)- | 5.2 |
| | yl)ethyl)-2-(trifluoromethyl)benzamide | |
| 70. | N-(3-(3-(4-chlorophenoxy)propyl)-4-oxo-3,4- | 31 |
| | dihydroquinazolin-6-yl)-2-hydroxyacetamide | |
| 71. | N-(3-(2-(2,5-dichlorophenylsulfonamido)ethyl)-4-oxo-3,4- | 15 |
| | dihydroquinazolin-6-yl)-2-hydroxyacetamide | |
| 72. | N-(3-(3-(2,5-dichlorophenylsulfonamido)propyl)-4-oxo-3,4- | 21 |
| | dihydroquinazolin-6-yl)-2-hydroxyacetamide | |
| 73. | 2-hydroxy-N-(4-oxo-3-(3-(2- | 14 |
| | (trifluoromethyl)phenylsulfonamido)propyl)-3,4- | |
| | dihydroquinazolin-6-yl)acetamide | |
| 74. | N-(3-(2-(4-chlorophenoxy)ethyl)-4-oxo-3,4- | 17 |
| | dihydroquinazolin-6-yl)-2-hydroxyacetamide | |
| 75. | N-(3-(2-(3-chlorophenoxy)ethyl)-4-oxo-3,4- | 1.5 |
| | dihydroquinazolin-6-yl)-2-hydroxyacetamide | |
| 76. | 2-hydroxy-N-(3-(2-hydroxy-3-(o-tolyloxy)propyl)-4-oxo- | 4 |
| | 3,4-dihydroquinazolin-6-yl)acetamide | |
| 77. | benzyl 3-(3,4-dichlorobenzyl)-4-oxo-3,4-dihydropyrido[2,3- | 0.36 |
| | d]pyrimidin-6-ylcarbamate | |
| 78. | N-(3-(3,4-dichlorobenzyl)-4-oxo-3,4-dihydropyrido[2,3- | 0.18 |
| | d]pyrimidin-6-yl)-2-hydroxyacetamide | |
| 79. | N-(3-(4-chlorobenzyl)-4-oxo-3,4-dihydropyrido[2,3- | 1.7 |
| | d]pyrimidin-6-yl)-2-hydroxyacetamide | |
| 80. | benzyl 3-(4-chlorobenzyl)-4-oxo-3,4-dihydropyrido[2,3- | 0.078 |
| | d]pyrimidin-6-ylcarbamate | |

We claim:

1. A compound having the structure of Formula I: 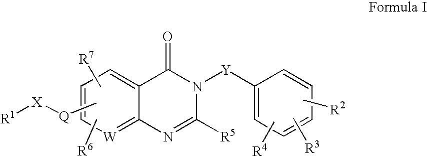
wherein
R¹ is hydrogen, C₁₋₁₅ alkyl, C₂₋₁₅ alkenyl, C₂₋₁₅ alkynyl, mono or bicyclic heterocyclyl, mono or bicyclic aryl, or mono or bicyclic heteroaryl,
wherein the alkyl, alkenyl, alkynyl, aryl, heterocyclyl or heteroaryl moiety is optionally substituted with from 1 to 3 substituents independently selected from the group consisting of alkyl, heterocyclyl, aryl, heteroaryl, halo, NO₂, CF₃, CN, OR²⁰, SR²⁰, N(R²⁰)₂, S(O)R²², SO₂R²², SO₂N(R²⁰)₂, S(O)₃R²⁰, P(O)(OR²⁰)₂, SO₂NR²⁰COR²², SO₂NR²⁰CO₂R²², SO₂NR²⁰CON(R²⁰)₂, NR²⁰COR²², NR²⁰CO₂R²², NR²⁰CON(R²⁰)₂, NR²⁰C(NR²⁰)NHR²³, COR²⁰, CO₂R²⁰, CON(R²⁰)₂, CONR²⁰SO₂R²², NR²⁰SO₂R²², SO₂NR²⁰CO₂R²², OCONR²⁰SO₂R²², OC(O)R²⁰, C(O)OCH₂OC(O)R²⁰, and OCON(R²⁰)₂, and
further wherein each optional alkyl, heteroaryl, aryl, and heterocyclyl substituent is further optionally substituted with halo, NO₂, alkyl, CF₃, amino, mono- or di-alkylamino, alkyl or aryl or heteroaryl amide, NR²⁰COR²², NR²⁰SO₂R²², COR²⁰, CO₂R²⁰, CON(R²⁰)₂, NR²⁰CON(R²⁰)₂, OC(O)R²⁰, OC(O)N(R²⁰)₂, S(O)₃R²⁰, P(O)(OR²⁰)₂, SR²⁰, S(O)R²², SO₂R²², SO₂N(R²⁰)₂, CN, or OR²⁰;
R², R³, and R⁴ are independently hydrogen, C₁₋₆ alkyl, C₂₋₆ alkenyl, C₂₋₆ alkynyl, mono or bicyclic heterocyclyl, mono or bicyclic aryl, mono or bicyclic heteroaryl, hydroxy, halo, NO₂, CF₃, CN, OR²⁰, SR²⁰, N(R²⁰)₂, S(O)R²², SO₂R²², SO₂N(R²⁰)₂, S(O)₃R²⁰, P(O)(OR²⁰)₂, SO₂NR²⁰COR²², SO₂NR²⁰CO₂R²², SO₂NR²⁰CON(R²⁰)₂, NR²⁰COR²², NR²⁰CO₂R²², NR²⁰CON(R²⁰)₂, NR²⁰C(NR²⁰)NHR²³, COR²⁰, CO₂R²⁰, CON(R²⁰)₂, CONR²⁰SO₂R²², NR²⁰SO₂R²², SO₂NR²⁰CO₂R²², OCONR²⁰SO₂R²², OC(O)R²⁰, C(O)OCH₂OC(O)R²⁰, OCON(R²⁰)₂,
wherein the alkyl, alkenyl, alkynyl, aryl, heterocyclyl, or heteroaryl moieties are optionally substituted with from 1 to 3 substituents independently selected from the group consisting of halo, alkyl, NO₂, heterocyclyl, aryl, heteroaryl, CF₃, CN, OR²⁰, SR²⁰, N(R²⁰)₂, S(O)R²², SO₂R²², SO₂N(R²⁰)₂, S(O)₃R²⁰, P(O)(OR²⁰)₂, SO₂NR²⁰COR²², SO₂NR²⁰CO₂R²², SO₂NR²⁰CON(R²⁰)₂, NR²⁰COR²², NR²⁰CO₂R²², NR²⁰CON(R²⁰)₂, NR²⁰C(NR²⁰)NHR²³, COR²⁰, CO₂R²⁰, CON(R²⁰)₂, CONR²⁰SO₂R²², NR²⁰SO₂R²², SO₂NR²⁰CO₂R²², OCONR²⁰SO₂R²², OC(O)R²⁰, C(O)OCH₂OC(O)R²⁰, and OCON(R²⁰); or
R² and R³ may join together with the phenyl group to which they are attached to form a heteroaryl bicyclic group or a bicyclic aryl group;
R⁵ is hydrogen, substituted alkyl, N(R²⁰)₂, NR²⁰COR²², NR²⁰CO₂R²², or NR²⁰CON(R²⁰)₂;
R⁶ and R⁷ are independently hydrogen or C₁₋₄ alkyl, halo, amino, or CF₃;
R⁸ is hydrogen, C₁₋₄ alkyl, alkenyl, alkynyl, aryl, heterocyclyl, heteroaryl, COR²⁰, or CON(R²⁰)₂;
wherein the alkyl, alkenyl, alkynyl, aryl, heterocyclyl and heteroaryl moiety is optionally substituted with from 1 to 3 substituents independently selected from the group consisting of halo, alkyl, NO₂, heterocyclyl, aryl, heteroaryl, CF₃, CN, OR²⁰, SR²⁰, N(R²⁰)₂, S(O)R²⁰, SO₂R²⁰, SO₂N(R²⁰)₂, COR²⁰, CO₂R²⁰, CON(R²⁰)₂, CONR²⁰SO₂R²², or NR²⁰SO₂R²²;
Q is —C(O)—NH—, —NH—C(O)—, or —NH—C(O)—C(O)—;
W is —CH— or —N—;
X is a covalent bond or -Lk-Lh-, wherein Lk is a covalent bond or optionally substituted linear or branched C₁₋₄ alkylene and Lh is selected from a covalent bond, —O—, —S—, or —NR″— wherein R″ is hydrogen or C₁₋₆ lower alkyl, provided that Lk and Lh are not both covalent bond;
Y is a covalent bond or -Lk′-Lh′-, wherein Lk′ is a covalent bond or optionally substituted linear or branched C₁₋₆ alkylene and Lh′ is selected from a covalent bond, —O—, —S—, —NR″—, —NR″—C(O)—, or —NR″—S(O)₂— wherein R″ is hydrogen or C₁₋₆ lower alkyl, provided that Lk′ and Lh′ are not both covalent bond; and
R²⁰ and R²² are independently selected from the group consisting of hydrogen, C₁₋₁₅ alkyl, C₂₋₁₅ alkenyl, C₂₋₁₅ alkynyl, heterocyclyl, aryl, and heteroaryl,
wherein the alkyl, alkenyl, alkynyl, heterocyclyl, aryl, and heteroaryl moieties are optionally substituted with from 1 to 3 substituents independently selected from halo, alkyl, mono- or dialkylamino, alkyl or aryl or heteroaryl amide, CN, O—C₁₋₆ alkyl, CF₃, aryl, and heteroaryl.

2. The compound of claim 1, wherein R¹ is hydrogen, mono or bicyclic aryl, mono or bicyclic heteroaryl, C₁₋₈ alkyl, or C₂₋₈ alkenyl,
wherein the aryl, heteroaryl, alky, or alkynyl moiety may be optionally substituted with halo, hydroxy, oxo, amino, acyloxy, acylamino, alkoxy, mono or bicyclic cycloalkyl, mono or bicyclic heterocyclyl, mono or bicyclic aryl, or mono or bicyclic heteroaryl, and
further wherein the amino, acyloxy, acylamino, alkoxy, cycloalkyl, heterocyclyl, aryl, or heteroaryl substituent may be further substituted with halo, hydroxy, oxo, nitro, cyano, amino, C₁₋₃ alkoxy, or C₁₋₅ alkyl optionally substituted with halo, hydroxy, methoxy, oxo, nitro, cyano, phenyl, or amino;
R², R³, and R⁴ are hydrogen, hydroxy, halo, C₁₋₅ alkyl or alkoxy optionally substituted with halo; or
R² and R³ may join along with the phenyl group to which they are attached to form a heteroaryl bicyclic group; and
R⁵ is hydrogen or methyl.

3. The compound of claim 1, wherein the compound has the structure of Formula Ia:

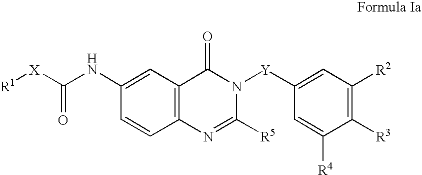

4. The compound of claim 1, wherein the compound has the structure of Formula Ib:

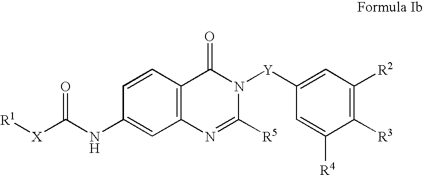

5. The compound of claim 1 wherein, Y is methylene.

6. The compound of claim 5, wherein X is a covalent bond.

7. The compound of claim 6, wherein R¹ is C₁₋₈ alkyl.

8. The compound of claim 7, namely N-{3-[(3,4-dichlorophenyl)methyl]-4-oxo-3-hydroquinazolin-6-yl}hexanamide.

9. The compound of claim 6, wherein R¹ is C₁₋₈ alkyl substituted with aryl.

10. The compound of claim 9, selected from the group consisting of:
N-{3-[(3,4-dichlorophenyl)methyl]-4-oxo(3-hydroquinazolin-6-yl)}-3-phenylpropanamide;
N-{3-[(3,4-dichlorophenyl)methyl]-4-oxo(3-hydroquinazolin-6-yl)}-4-oxo-4-phenylbutanamide; and
N-(3-(3,4-difluorobenzyl)-4-oxo-3,4-dihydroquinazolin-6-yl)-2-hydroxyacetamide.

11. The compound of claim 6, wherein R¹ is C₁₋₈ alkyl substituted with amino.

12. The compound of claim 11, namely 2-(acetylamino)-N-{3-[(3,4-dichlorophenyl)methyl]-4-oxo(3-hydroquinazolin-6-yl)}acetamide.

13. The compound of claim 6, wherein R¹ is C₁₋₈ alkyl substituted with hydroxy, carboxy, or acyloxy.

14. The compound of claim 13, selected from the group consisting of:
(N-{3-[(3,4-dichlorophenyl)methyl]-4-oxo-3-hydroquinazolin-6-yl}carbamoyl)methyl acetate;
N-{3-[(3,4-dichlorophenyl)methyl]-4-oxo(3-hydroquinazolin-6-yl)}-2-hydroxyacetamide;
(N-{3-[(3,4-dichlorophenyl)methyl]-4-oxo-3-hydroquinazolin-7-yl}carbamoyl)methyl acetate;
(N-{3-[(3-chlorophenyl)methyl]-4-oxo-3-hydroquinazolin-7-yl}carbamoyl)methyl acetate;
(N-{3-[(4-chlorophenyl)methyl]-4-oxo-3-hydroquinazolin-7-yl}carbamoyl)methyl acetate;
N-{3-[(3-chlorophenyl)methyl]-4-oxo(3-hydroquinazolin-7-yl)}-2-hydroxyacetamide;
(N-{3-[(3-fluorophenyl)methyl]-4-oxo-3-hydroquinazolin-7-yl}carbamoyl)methyl acetate;
(N-{3-[(3-chloro-4-fluorophenyl)methyl]-4-oxo-3-hydroquinazolin-7-yl}carbamoyl)methyl acetate;
(N-{3-[(4-chloro-3-fluorophenyl)methyl]-4-oxo-3-hydroquinazolin-7-yl}carbamoyl)methyl acetate;
N-{3-[(4-chlorophenyl)methyl]-4-oxo(3-hydroquinazolin-7-yl)}-2-hydroxyacetamide;
N-{3-[(3,4-dichlorophenyl)methyl]-4-oxo(3-hydroquinazolin-7-yl)}-2-hydroxyacetamide;
(N-{3-[(3,4-dichlorophenyl)methyl]-4-oxo-3-hydroquinazolin-6-yl}carbamoyl)methyl 2-(dimethylamino)acetate;
N-{3-[(3-chloro-4-fluorophenyl)methyl]-4-oxo(3-hydroquinazolin-7-yl)}-2-hydroxyacetamide;
(N-{3-[(3,4-dichlorophenyl)methyl]-4-oxo-3-hydroquinazolin-7-yl}carbamoyl)methyl 2-(dimethylamino)acetate;
[N-(4-oxo-3-{[3-(trifluoromethyl)phenyl]methyl}-3-hydroquinazolin-6-yl)carbamoyl]methyl acetate;
2-hydroxy-N-(4-oxo-3-{[3-(trifluoromethyl)phenyl]methyl}(3-hydroquinazolin-6-yl))acetamide;
[N-(4-oxo-3-{[4-(trifluoromethyl)phenyl]methyl}-3-hydroquinazolin-6-yl)carbamoyl]methyl acetate;
2-hydroxy-N-(4-oxo-3-{[4-(trifluoromethyl)phenyl]methyl}(3-hydroquinazolin-6-yl))acetamide;
(N-{3-[(4-chloro-3-fluorophenyl)methyl]-4-oxo-3-hydroquinazolin-6-yl}carbamoyl)methyl acetate;
N-{3-[(4-chloro-3-fluorophenyl)methyl]-4-oxo(3-hydroquinazolin-6-yl)}-2-hydroxyacetamide;
(N-{3-[(4-methoxyphenyl)methyl]-4-oxo-3-hydroquinazolin-6-yl}carbamoyl)methyl acetate;
2-hydroxy-N-{3-[(4-methoxyphenyl)methyl]-4-oxo(3-hydroquinazolin-6-yl)}acetamide;
(N-{3-[(3,4-difluorophenyl)methyl]-4-oxo-3-hydroquinazolin-7-yl}carbamoyl)methyl acetate;
N-{3-[(3,4-difluorophenyl)methyl]-4-oxo(3-hydroquinazolin-7-yl)}-2-hydroxyacetamide;
2-(3-(3,4-difluorobenzyl)-4-oxo-3,4-dihydroquinazolin-6-ylamino)-2-oxoethyl acetate;
2-(3-(4-chlorobenzyl)-4-oxo-3,4-dihydroquinazolin-6-ylamino)-2-oxoethyl acetate;
N-(3-(4-chlorobenzyl)-4-oxo-3,4-dihydroquinazolin-6-yl)-2-hydroxyacetamide;
2-(3-(4-chloro-3-(trifluoromethyl)benzyl)-4-oxo-3,4-dihydroquinazolin-6-ylamino)-2-oxoethyl acetate;
N-(3-(4-chloro-3-(trifluoromethyl)benzyl)-4-oxo-3,4-dihydroquinazolin-6-yl)-2-hydroxyacetamide;
2-(3-(3-chloro-4-fluorobenzyl)-4-oxo-3,4-dihydroquinazolin-6-ylamino)-2-oxoethyl acetate;
2-(3-(4-fluoro-3-(trifluoromethyl)benzyl)-4-oxo-3,4-dihydroquinazolin-6-ylamino)-2-oxoethyl acetate;
N-(3-(4-fluoro-3-(trifluoromethyl)benzyl)-4-oxo-3,4-dihydroquinazolin-6-yl)-2-hydroxyacetamide;
2-(3-(3,4-dimethylbenzyl)-4-oxo-3,4-dihydroquinazolin-6-ylamino)-2-oxoethyl acetate;
N-(3-(3,4-dimethylbenzyl)-4-oxo-3,4-dihydroquinazolin-6-yl)-2-hydroxyacetamide;
N-(3-benzyl-4-oxo-3,4-dihydroquinazolin-6-yl)-2-hydroxyacetamide;
2-oxo-2-(4-oxo-3-(2-(trifluoromethyl)benzyl)-3,4-dihydroquinazolin-6-ylamino)ethyl acetate;
N-(3-(biphenyl-3-ylmethyl)-4-oxo-3,4-dihydroquinazolin-6-yl)-2-hydroxyacetamide;
2-hydroxy-N-(3-(naphthalen-2-ylmethyl)-4-oxo-3,4-dihydroquinazolin-6-yl)acetamide; and
2-hydroxy-N-(3-(naphthalen-2-ylmethyl)-4-oxo-3,4-dihydroquinazolin-7-yl)acetamide.

15. The compound of claim 6, wherein R¹ is C₁₋₈ alkyl substituted with aryloxy.

16. The compound of claim 15, selected from the group consisting of:
N-{3-[(3,4-dichlorophenyl)methyl]-4-oxo(3-hydroquinazolin-6-yl)}-2-phenoxyacetamide;
N-{3-[(3,4-dichlorophenyl)methyl]-4-oxo(3-hydroquinazolin-6-yl)}-2-(4-methoxyphenoxy)acetamide;
N-{3-[(3,4-dichlorophenyl)methyl]-4-oxo(3-hydroquinazolin-6-yl)}-2-(4-fluorophenoxy)acetamide;
N-{3-[(3,4-dichlorophenyl)methyl]-4-oxo(3-hydroquinazolin-6-yl)}-2-(3,5-dimethylphenoxy)acetamide;
N-{3-[(3,4-dichlorophenyl)methyl]-4-oxo(3-hydroquinazolin-6-yl)}-2-(2,6-dimethylphenoxy)acetamide;
N-{3-[(3,4-dichlorophenyl)methyl]-4-oxo(3-hydroquinazolin-6-yl)}-2-(4-methylphenoxy)acetamide;
N-{3-[(3,4-dichlorophenyl)methyl]-4-oxo(3-hydroquinazolin-6-yl)}-2-(2-methoxyphenoxy)acetamide;
N-{3-[(3,4-dichlorophenyl)methyl]-4-oxo(3-hydroquinazolin-6-yl)}-2-(2-fluorophenoxy)acetamide;
N-{3-[(3,4-dichlorophenyl)methyl]-4-oxo(3-hydroquinazolin-6-yl)}-2-(3-fluorophenoxy)acetamide;
N-{3-[(3,4-dichlorophenyl)methyl]-4-oxo(3-hydroquinazolin-6-yl)}-2-(2,4-difluorophenoxy)acetamide;
N-{3-[(3,4-dichlorophenyl)methyl]-4-oxo(3-hydroquinazolin-6-yl)}-2-(2-chlorophenoxy)acetamide;
N-{3-[(3,4-dichlorophenyl)methyl]-4-oxo(3-hydroquinazolin-6-yl)}-2-(2,3-dichlorophenoxy)acetamide;
N-{3-[(3,4-dichlorophenyl)methyl]-4-oxo(3-hydroquinazolin-6-yl)}-2-(2-nitrophenoxy)acetamide;
N-{3-[(3,4-dichlorophenyl)methyl]-4-oxo(3-hydroquinazolin-6-yl)}-2-(2-naphthyloxy)acetamide;
N-{3-[(3,4-dichlorophenyl)methyl]-4-oxo(3-hydroquinazolin-6-yl)}-3-phenoxypropanamide;
N-{3-[(3,4-dichlorophenyl)methyl]-4-oxo(3-hydroquinazolin-6-yl)}-2-(2-chlorophenoxy)propanamide;
N-{3-[(4-bromophenyl)methyl]-4-oxo(3-hydroquinazolin-6-yl)}-2-phenoxyacetamide;
N-{3-[(4-chloro-3-fluorophenyl)methyl]-4-oxo(3-hydroquinazolin-6-yl)}-2-phenoxyacetamide;
N-{3-[(3-chloro-4-fluorophenyl)methyl]-4-oxo(3-hydroquinazolin-6-yl)}-2-phenoxyacetamide;
N-[3-(benzo[b]thiophen-6-ylmethyl)-4-oxo(3-hydroquinazolin-6-yl)]-2-phenoxyacetamide;
N-{3-[(4-chlorophenyl)methyl]-4-oxo(3-hydroquinazolin-6-yl)}-2-(3-fluorophenoxy)acetamide;
N-{3-[(4-chlorophenyl)methyl]-4-oxo(3-hydroquinazolin-7-yl)}-2-phenoxyacetamide;
N-{3-[(3,4-dichlorophenyl)methyl]-4-oxo(3-hydroquinazolin-7-yl)}-2-(2-methoxyphenoxy)acetamide;
N-{3-[(3,4-dichlorophenyl)methyl]-4-oxo(3-hydroquinazolin-7-yl)}-2-phenoxyacetamide; and
N-(3-(4-chlorobenzyl)-4-oxo-3,4-dihydroquinazolin-6-yl)-2-phenoxyacetamide.

17. The compound of claim 6, wherein R¹ is C₁₋₈ alkyl substituted with heteroaryloxy.

18. The compound of claim 17, selected from the group consisting of:
2-benzo[c]1,2,5-thiadiazol-4-yloxy-N-{3-[(3,4-dichlorophenyl)methyl]-4-oxo(3-hydroquinazolin-6-yl)}acetamide;
N-{3-[(3,4-dichlorophenyl)methyl]-4-oxo(3-hydroquinazolin-6-yl)}-2-(3-pyridyloxy)acetamide;
N-{3-[(4-chlorophenyl)methyl]-4-oxo(3-hydroquinazolin-7-yl)}-2-(3-pyridyloxy)acetamide; and
N-{3-[(3,4-dichlorophenyl)methyl]-4-oxo(3-hydroquinazolin-7-yl)}-2-(3-pyridyloxy)acetamide.

19. The compound of claim 6, wherein R¹ is C₁₋₈ alkyl substituted with arylthioalkyl, heteroarylthioalkyl, or arylalkylthioalkyl.

20. The compound of claim 19, selected from the group consisting of:
N-{3-[(3,4-dichlorophenyl)methyl]-4-oxo(3-hydroquinazolin-6-yl)}-2-(4-chlorophenylthio)acetamide;
N-{3-[(4-chlorophenyl)methyl]-4-oxo(3-hydroquinazolin-6-yl)}-2-(4-pyridylthio)acetamide;
N-{3-[(3,4-dichlorophenyl)methyl]-4-oxo(3-hydroquinazolin-6-yl)}-2-(4-pyridylthio)acetamide;
N-{3-[(3,4-dichlorophenyl)methyl]-4-oxo(3-hydroquinazolin-6-yl)}-2-pyrimidin-2-ylthioacetamide; and
N-{3-[(3,4-dichlorophenyl)methyl]-4-oxo(3-hydroquinazolin-6-yl)}-2-(phenylmethylthio)acetamide.

21. The compound of claim 5, wherein X is NH.

22. The compound of claim 21, selected from the group consisting of:
N-{3-[(3,4-dichlorophenyl)methyl]-4-oxo(3-hydroquinazolin-6-yl)}(pentylamino)carboxamide;
N-{3-[(3,4-dichlorophenyl)methyl]-4-oxo(3-hydroquinazolin-6-yl)}[benzylamino]carboxamide; and
N-{3-[(3,4-dichlorophenyl)methyl]-4-oxo(3-hydroquinazolin-6-yl)}[(2-methoxyethyl)amino]carboxamide.

23. The compound of claim 5, wherein X is O.

24. The compound of claim 23, wherein R¹ is C₁₋₈ alkyl or C₂₋₈ alkenyl optionally substituted with optionally substituted alkoxy, optionally substituted amino, cycloalkyl, or carboxy.

25. The compound of claim 24, selected from the group consisting of:
N-{3-[(3,4-dichlorophenyl)methyl]-4-oxo(3-hydroquinazolin-6-yl)}(2-methylpropoxy)carboxamide;
N-{3-[(3,4-dichlorophenyl)methyl]-4-oxo(3-hydroquinazolin-6-yl)}(methylethoxy)carboxamide;
N-{3-[(3,4-dichlorophenyl)methyl]-4-oxo(3-hydroquinazolin-6-yl)}prop-2-enyloxycarboxamide;
N-{3-[(3,4-dichlorophenyl)methyl]-4-oxo(3-hydroquinazolin-6-yl)}methoxycarboxamide;
N-{3-[(4-chlorophenyl)methyl]-4-oxo(3-hydroquinazolin-6-yl)}prop-2-enyloxycarboxamide;
N-{3-[(3,4-dichlorophenyl)methyl]-4-oxo(3-hydroquinazolin-7-yl)}(2-methoxyethoxy)carboxamide;
N-{3-[(3,4-dichlorophenyl)methyl]-4-oxo(3-hydroquinazolin-6-yl)}{2-[methylbenzylamino]ethoxy}carboxamide;
N-{3-[(3,4-dichlorophenyl)methyl]-4-oxo(3-hydroquinazolin-6-yl)}(cyclopropylmethoxy)carboxamide;
ethyl 2-(N-{3-[(3,4-dichlorophenyl)methyl]-4-oxo-3-hydroquinazolin-6-yl}carbamoyloxy)acetate;
ethyl 2-(N-{3-[(3,4-dichlorophenyl)methyl]-4-oxo-3-hydroquinazolin-7-yl}carbamoyloxy)acetate; and
N-{3-[(3,4-dichlorophenyl)methyl]-4-oxo(3-hydroquinazolin-6-yl)}[2-(2-methoxyethoxy)ethoxy]carboxamide.

26. The compound of claim 23, wherein R¹ is C₁₋₈ alkyl substituted with aryl.

27. The compound of claim 26, selected from the group consisting of:
N-{3-[(3,4-dichlorophenyl)methyl]-4-oxo(3-hydroquinazolin-6-yl)}(phenylmethoxy)carboxamide;
N-{3-[(3,4-dichlorophenyl)methyl]-2-methyl-4-oxo(3-hydroquinazolin-6-yl)}(phenylmethoxy)carboxamide;
N-{3-[(4-chlorophenyl)methyl]-4-oxo(3-hydroquinazolin-6-yl)}(phenylmethoxy)carboxamide;
N-{3-[(4-fluorophenyl)methyl]-4-oxo(3-hydroquinazolin-6-yl)}(phenylmethoxy)carboxamide;
N-{3-[(3-chloro-4-fluorophenyl)methyl]-4-oxo(3-hydroquinazolin-6-yl)}(phenylmethoxy)carboxamide;
N-{3-[(4-chlorophenyl)methyl]-4-oxo(3-hydroquinazolin-7-yl)}(phenylmethoxy)carboxamide;
N-{3-[(3,4-dichlorophenyl)methyl]-4-oxo(3-hydroquinazolin-7-yl)}(phenylmethoxy)carboxamide;
N-{3-[(3-chlorophenyl)methyl]-4-oxo(3-hydroquinazolin-7-yl)}(phenylmethoxy)carboxamide;
N-{3-[(3,4-dichlorophenyl)methyl]-4-oxo(3-hydroquinazolin-7-yl)}[(2-methoxyphenyl)methoxy]carboxamide;
N-{3-[(3-fluorophenyl)methyl]-4-oxo(3-hydroquinazolin-7-yl)}(phenylmethoxy)carboxamide;
N-{3-[(3,4-dichlorophenyl)methyl]-4-oxo(3-hydroquinazolin-6-yl)}(2-phenylethoxy)carboxamide;
N-{3-[(3-chloro-4-fluorophenyl)methyl]-4-oxo(3-hydroquinazolin-7-yl)}(phenylmethoxy)carboxamide;
N-{3-[(3,4-dichlorophenyl)methyl]-4-oxo(3-hydroquinazolin-6-yl)}[(3-cyanophenyl)methoxy]carboxamide;
N-{3-[(4-chloro-3-fluorophenyl)methyl]-4-oxo(3-hydroquinazolin-7-yl)}(phenylmethoxy)carboxamide;
N-{3-[2-(4-chlorophenyl)ethyl]-4-oxo(3-hydroquinazolin-6-yl)}(phenylmethoxy)carboxamide;
N-[3-(benzo[b]thiophen-5-ylmethyl)-4-oxo(3-hydroquinazolin-6-yl)](phenylmethoxy)carboxamide;
benzyl 4-oxo-3-(3-(trifluoromethyl)benzyl)-3,4-dihydroquinazolin-6-ylcarbamate;
benzyl 3-(3,4-dichlorobenzyl)-2-methyl-4-oxo-3,4-dihydroquinazolin-7-ylcarbamate;
benzyl 3-(3-methoxybenzyl)-4-oxo-3,4-dihydroquinazolin-6-ylcarbamate;
benzyl 3-(4-fluoro-3-(trifluoromethyl)benzyl)-4-oxo-3,4-dihydroquinazolin-6-ylcarbamate;
benzyl 3-(3,4-dimethylbenzyl)-4-oxo-3,4-dihydroquinazolin-6-ylcarbamate;
benzyl 3-(naphthalen-2-ylmethyl)-4-oxo-3,4-dihydroquinazolin-6-ylcarbamate;
benzyl 3-(3-((2,5-dichlorophenoxy)methyl)benzyl)-4-oxo-3,4-dihydroquinazolin-6-ylcarbamate.

28. The compound of claim 23, wherein R¹ is optionally substituted aryl.

29. The compound of claim 28, selected from the group consisting of:
N-{3-[(3,4-dichlorophenyl)methyl]-4-oxo(3-hydroquinazolin-6-yl)}(4-methylphenoxy)carboxamide;
N-{3-[(3,4-dichlorophenyl)methyl]-4-oxo(3-hydroquinazolin-6-yl)}(4-fluorophenoxy)carboxamide; and
N-{3-[(3,4-dichlorophenyl)methyl]-4-oxo(3-hydroquinazolin-6-yl)}(4-chlorophenoxy)carboxamide.

30. The compound of claim 23, wherein R¹ is C₁₋₈ alkyl substituted with heteroaryl.

31. The compound of claim 30, selected from the group consisting of:
N-{3-[(3,4-dichlorophenyl)methyl]-4-oxo(3-hydroquinazolin-6-yl)}(3-pyridylmethoxy)carboxamide;
N-{3-[(3-chlorophenyl)methyl]-4-oxo(3-hydroquinazolin-7-yl)}(3-pyridylmethoxy)carboxamide;
N-{3-[(3,4-dichlorophenyl)methyl]-4-oxo(3-hydroquinazolin-6-yl)}(2-furylmethoxy)carboxamide;
N-{3-[(3-chloro-4-fluorophenyl)methyl]-4-oxo(3-hydroquinazolin-7-yl)}(3-pyridylmethoxy)carboxamide;
N-{3-[(3-fluorophenyl)methyl]-4-oxo(3-hydroquinazolin-7-yl)}(3-pyridylmethoxy)carboxamide;
N-{3-[(4-chloro-3-fluorophenyl)methyl]-4-oxo(3-hydroquinazolin-7-yl)}(3-pyridylmethoxy)carboxamide;
N-{3-[(3,4-dichlorophenyl)methyl]-4-oxo(3-hydroquinazolin-6-yl)}(4-pyridylmethoxy)carboxamide; and
N-{3-[(3,4-dichlorophenyl)methyl]-4-oxo(3-hydroquinazolin-6-yl)}(2-pyridylmethoxy)carboxamide.

32. The compound of claim 1 wherein, Y is -Lk′-Lh′-, wherein Lk′ is optionally substituted linear or branched C₂₋₆ alkylene and Lh′ is selected from a covalent bond, —O—, —S—, —NR″—, —NR″—C(O)—, or —NR″—S(O)₂— wherein R″ is hydrogen or C₁₋₆ lower alkyl.

33. The compound of claim 32, selected from the group consisting of:
N-(3-(4-chlorophenethyl)-4-oxo-3,4-dihydroquinazolin-6-yl)-2-hydroxyacetamide;
N-(3-(3-chlorophenethyl)-4-oxo-3,4-dihydroquinazolin-6-yl)-2-hydroxyacetamide;
N-(3-(2,4-dichlorophenethyl)-4-oxo-3,4-dihydroquinazolin-6-yl)-2-hydroxyacetamide;
2,5-dichloro-N-(2-(6-(2-hydroxyacetamido)-4-oxoquinazolin-3(4H)-yl)ethyl)benzamide;
N-(2-(6-(2-hydroxyacetamido)-4-oxoquinazolin-3(4H)-yl)ethyl)-2-(trifluoromethyl)benzamide;
N-(3-(2-(2,5-dichlorophenylsulfonamido)ethyl)-4-oxo-3,4-dihydroquinazolin-6-yl)-2-hydroxyacetamide;
N-(3-(2-(2,5-dichlorophenoxy)ethyl)-4-oxo-3,4-dihydroquinazolin-6-yl)-2-hydroxyacetamide;
benzyl 3-(2-(2-chlorophenoxy)ethyl)-4-oxo-3,4-dihydroquinazolin-6-ylcarbamate;
N-(3-(2-(2-chlorophenoxy)ethyl)-4-oxo-3,4-dihydroquinazolin-6-yl)-2-hydroxyacetamide;
N-(3-(2-(2-chlorophenoxy)ethyl)-4-oxo-3,4-dihydroquinazolin-7-yl)-2-hydroxyacetamide;
N-(3-(2-(4-chlorophenoxy)ethyl)-4-oxo-3,4-dihydroquinazolin-6-yl)-2-hydroxyacetamide;
N-(3-(2-(3-chlorophenoxy)ethyl)-4-oxo-3,4-dihydroquinazolin-6-yl)-2-hydroxyacetamide;
N-(3-(3-(2-chlorophenyl)propyl)-4-oxo-3,4-dihydroquinazolin-6-yl)-2-hydroxyacetamide;
N-(3-(3-(2,5-dichlorophenylsulfonamido)propyl)-4-oxo-3,4-dihydroquinazolin-6-yl)-2-hydroxyacetamide;
2-hydroxy-N-(4-oxo-3-(3-(2-(trifluoromethyl)phenylsulfonamido)propyl)-3,4-dihydroquinazolin-6-yl)acetamide;
benzyl 4-oxo-3-(3-(2-(trifluoromethyl)phenoxy)propyl)-3,4-dihydroquinazolin-6-ylcarbamate;
N-(3-(3-(2,5-dichlorophenoxy)propyl)-4-oxo-3,4-dihydroquinazolin-6-yl)-2-hydroxyacetamide;
benzyl 3-(3-(2,5-dichlorophenoxy)propyl)-4-oxo-3,4-dihydroquinazolin-6-ylcarbamate;
2-hydroxy-N-(4-oxo-3-(3-(2-(trifluoromethyl)phenoxy)propyl)-3,4-dihydroquinazolin-6-yl)acetamide;
benzyl 3-(3-(2-fluorophenoxy)propyl)-4-oxo-3,4-dihydroquinazolin-6-ylcarbamate;
2-(3-(3-(2-fluorophenoxy)propyl)-4-oxo-3,4-dihydroquinazolin-6-ylamino)-2-oxoethyl acetate;
N-(3-(3-(2-fluorophenoxy)propyl)-4-oxo-3,4-dihydroquinazolin-7-yl)-2-hydroxyacetamide;
benzyl 3-(3-(2-cyanophenoxy)propyl)-4-oxo-3,4-dihydroquinazolin-6-ylcarbamate;
benzyl 3-(3-(2,5-dichlorophenoxy)propyl)-4-oxo-3,4-dihydroquinazolin-7-ylcarbamate;
N-(3-(3-(2,5-dichlorophenoxy)propyl)-4-oxo-3,4-dihydroquinazolin-7-yl)-2-hydroxyacetamide;
benzyl 4-oxo-3-(3-(o-tolyloxy)propyl)-3,4-dihydroquinazolin-6-ylcarbamate;
2-hydroxy-N-(4-oxo-3-(3-(o-tolyloxy)propyl)-3,4-dihydroquinazolin-6-yl)acetamide;
benzyl 3-(3-(2-chlorophenoxy)propyl)-4-oxo-3,4-dihydroquinazolin-6-ylcarbamate;
N-(3-(3-(2-chlorophenoxy)propyl)-4-oxo-3,4-dihydroquinazolin-6-yl)-2-hydroxyacetamide;
N-(3-(3-(2,3-dichlorophenoxy)propyl)-4-oxo-3,4-dihydroquinazolin-6-yl)-2-hydroxyacetamide;
N-(3-(3-(2-chloro-5-(trifluoromethyl)phenoxy)propyl)-4-oxo-3,4-dihydroquinazolin-6-yl)-2-hydroxyacetamide;
2-hydroxy-N-(4-oxo-3-(3-phenoxypropyl)-3,4-dihydroquinazolin-6-yl)acetamide;
N-(3-(3-(4-chlorophenoxy)propyl)-4-oxo-3,4-dihydroquinazolin-6-yl)-2-hydroxyacetamide;
benzyl 3-(4-(2,5-dichlorophenoxy)butyl)-4-oxo-3,4-dihydroquinazolin-6-ylcarbamate;
N-(3-(4-(2,5-dichlorophenoxy)butyl)-4-oxo-3,4-dihydroquinazolin-6-yl)-2-hydroxyacetamide;
N-(3-(5-(2,5-dichlorophenoxy)pentyl)-4-oxo-3,4-dihydroquinazolin-6-yl)-2-hydroxyacetamide;
(R)—N-(3-(1-(4-chloro-3-(trifluoromethyl)phenyl)ethyl)-4-oxo-3,4-dihydroquinazolin-6-yl)-2-hydroxyacetamide;
(S)—N-(3-(1-(3,4-dichlorophenyl)ethyl)-4-oxo-3,4-dihydroquinazolin-7-yl)-2-hydroxyacetamide;
(R)—N-(3-(1-(3,4-dichlorophenyl)ethyl)-4-oxo-3,4-dihydroquinazolin-7-yl)-2-hydroxyacetamide;
2-hydroxy-N-(3-(2-hydroxy-3-(o-tolyloxy)propyl)-4-oxo-3,4-dihydroquinazolin-6-yl)acetamide;
2-hydroxy-N-(4-oxo-3-phenethyl-3,4-dihydroquinazolin-6-yl)acetamide;
2-oxo-2-(4-oxo-3-(3-phenylpropyl)-3,4-dihydroquinazolin-6-ylamino)ethyl acetate;
2-hydroxy-N-(4-oxo-3-(3-phenylpropyl)-3,4-dihydroquinazolin-6-yl)acetamide;
benzyl 3-(1-(3,4-dichlorophenyl)ethyl)-4-oxo-3,4-dihydroquinazolin-7-ylcarbamate;
2-(3-(1-(3,4-dichlorophenyl)ethyl)-4-oxo-3,4-dihydroquinazolin-7-ylamino)-2-oxoethyl acetate;
N-(3-(1-(3,4-dichlorophenyl)ethyl)-4-oxo-3,4-dihydroquinazolin-7-yl)-2-hydroxyacetamide;
N-(3-(1-(3,4-dichlorophenyl)ethyl)-4-oxo-3,4-dihydroquinazolin-6-yl)-2-hydroxyacetamide;
2-(3-(1-(3,4-dichlorophenyl)propyl)-4-oxo-3,4-dihydroquinazolin-6-ylamino)-2-oxoethyl acetate;
N-(3-(1-(3,4-dichlorophenyl)propyl)-4-oxo-3,4-dihydroquinazolin-6-yl)-2-hydroxyacetamide; and
benzyl 3-(1-(3,4-dichlorophenyl)propyl)-4-oxo-3,4-dihydroquinazolin-6-ylcarbamate.

34. A pharmaceutical composition comprising a therapeutically effective amount of the compound of claim 1 or a pharmaceutically acceptable salt, ester, prodrug, or hydrate thereof.

35. A method for treating a disease or condition in a mammal that can be treated with an stearoyl-CoA desaturase inhibitory compound comprising administering to a mammal in need thereof a therapeutically effective dose of a compound of claim 1, or a pharmaceutically acceptable salt, ester, prodrug, solvate, or hydrate thereof.

36. The method of claim 35, wherein the disease state is selected from the group consisting of coronary artery disease, atherosclerosis, heart disease, hypertension, and peripheral vascular disease, cancer, cerebrovascular diseases (including, but not limited to, stroke, ischemic stroke and transient ischemic attack (TIA), and ischemic retinopathy), dyslipidemia, obesity, diabetes, insulin resistance, decreased glucose tolerance, non-insulin-dependent diabetes mellitus, Type II diabetes, Type I diabetes, and other diabetic complications.
